(19) United States
(12) Patent Application Publication
(10) Pub. No.: US 2025/0236667 A1
(43) Pub. Date: Jul. 24, 2025

(54) ANTIBODIES TO MUTANT CALRETICULIN AND USES THEREOF

(71) Applicants: CENTRAL ADELAIDE LOCAL HEALTH NETWORK INC, Adelaide (AU); UNIVERSITY OF SOUTH AUSTRALIA, Adelaide (AU); THE UNIVERSITY OF ADELAIDE, Adelaide (AU)

(72) Inventors: Denis TVOROGOV, Salisbury (AU); Angel LOPEZ, Medindie (AU); Daniel THOMAS, North Adelaide (AU)

(21) Appl. No.: 18/719,516

(22) PCT Filed: Dec. 12, 2022
(86) PCT No.: PCT/AU2022/051491

§ 371 Date: Jun. 13, 2024

(30) Foreign Application Priority Data
Dec. 13, 2021 (AU) 2021904034

(51) Int. Cl. C07K 16/28; A61K 40/31; A61K 40/41; A61P 35/00

(52) U.S. Cl. CPC C07K 16/28; A61K 40/31; A61K 40/41; A61P 35/00; A61K 2239/13; A61K 2239/48; C07K 2317/24; C07K 2317/565

(57) ABSTRACT

The present invention relates generally to methods for preventing and/or treating myeloproliferative disorders. More specifically, the myeloproliferative disorders are those associated with the presence of a frameshift mutation in the calreticulin (CALR) gene of a cell involved in the myeloproliferative disorders. The present invention also relates to immunological agents, such as antibodies, to calreticulin, methods for preventing and/or treating myeloproliferative diseases using the immunological agents, and chimeric antigen receptors having extracellular domains based on antibodies to calreticulin.

### PRIORITY CLAIM

This application is the U.S. National Stage of PCT/AU2022/051491, filed Dec. 12, 2022, which claims priority from Australian provisional patent application number 2021904034 filed on 13 Dec. 2021, the content of each of which is to be taken as incorporated herein by this reference.

### SEQUENCE LISTING

This application contains a sequence listing submitted electronically as a Standard ST.26 compliant XML file, with a file name of FF63276-24 Replacement Sequence Listing.xml,” a creation date of Aug. 6, 2024, and a file size of 40,960 bytes, the contents of which are incorporated herein.

### FIELD

The present disclosure relates to methods for preventing and/or treating myeloproliferative diseases, and also to antibodies and other immunological agents to calreticulin, methods for preventing and/or treating myeloproliferative diseases using the antibodies and immunological agents, and chimeric antigen receptors having extracellular domains based on the antibodies.

### BACKGROUND

Primary myelofibrosis is a rare bone marrow disorder that is characterised by abnormalities in blood cell production and scarring within the bone marrow. The disease belongs to a group of myeloproliferative diseases and includes essential thrombocythemia and secondary acute myeloid leukemia.

Primary myelofibrosis (PMF) is the most severe Philadelphia-negative myeloproliferative neoplasm, and is characterised by marrow fibrosis and chronic inflammatory symptoms with a 5-year survival of less than 50%. PMF affects both young and older adults and can evolve into acute leukemia in >15% of cases. Mutations within the gene encoding calreticulin (CALR) are the second most common genetic aberration associated with PMF, observed in 70% of non-JAK2^{V617F} cases.

Most patients with non-JAK2 mutated essential thrombocythemia also carry a mutation in the CALR gene. Studies have shown that CALR-mutated cases in essential thrombocythemia are associated with younger age, higher platelet counts, lower erythrocyte counts, lower leukocyte counts, and lower haemoglobin levels compared with JAK2-mutated cases.

Importantly, primary myelofibrosis patients with CALR mutations do not effectively respond to JAK inhibitor therapy and to date no CALR-specific therapy has been developed.

Virtually all CALR mutations identified in myeloproliferative diseases are small insertions or deletions clustered within exon 9, leading to frameshift mutations in the protein. The Type-1 variant is a deletion variant and the Type-2 variant is an insertion variant. The frameshift mutations lead to a neo-epitope peptide sequence which is thought to directly or indirectly activate the thrombopoietin receptor (TpoR) by a poorly defined mechanism that is dependent on glycan binding sites. There are subtle differences in the biochemistry between Type 1 and Type 2 CALR mutations.

Accordingly, there is a need to develop new or improved therapeutics for treating PMF and other myeloproliferative studies, and in particular to develop a therapeutic for CALR myeloprofilerative disorders which would have activity against both Type 1 and Type 2 CALR mutations with minimal to no effect on normal haematopoiesis.

### SUMMARY

The present disclosure relates to antibodies to calreticulin, methods for preventing and/or treating myeloproliferative diseases using antibodies, and chimeric antigen receptors having extracellular domains based on the antibodies.

The present disclosure is based on the development of calreticulin (CALR) antibodies directed against a common sequence encoded by both insertion and deletion mutations of the protein, which contributes to the development of some myeloproliferative disease. Some of the antibodies developed have the property of disrupting the binding of mutant CALR dimers to the thrombopoietin receptor, and have the ability to block the role of mutant CALR in JAK-STAT signalling, TPO-independent proliferation and megakaryocyte differentiation. Importantly, it was also determined that some of the antibodies have the ability to inhibit proliferation of patient samples with both insertion and deletion CALR mutations, but not patient samples having JAK2 V617F. The antibodies also prolonged survival in xenografted bone marrow models of mutant CALR-dependent myeloproliferation. The studies described herein confirm the possibility of a new therapeutic approach to target a disease driven by recurrent somatic mutations that would normally be considered undruggable, by using agents that disrupt binding mutant CALR dimers to the thrombopoietin receptor.

Certain embodiments of the present disclosure provide a method of preventing and/or treating a myeloproliferative disorder in a subject, the myeloproliferative disorder associated with the presence of a frameshift mutation in the CALR gene of a cell involved in the myeloproliferative disorder, the method comprising exposing the subject, and/or cells involved in the myeloproliferative disorder, to an effective amount of an immunological agent that binds to an epitope in the CALR protein arising from the frameshift mutation in the CALR gene.

Certain embodiments of the present disclosure provide a method of preventing and/or treating a myeloproliferative disorder in a subject, the disorder associated with the presence of a frameshift mutation in the CALR gene of a cell involved in the myeloproliferative disorder, the method comprising inhibiting binding of dimers of a CALR protein arising from the frameshift mutation in the CALR gene to the thrombopoietin receptor in cells involved with the myeloproliferative disorder.

Certain embodiments of the present disclosure provide a method of preventing and/or treating a myeloproliferative disorder in a subject, the myeloproliferative disorder associated with the presence of a frameshift mutation in the CALR gene, the method comprising exposing the subject, and/or cells involved in the myeloproliferative disorder, to an effective amount of an immunological agent that binds to an epitope in the CALR protein arising from the frameshift mutation in the CALR gene.

Certain embodiments of the present disclosure provide a method of inhibiting JAK-STAT signalling in a megakaryocyte and/or a progenitor cell thereof, the megakaryocyte and/or the progenitor cell comprising a frameshift mutation in the CALR gene, the method comprising inhibiting binding of dimers of a CALR protein arising from the frameshift mutation in the CALR gene to the thrombopoietin receptor in the megakaryocyte and/or the progenitor cells.

Certain embodiments of the present disclosure provide a method of inhibiting JAK-STAT signalling in a megakaryocyte and/or a progenitor cell thereof, the megakaryocyte and/or the progenitor cell comprising a frameshift mutation in the CALR gene, the method comprising exposing the megakaryocyte and/or the progenitor cells to an effective amount of an immunological agent that binds to an epitope in the CALR protein arising from the frameshift mutation in the CALR gene.

Certain embodiments of the present disclosure provide a method of inhibiting thrombopoietin-independent proliferation and/or differentiation of a megakaryocyte cell and/or a progenitor cell thereof, the megakaryocyte and/or the progenitor cell comprising a frameshift mutation in the CALR gene, the method comprising inhibiting binding of dimers of a CALR protein arising from the frameshift mutation in the CALR gene to the thrombopoietin receptor in the megakaryocyte and/or the progenitor cells.

Certain embodiments of the present disclosure provide a method of inhibiting thrombopoietin-independent proliferation and/or differentiation of a megakaryocyte cell and/or a progenitor cell thereof, the megakaryocyte cell and/or the progenitor cell comprising a frameshift mutation in the CALR gene, the method comprising exposing the megakaryocyte cell and/or the progenitor cell to an effective amount of an immunological agent that binds to an epitope in the CALR protein arising from the frameshift mutation in the CALR gene.

Certain embodiments of the present disclosure provide an immunological agent that binds to an epitope in a CALR protein arising from a frameshift mutation to the thrombopoietin receptor.

Certain embodiments of the present disclosure provide an immunological agent that inhibits binding of dimers of a CALR protein arising from a frameshift mutation to the thrombopoietin receptor.

Certain embodiments of the present disclosure provide use of an immunological agent as described herein for preventing and/or treating a myeloproliferative disorder.

Certain embodiments of the present disclosure provide an isolated polypeptide consisting of 10 to 50 amino acids, the polypeptide comprising all or part of an amino acid sequence having at least 90% sequence identity with SEQ ID NO: 1.

Certain embodiments of the present disclosure provide use of a polypeptide as described herein as an immunogen.

Certain embodiments of the present disclosure provide a method of producing an antibody to a frameshift mutant of the CALR protein, the method comprising raising an antibody in an animal to a polypeptide as described herein.

Certain embodiments of the present disclosure provide an antibody produced by a method as described herein.

Certain embodiments of the present disclosure provide an antibody, or an antigen binding part thereof, that binds to an epitope in the CALR protein arising from a frameshift mutation in the CALR gene.

Certain embodiments of the present disclosure provide an antibody, or an antigen binding part thereof, that inhibits binding of a dimer of a frameshift mutant CALR protein to the thrombopoietin receptor.

Certain embodiments of the present disclosure provide an antibody, or an antigen binding part thereof, comprising:
an antibody heavy chain variable region comprising the complementary determining regions of a CDR1 of SEQ ID NO: 18, a CDR2 of SEQ ID NO:22 and a CDR3 of SEQ ID NO:26, and
an antibody light chain variable region comprising the complementary determining regions of a CDR1 of SEQ ID NO:27, a CDR2 of SEQ ID NO:30 and a CDR3 of SEQ ID NO:31.

Certain embodiments of the present disclosure provide an antibody, or an antigen binding part thereof, comprising:
an antibody heavy chain variable region comprising the complementary determining regions of a CDR1 of SEQ ID NO:16 or 17, a CDR2 of SEQ ID NO: 19, 20 or 21 and a CDR3 of SEQ ID NO:23, 24 or 25, and
an antibody light chain variable region comprising the complementary determining regions of a CDR1 of SEQ ID NO:27, a CDR2 of SEQ ID NO:28 or 29, and a CDR3 of SEQ ID NO:31.

Certain embodiments of the present disclosure provide an antibody, or an antigen binding part thereof, comprising:
an antibody heavy chain variable region comprising the complementary determining regions of SEQ ID NO: 5 and an antibody light chain variable region comprising complementary determining regions of SEQ ID NO: 7 (4D7).

Certain embodiments of the present disclosure provide an antibody, or an antigen binding part thereof, comprising:
an antibody heavy chain variable region comprising the complementary determining regions of SEQ ID NO: 9 and an antibody light chain variable region comprising the complementary determining regions of SEQ ID NO: 11 (2D2).

Certain embodiments of the present disclosure provide an antibody, or an antigen binding part thereof, comprising:
an antibody heavy chain variable region comprising the complementary determining regions of SEQ ID NO: 13 and an antibody light chain variable region comprising the complementary determining regions of SEQ ID NO: 15 (9H11).

Certain embodiments of the present disclosure provide a hybridoma expressing an antibody as described herein.

Certain embodiments of the present disclosure provide a chimeric antigen receptor comprising an extracellular domain having a binding domain that binds to an epitope in a CALR protein arising from a frameshift mutation in the CALR gene.

Certain embodiments of the present disclosure provide a chimeric antigen receptor comprising an extracellular domain having a binding domain that inhibits binding of a dimer of a frameshift mutant CALR protein to the thrombopoietin receptor.

Certain embodiments of the present disclosure provide a chimeric antigen receptor comprising an extracellular domain comprising a binding domain that binds to SEQ ID NO:1, a transmembrane domain, and an intracellular signalling domain that activates a cellular function.

Certain embodiments of the present disclosure provide a chimeric antigen receptor comprising an extracellular domain comprising a binding domain comprising an antibody heavy chain variable region and an antibody light chain variable region, wherein the antibody heavy chain variable region comprises the complementary determining regions of a CDR1 of SEQ ID NO: 18, a CDR2 of SEQ ID NO:22 and a CDR3 of SEQ ID NO: 26, and the antibody light chain variable region comprises the complementary determining regions of a CDR1 of SEQ ID NO:27, a CDR2 of SEQ ID NO:30 and a CDR3 of SEQ ID NO:31.

Certain embodiments of the present disclosure provide a chimeric antigen receptor comprising an extracellular domain comprising a binding domain comprising:
an antibody heavy chain variable region comprising the complementary determining regions of a CDR1 of SEQ ID NO: 18, a CDR2 of SEQ ID NO:22 and a CDR3 of SEQ ID NO:26, and
an antibody light chain variable region comprising the complementary determining regions of a CDR1 of SEQ ID NO:27, a CDR2 of SEQ ID NO:30 and a CDR3 of SEQ ID NO:31:
or a functional variant having at least 90% sequence identity to any one or more of the aforementioned complementary determining regions.

Certain embodiments of the present disclosure provide a chimeric receptor antigen comprising an extracellular domain comprising a binding domain comprising:
an antibody heavy chain variable region comprising the complementary determining regions of a CDR1 of SEQ ID NO:16 or 17, a CDR2 of SEQ ID NO: 19, 20 or 21, and a CDR3 of SEQ ID NO:23, 24 or 25, and
an antibody light chain variable region comprising the complementary determining regions of a CDR1 of SEQ ID NO:27, a CDR2 of SEQ ID NO:28 or 29, and a CDR3 of SEQ ID NO:31.

Certain embodiments of the present disclosure provide a nucleic acid molecule comprising a nucleotide sequence encoding an antibody, or an antigen binding part thereof as described herein or a chimeric antigen receptor as described herein.

Certain embodiments of the present disclosure provide a vector comprising a nucleic acid as described herein.

Certain embodiments of the present disclosure provide a cell comprising a chimeric antigen receptor as described herein, a nucleic acid as described herein or a vector as described herein.

Certain embodiments of the present disclosure provide a method of killing a target cell expressing a mutant CALR protein arising from a frameshift mutation in the CALR gene, the method comprising exposing the target cell to a cell expressing the chimeric antigen receptor as described herein.

Certain embodiments of the present disclosure provide a method of preventing or treating a CALR mutant myeloproliferative disorder in a subject, the method comprising exposing the subject to a cell as described herein.

Certain embodiments of the present disclosure provide a pharmaceutical composition comprising a cell as described herein, a nucleic acid as described herein, or a vector as described herein.

Certain embodiments of the present disclosure provide a method of preventing or treating a subject for a CALR mutant myeloproliferative disorder, the method comprising:
assessing the subject for the presence of a frameshift mutant in the CALR gene in cells involved in the myeloproliferative disorder; and treating a subject identified to have the frameshift mutation in the CALR gene with an effective amount of an immunological agent that binds to an epitope in the CALR protein arising from the frameshift mutation in the CALR gene.

Certain embodiments of the present disclosure provide a method of preventing and/or treating a subject for a CALR mutant myeloproliferative disorder, the method comprising:
assessing the subject for the presence of a frameshift mutant in the CALR gene in cells involved in the myeloproliferative disorder; and inhibiting binding of dimers of a CALR protein arising from the frameshift mutation in the CALR gene to the thrombopoietin receptor in cells in the subject involved with the myeloproliferative disorder.

Certain embodiments of the present disclosure provide a method of preventing and/or treating a myeloproliferative disorder in a subject, the disorder associated with the presence of a frameshift mutation in the CALR gene of a cell involved in the myeloproliferative disorder, the method comprising exposing the subject, and/or cells involved in the myeloproliferative disorder, to an effective amount of an immunological agent as described herein, an antibody and/or an antigen binding part thereof as described herein, a chimeric antigen receptor as described herein, a nucleic acid as described herein. a vector as described herein, a cell as described herein, or a pharmaceutical composition as described herein.

Certain embodiments of the present disclosure provide a method of inhibiting JAK-STAT signalling in a megarokaryocyte cell and/or a progenitor cell thereof, the megakaryocyte cell and/or the progenitor cell comprising a frameshift mutation in the CALR gene, the method comprising exposing the megakaryocyte cell and/or the progenitor cells to an effective amount of an immunological agent as described herein, an antibody and/or an antigen binding part thereof as described herein, a chimeric antigen receptor as described herein, a nucleic acid as described herein. a vector as described herein, a cell as described herein, or a pharmaceutical composition as described herein.

Certain embodiments of the present disclosure provide a method of inhibiting thrombopoietin-independent proliferation and/or differentiation of a megakaryocyte cell and/or a progenitor cell thereof, the megakaryocyte cell and/or the progenitor cell comprising a frameshift mutation in the CALR gene, the method comprising exposing the megakaryocyte cell and/or the progenitor cell to an effective amount of an immunological agent as described herein, an antibody and/or an antigen binding part thereof as described herein, a chimeric antigen receptor as described herein, a nucleic acid as described herein. a vector as described herein, a cell as described herein, or a pharmaceutical composition as described herein. Other embodiments are described herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows anti-mutant calreticulin antibody binds to cell surface and inhibits TPO-independent proliferation A. Schematic showing wild type C-terminal calreticulin protein sequence and neoepitope sequences for 52 bp deletion or 5 bp insertion and peptide sequence used for immunization. B. Diluting concentrations of 4D7 to bound full-length 30 amino acid peptide compared to scrambled, C-terminal and N-terminal 11 amino acid peptides as shown in A (n=3). C. Scatchard analysis showing dissociation constant of 4D7 using I¹²⁵-labelled and unlabelled 4D7 bound to full-length 30 amino acid peptide (n=3). D. Histogram showing fluorescence intensity of 4D7 conjugated to phycoerythrin at a 4 μg/mL TF-1 TpoR CALR^{del 61} vs TF-1 TpoR compared to unstained. E. TF-1 TpoR cells with endogenous wild type CALR cultured in the presence of TPO and 2, 10 or 20 μg/mL 4D7 anti-mutant CALR antibody or 20 μg/mL control IgG antibody (n=3). F. Proliferation curves of factor-independent TF-1 TpoR CALR^{del61} cells cultured with 2, 10 or 20 μg/mL 4D7 or 20 μg/mL of control IgG antibody (n=3). G Proliferation curves of factor-independent TF-1 TpoR CALR^{del52} cells cultured with 10 or 20 g/mL 4D7 or 20 μg/mL of control IgG antibody (n=3). Data information: For panels E-G, bars represent standard error of the mean summarizing 3 independent experiments performed in triplicate, with a one-way ANOVA with Bonferroni correction used to determine statistical significance.

FIG. 2 shows understanding the mechanism of action of the 4D7 monoclonal antibody. A. Cell extracts blotted for phospho-STAT5, total STAT5, phospho-ERK, total ERK and actin from TF-1 TpoR cells after incubation with 10 or 20 μg/mL 4D7 or IgG for 4 or 8 hours, in presence of TPO. The last line indicates TPO withdrawal. B. Similar experiment using TPO-independent TF-1 TpoR CALR^{del61} cells. C. Similar experiment using TPO-independent TF-1 TpoR CALR^{del52} cells at 8 hours. D. Fraction of apoptotic sub-Go population of TF-1 TpoR CALR^{del61} cells after 48 hours of 4D7 or IgG treatment (n=3). E. Western blot showing caspase 3 cleavage occurring within 24 hours of TPO withdrawal in TPO dependent TF-1 TpoR cells. An increase in cleaved caspase 3 is observed after 48 hours of treatment with 20 μg/mL 4D7 in TF-1 TpoR CALR^{del61} and TF-1 TpoR CALR^{del52} cells. F. Western blot of TpoR immunoprecipitation under non-reducing conditions showing associated CALR 50 kDa monomers and 100 kDa dimers (arrowheads) present only in TF-1 TpoR CALR^{del61} disrupted by 8 hour treatment with 20 μg/mL 4D7 but not PBS or 20 μg/mL IgG. CALR monomers & dimers are detectable by polyclonal anti-wild type CALR or anti-mutant CALR monoclonal antibodies. Arrowheads, detected mutant CALR protein: asterisk, non-specific bands. G. Proposed model for the mechanism of action of 4D7 on mutant CALR. In the active form, mutant CALR dimers bound to TpoR form a constitutively activate receptor complex which is disrupted by 4D7 with blockade in STAT and ERK signalling. (Adapted from Masubuchi et al. (2020), Rivera et al (2021)). Data information: For panel D, bars represent standard deviation for 3 replicates, normalised to IgG, with a Students unpaired t-test used to determine statistical significance.

FIG. 3 shows 4D7 monoclonal antibody specifically inhibits primary megakaryocyte differentiation of mutated CALR myelofibrosis samples. A. PCR amplification of CALR exon 9 from patients confirming heterozygous del52 mutation in sorted CD34+ cells obtained from CALR mutated myelofibrosis samples. B. Graph showing the decreased fold change of CD41+CD61+megakaryocytes cultured in 4D7 compared to IgG. FACS-sorted CD34+ from myelofibrosis patients with CALR or JAK2 mutation were cultured over 12 days without TPO in the presence of SCF, IL-6 and IL-9. Black columns show JAK2^{V617F} mutation positive samples (n=4). C. Number of CD41+megakaryocytes derived from isolated CD34+progenitors from myelofibrosis patients. Number of CD41/CD61+ cells counted on day 12 using trypan blue exclusion (n=1). D. Summary of fold change reduction of CD41/CD61+megakaryocytes by 4D7 in all tested CALR mutated patient samples compared to CALR wild type, normalised to IgG (n=11). E Number of CD41+megakaryocyte colonies from patient samples after 4D7 treatment. CD34+ from patients with myelofibrosis with CALR^{del52} or CALR^{ins5} were plated on collagen-based matrix in presence of 20 μg/mL 4D7 or IgG control (n=3). F. Representative micrographs showing CD41+colonies in pink and CD41-colonies in blue after treatment with 4D7 or IgG at 100 ×or 40× magnification. G. Summary of fold change reduction of CD41+megakaryocytes in CALR mutated samples cultured MegaCult treated with 4D7 compared to IgG (n=4). H. Number of megakaryocyte colony forming units grouped according colony cell number after 4D7 treatment in a mutant CALR^{ins5} sample (n=3). Data information: Bars represent standard error of means in D and G. Unpaired students t-test used to determine statistical significance. Bars represent standard deviations in B. E and H.

FIG. 4 shows 4D7 has no effect on normal progenitor cells and shows activity against ruxolitinib-resistant cells without hematological toxicity. A. Graph showing effect of ruxolitinib on megakaryocyte differentiation at 50 and 100 nM in healthy CD34+ cells (n=4). B. Effect of 4D7 on TPO-dependent megakaryocyte differentiation of healthy CD34+ cells cultured with 20 μg/mL 4D7 or IgG control (n=4). C. Total numbers of hematopoietic colonies plated in MethoCult from healthy cord blood after treatment with 4D7 or IgG. CFU-GM, colony forming unit-granulocyte macrophage: BFU-E, blast forming unit-erythroid: CFU-GEMM, colony forming unit granulocyte, erythroid, monocyte, megakaryocyte (n=3).D. CD41+megakaryocyte colonies from healthy CD34+ cells observed on collagen matrix after 4D7 treatment. Colonies were cultured in TPO, SCF. IL-9 and IL-3. E. Number of megakaryocyte colony forming units observed in MegaCult assay after treatment of healthy cord blood CD34+ cells with either IgG or 4D7 (n=3). F. Western blot showing signalling in ruxolitinib-resistant TF-1 TpoR CALR^{del61} compared to ruxolitinib sensitive TF-1 TpoR CALR^{del61} cells after treatment with 100 nM ruxolitinib for 16 hours or 30 minutes and blotted for phospho-STAT5, total STAT5, phospho-ERK, total ERK and actin as indicated. Ruxolitinib-sensitive cells were non-viable after 16 hours of treatment. G. Comparison of cell growth after DMSO, 20 μg/mL 4D7, IgG or 100 nM ruxolitinib treatment over 4 days of ruxolitinib-resistant TF-1 TpoR CALR^{del61}. Cells counted using Trypan Blue exclusion with replicate wells (n=3). H. Number of colonies observed from cells plated in MethoCult following 72 hours of treatment with DMSO, 4D7, IgG or 100 nM ruxolitinib performed in ruxolitinib-resistant TF-1 TpoR CALR^{del61} cells (n=3). Data information: A, B, C and E were performed 3× independently and error bars indicate standard error of mean. G and H were performed 3 times with representative experiments shown. Error bars represent standard deviation. All P values are unpaired Student's unpaired t-test.

FIG. 5 shows 4D7 blocks mutant CALR-dependent myeloid proliferation in vivo and prolongs survival. A. Illustration showing bone marrow NSG engraftment model with TPO-independent TF-1 TpoR CALR^{del61} cells treated with 4D7 or IgG control twice weekly, starting 7 days after engraftment via intraperitoneal injection and final measurements taken from euthanized mice. B. Pharmacokinetic measurements of serum level of 4D7 in mice after intraperitoneal injection at 1, 24, 48, 72 and 110 hours since administration. C. Percentage of human CD33+myeloid cells measured in peripheral blood at 3 weeks post-tail vein engraftment (n=5 mice per treatment). D. Kaplan-Meier survival curve of bone marrow engraftment model showing improved survival of mice treated with 4D7 compared with IgG control commencing I week after tail vein injection (n=5 mice per treatment). E. Mean tumour volume at 21 days after sub-cutaneous injection of TF-1 TpoR CALR^{del61} cells following treatment with 4D7 or IgG. Cells were pre-treated with 4D7 or IgG control 1 hour prior to injection and treatment continued twice weekly at 12.5 mg/kg until euthanasia (n=3 mice per treatment). F. Kaplan-Meier survival curve of chloroma mice treated with 4D7 or IgG until humane killing due to tumour diameter >30 mm or ulceration. (n=3 mice per treatment). G. Kaplan-Meier survival curve of NSG mice engrafted with ruxolitinib-resistant TF-1 TpoR CALR^{del61} treated with 4D7 or IgG 12.5 mg/kg twice weekly (n=5 and 6 mice for IgG and 4D7 respectively). Data information: For C and E, a student's unpaired t-test was used to determine statistical significance. For all survival curves the log-rank Mantel-Cox test P value is shown.

FIG. 6 shows determination of efficacy of 4D7 antibody as a potential therapeutic. A. Western blot screening of various mutant CALR antibody clones produced from hybridomas in TF-1 TpoR and TF-1 TpoR CALR^{del61}. Varying levels of intensity can be observed, compared to commercial Dianova monoclonal mutant CALR antibody. 4D7 is able to detect mutant CALR protein in CALR^{del61} cells but not in CALR wild type TF-1 cells. B. Replicate Scatchard analyses of 4D7 using I¹²⁵-labelled and unlabelled 4D7 bound to full-length peptide (n=3). C. TF-1 cells expressing TpoR and CALR^{del61} or CALR^{del52} demonstrate factor independence in absence of TPO. Cells were cultured in the presence or absence of 10 ng/ml TPO (n=3). D. Paracrine CALR-mutant protein is not sufficient to maintain TPO sensitive cells in culture. TF1 TpoR and TF-1 TpoR CALR^{del61} cells were seeded at the same density and cultured in the presence or absence of TPO. Cells were separated by semi-permeable membrane in a horizontal co-culture system. Cell populations on either side of the membrane were counted every 24 hours over 4 days in triplicate and representative images were taken on day 4. Exogenous CALR secreted by TF-1 TpoR CALR^{del61} was unable to assist growth of factor dependent TF-1 TopR cells (n=3). E. Histogram overlays showing fluorescence intensity of unstained and PE conjugated IgG2a isotype control in TF-1, TF-1 TpoR and TF-1 TpoR CALR^{del61} compared to 4D7 conjugated to PE.

FIG. 7 shows biological Specificity of 4D7. A. Cytokine dependent TF-1 TpoR cells with an overexpression of WT CALR cultured in the absence of TPO, 10 ng/ml hTPO and 10 or 20 μg/mL 4D7 anti-mutant CALR antibody or 20 μg/mL control IgG antibody for 5 days and the number of trypan blue negative cells were counted every 24 hours (n=3). B. Cytokine dependent TF-1 CALR^{del52} cells, lacking TpoR were cultured in the presence of 2 ng/mL GM-CSF and 10 or 20 μg/mL 4D7 or 20 μg/mL control IgG antibody (n=3). C. Cytokine dependent TF-1 CALR^{del61} cells, lacking TpoR were cultured in the presence of 2 ng/ml GM-CSF and 10 or 20 μg/mL 4D7 or 20 μg/mL control IgG antibody (n=3). D. MARIMO cells from which the CALR^{del61} mutation was originally amplified were cultured in the presence of 2, 10 or 20 μg/mL 4D7 or 20 μg/mL control IgG antibody (n=3). E. Cytokine independent SET2 cells which harbour the pathogenic JAK2^{V617F} mutation were cultured in the presence of 2, 10 or 20 μg/mL 4D7 or 20 μg/mL control IgG antibody (n=3). F. Cytokine independent TF-1 PTPN11^{E76K} cells were cultured in the presence of 10 or 20 μg/mL 4D7 or 20 μg/mL control IgG antibody (n=3).

FIG. 8 shows proliferation curves of factor-independent TF-1 TpoR CALR^{del61} cells cultured with 2D2 or 9H11. A. Proliferation curves of factor-independent TF-1 TpoR CALR^{del61} cells cultured with 5 or 20 μg/mL of 2D2 or 20 μg/mL of control IgG antibody (n=3 biological replicates). B. Proliferation curves of factor-independent TF-1 TpoR CALR^{del61} cells cultured with 5 or 20 μg/mL of 9H11 or 20 μg/mL of control IgG antibody (n=3 biological replicates).

FIG. 9 shows monoclonal antibody blocks STAT1, 3 signalling and TpoR phosphorylation. A. Cell extracts blotted for phospho-STAT1, total STAT1, phospho-STAT3, total STAT3 and actin from TF-1 TpoR cells after incubation with 10 or 20 μg/mL 4D7 or IgG for 4 or 8 hours as indicated. B. Similar experiment using TPO-independent TF-1 TpoR CALR^{del61} cells at 4 and 8 hours. C. Similar experiment shown using TPO-independent TF-1 TpoR CALR^{del52} cells, 8 hours. D. Flow cytometry analysis for cell cycle distribution of TF-1 TpoR CALR^{del61} cells exposed to 20 μg/mL IgG or 4D7 for 48 hours. Cells were harvested and fixed and stained with propidium iodide and their DNA contents were analysed. Results from one representative experiment shown. Percentages of cells in Sub Go, G1. S and G2/M cycle indicated. E. Western blot showing specific co-immunoprecipitation of mutant CALR^{del61} cells with TpoR anti-FLAG antibody under reducing conditions detected by total and mutant specific CALR antibodies. Arrowheads represent detected CALR protein. F. Western blot showing decreased TpoR phosphorylation in a TpoR immunoprecipitated after 8 hours of 20 μg/mL 4D7 treatment compared to PBS or IgG control in TF-1 TpoR CALR^{del61}.

FIG. 10 shows the strategy for the sorting of PMF primary CD34+ cells. A. Western blot of TpoR immunoprecipitation under non-reducing conditions showing associated CALR 50 kDa monomers and 100 kDa dimers (arrowheads) present only in TF-1 TpoR CALR^{del52} disrupted by 8 hour treatment with 20 μg/mL 4D7 but not PBS or 20 μg/mL IgG. CALR monomers & dimers are detectable by polyclonal anti-wild type CALR or anti-mutant CALR monoclonal antibodies. Arrowheads, detected mutant CALR protein: asterisk, non-specific bands. B. Peripheral blood mononuclear cells from PMF samples were thawed and stained for CD34, CD14, CD19 and CD3 prior to FACS sorting. Each population was collected and purity was verified prior to proceeding with any further analysis. PCR amplification of CALR exon 9 was carried out to confirm mutational status of CD34+ cells which were utilised in megakaryocyte differentiation assays and colony forming assays in the presence of 4D7 or IgG. C. Representative flow cytometry plots for determination of CD41+/61+ populations from liquid culture assay from one PMF patient. Beads shown in the upper left panel with high SSC-A. Live cell population shown in hexagon gate (top panel). CD41/61+ population gates shown in lower panel with % CD41/61+ cells indicated for unstained, IgG and 4D7 treated cells over 12 days. The number of CD41/61+ cells to bead ratio used to enumerate effect of 4D7 on megakaryopoesis.

FIG. 11 shows the clinical details for all patients.

FIG. 12 shows the effect of 4D7 antibody on TPO receptor biology. A. Proliferation curves of factor-independent TF-1 TpoR CALR^{del61} cells cultured with 10 or 20 μg/ml 4D7 or 20 μg/ml of control IgG antibody in presence or absence of 10 ng/ml TPO (n=3 biological replicates with three technical replicates). B. Number of CD41+megakaryocyte colonies CALR^{del52} patient after 4D7 treatment in the presence of TPO. Samples were seeded in a collagen-based matrix in presence of 20 μg/ml 4D7 or IgG control with 50 ng/ml TPO (n=2 patient samples with two technical replicates). C. Representative micrographs showing CD41+colonies in light grey and CD41-colonies in blue after treatment with IgG or 4D7 in absence or presence of 50 ng/ml TPO. Scale bar indicates 100 μm. Data information. Error bars represent standard error of the mean in (A) and standard deviation (B). Unpaired Student's t-test used to determine statistical significance in (B). * P=0.05-0.01, ** P=0.01-0.001, *** P=0.001-0.0001.

FIG. 13 shows the general workflow for antibody sequencing.

FIG. 14 shows the sequencing results of the VH and VL regions of antibody 4D7.

FIG. 15 shows the VH and VL protein sequence annotated to highlight framework regions and complementary determining regions for antibody 4D7.

FIG. 16 shows the sequencing results of the VH and VL regions of antibody 2D2.

FIG. 17 shows the VH and VL protein sequence annotated to highlight framework regions and complementary determining regions for antibody 2D2.

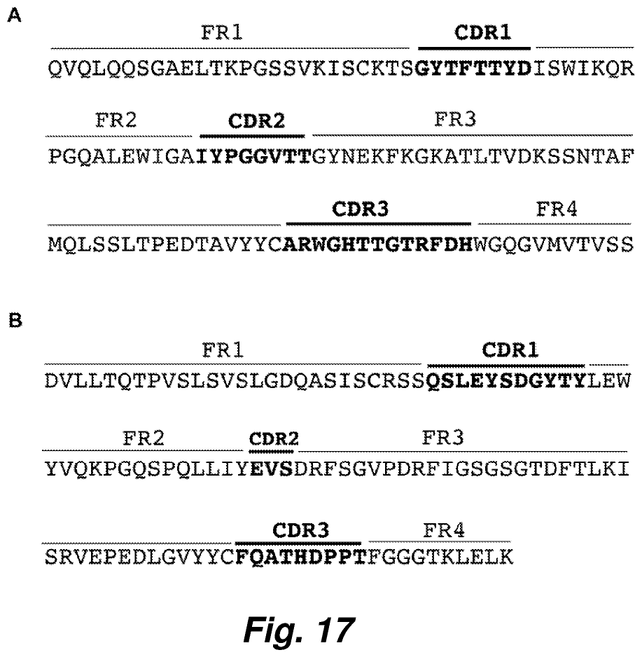

FIG. 18 shows the sequencing results of the VH and VL regions of antibody 9H11.

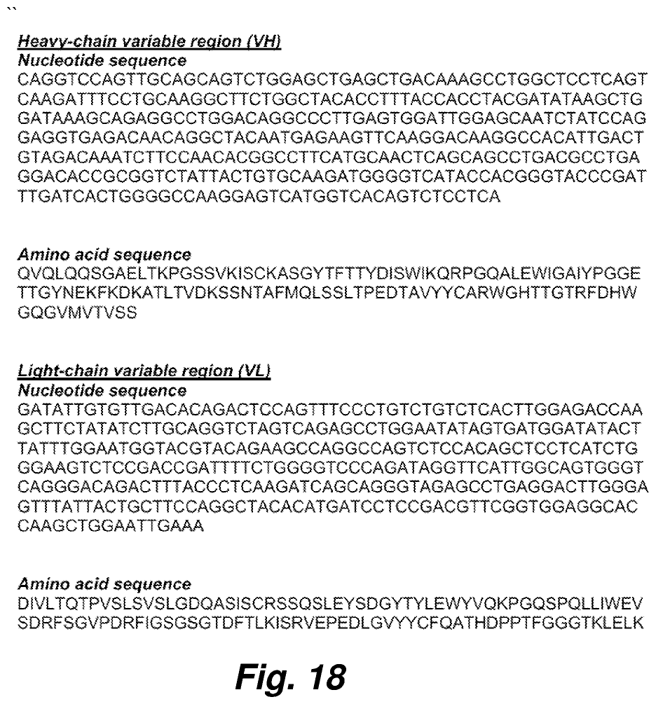

FIG. 19 shows the VH and VL amino acid sequences annotated to highlight framework regions and complementary determining regions for antibody 9H11.

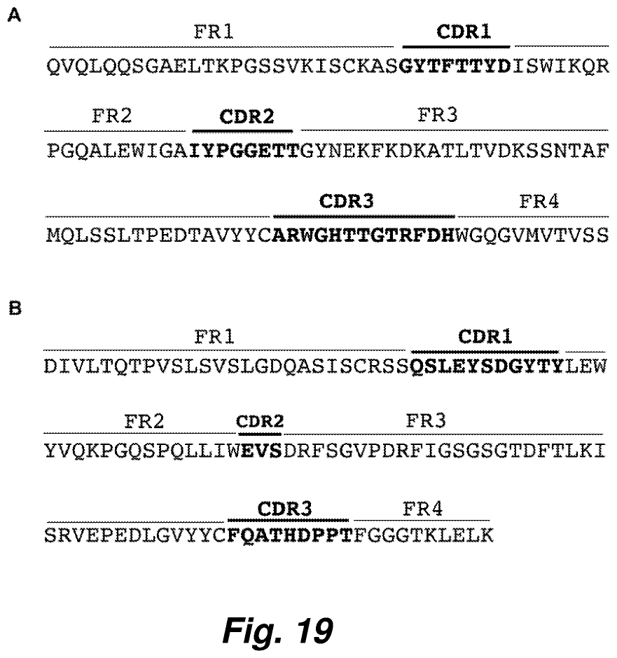

FIG. 20 shows the amino acid sequences of the VH CDR1, CDR2 and CDR3 regions for 4D7, 2D2 and 9H11, and the consensus sequences for each of those regions.

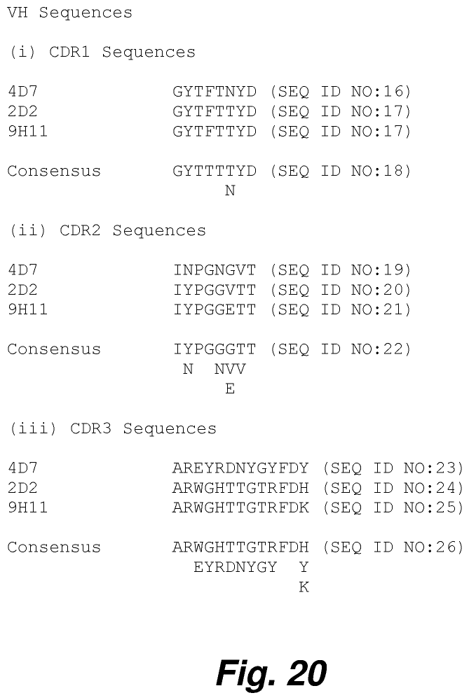

FIG. 21 shows the amino acid sequences of the VL CDRI, CDR2 and CDR3 regions for 4D7, 2D2 and 9H11, and the consensus sequences for each of those regions.

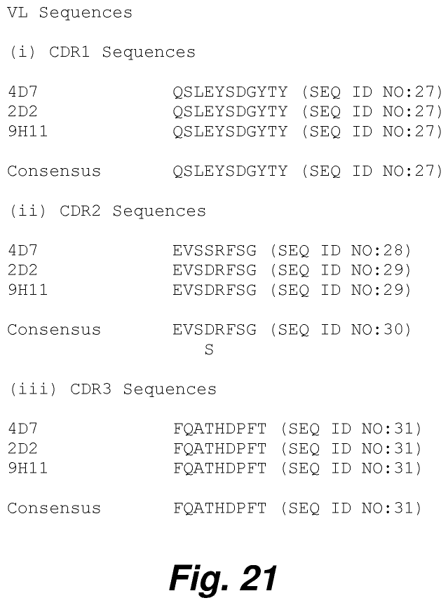

### DETAILED DESCRIPTION

The present disclosure relates to methods for preventing and/or treating myeloproliferative diseases, antibodies and other immunological agents to calreticulin, methods for preventing and/or treating myeloproliferative diseases using the antibodies and immunological agents, and chimeric antigen receptors having extracellular domains based on the antibodies.

Certain embodiments of the present disclosure provide a method of preventing and/or treating a myeloproliferative disorder.

In certain embodiments, the present disclosure provides a method of preventing and/or treating a myeloproliferative disorder in a subject, the myeloproliferative disorder associated with the presence of a frameshift mutation in the CALR gene of a cell involved in the myeloproliferative disorder, the method comprising exposing the subject, and/or cells involved in the myeloproliferative disorder, to an effective amount of an immunological agent that binds to an epitope in the CALR protein arising from the frameshift mutation in the CALR gene.

The term “disorder” as used herein in relation to a medical or veterinary disorder and refers to a condition, disease, an illness, a state, a precondition, or other type of physiologic disorder.

In certain embodiments, the subject is a human subject. Veterinary applications of the present disclosure in animals are contemplated, as are use of the various embodiments of the present disclosure for research purposes.

In certain embodiments, the subject is suffering from a myeloproliferative disorder. In certain embodiments, the subject is susceptible to a myeloproliferative disorder.

In certain embodiments, the cells involved in the myeloproliferative disorder are present in vivo in a subject, or are present ex vivo, for example for reintroduction into the subject. Ex vivo cells may be autologous cells, syngeneic cells or allogeneic cells. Methods for obtaining ex vivo cells are known in the art, as are methods for treating ex vivo cells and their reintroduction into a subject.

The term “preventing”, and related terms such as “prevention” and “prevent”, as used herein refers to obtaining a desired therapeutic and/or physiologic effect in terms of arresting or suppressing the appearance of one or more symptoms or other characteristics in the subject.

The term “treatment”, and related terms such as “treating” and “treat” as used herein, refer to obtaining a desired pharmacologic and/or physiologic effect in terms of improving the condition of the subject, ameliorating, arresting, managing, suppressing, relieving and/or slowing the progression of one or more symptoms in the subject, a partial or complete stabilization of the subject, a regression of one or more symptoms, or a cure in the subject.

In certain embodiments, the myeloproliferative disorder comprises primary myelofibrosis, thrombocythemia, or secondary acute myeloid leukemia. Other myeloproliferative disorders are contemplated. Methods for diagnosing subjects with a myeloproliferative disorder are known in the art.

The NCBI accession number for the human CALR gene is HGBC:1455. Orthologs of the gene are known, or may be identified by a method known in the art. Methods for identifying whether a subject carries a CALR mutation are known in the art, and typically involve taking a blood sample or a bone marrow biopsy and analysing the sample by a DNA diagnostic or sequencing method.

The term “epitope” as used herein refers to a part of an antigen that is recognized by the immune system, and is the specific part of the antigen to which an antibody binds.

In this regard, that part of the CALR protein that is produced as a result of a frameshift mutation may also be referred to herein as a “neo-epitope”. A CALR protein having a frameshift mutation may also be referred to herein in some embodiments to as a “mutant CALR protein”.

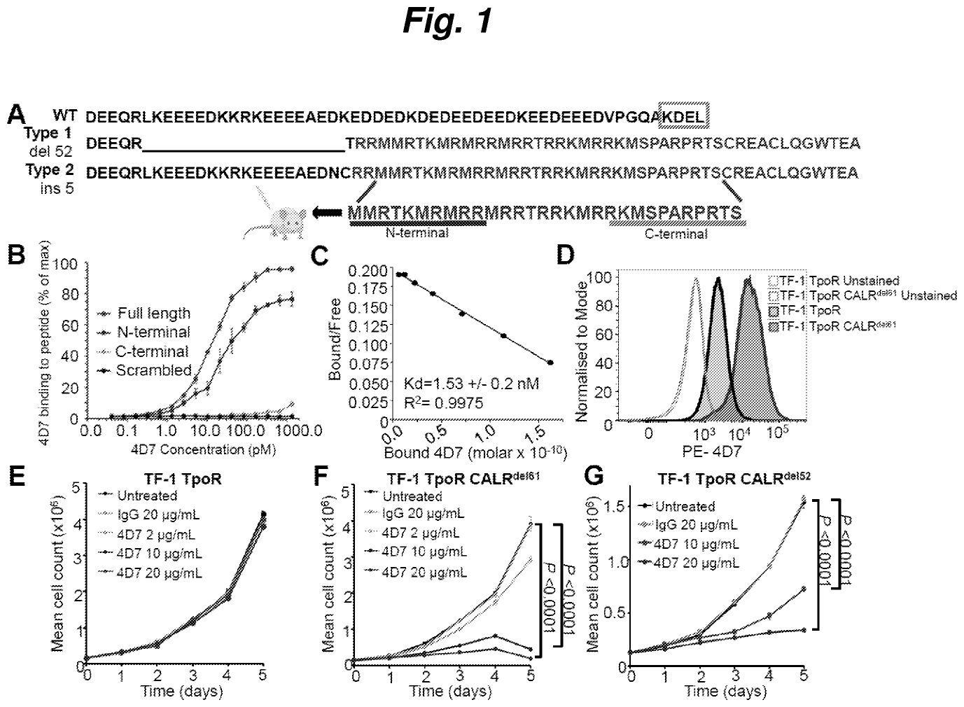

In certain embodiments, the frameshift mutation is a +1 frameshift mutation. In certain embodiments, the frameshift mutation comprises a frameshift mutation in exon 9 of the CALR gene. In certain embodiments, the frameshift mutation comprises a CALR^{del52} frameshift mutation or a CALR^{ins5} frameshift mutation, as shown in FIG. 1A. Other frameshift mutations are contemplated.

In certain embodiments, the myeloproliferative disorder is a ruxolitinib resistant disorder. In certain embodiments, the myeloproliferative disorder is a ruxolitinib resistant primary myelofibrosis. Ruxolitinib is a tyrosine kinase inhibitor which inhibits JAKI and JAK2, and is known in the art. Methods for assessing resistance to ruxolitinib are also known in the art.

The term “effective amount” as used herein refers to that amount of an agent that is sufficient to effect treatment, when exposed to a subject. The effective amount will vary depending upon a number of factors, including for example the specific activity of the agent being used, and other characteristics of the subject, including the severity of the disorder, the subject, the age, physical condition, existence of other disease states, nutritional status of the subject and genetic background of the subject. An effective amount can be selected by a person skilled in the art.

Methods for exposing a subject, or cells associated with a disorder, to an immunological agent are known in the art. The exposing may comprise exposure in vivo or exposure ex vivo.

The term “immunological agent” refers to an agent capable of binding specifically, i.e., with antigen specificity, to an antigen. An exemplary immunological agent is an antibody alone, an antibody conjugated to another agent, or an antigen binding part of an antibody.

The term “specific binding” or variants thereof means that an antibody, or an antigen-binding part of fragment thereof, or another construct such as a scFv forms a relatively stable complex with an antigen under physiological conditions. The specific binding may be characterized by an equilibrium dissociation constant of about 1×10⁻⁶ M or smaller (e.g., the smaller the Kd, the tighter the binding). Methods for determining if two molecules bind specifically are well known in the art, for example, equilibrium dialysis, surface plasmon resonance, etc.

In certain embodiments, the immunological agent comprises an antibody and/or antigen binding part thereof. Antibodies and immunological agents comprising an antigen binding part of an antibody are known in the art.

The term “antibody” as used herein refers to an immunoglobulin molecule with the ability to bind an antigenic region of another molecule, and includes monoclonal antibodies, polyclonal antibodies, multivalent antibodies, chimeric antibodies, multispecific antibodies, diabodies and fragments of an immunoglobulin molecule or combinations thereof that have the ability to bind to the antigenic region of another molecule with the desired affinity including a Fab, Fab′, F(ab′)₂, Fv, a single-chain antibody (scFv) or a polypeptide that contains at least a portion of an immunoglobulin (or a variant of an immunoglobulin) that is sufficient to confer specific antigen binding, such as a molecule including one or more CDRs.

In this regard, an immunoglobulin is a tetrameric molecule, each tetramer being composed of two identical pairs of polypeptide chains, each pair having one light chain and one heavy chain. The amino-terminal portion of each chain includes a variable region of about 100 to 110 or more amino acids that is primarily responsible for antigen recognition. The carboxy-terminal portion of each chain defines a constant region primarily responsible for effector function. Human light chains are classified as K and A light chains. Heavy chains are classified as μ, Δ, γ, α, or ε and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. The variable regions of each light/heavy chain pair form the antibody binding site, with the result that an intact immunoglobulin has two binding sites. The variable regions further include hypervariable regions that are directly involved in formation of the antigen binding site. These hypervariable regions are usually referred to as Complementarity Determining Regions (CDR). The intervening segments are referred to as Framework Regions (FR). In both light and heavy chains there are three CDRs (CDR-I to CDR-3) and four FRs (FR-I to FR-4).

In certain embodiments, the antigen-binding part or fragment comprises a Fab, Fab′, F(ab′)₂, Fd, Fv, a single-chain antibody (scFv), a chimeric antibody, a diabody, or a polypeptide that contains at least a portion of an immunoglobulin that is sufficient to confer specific antigen binding.

A Fab fragment is a monovalent fragment consisting of the VL, VH, CL and CH I domains. A F(ab′)₂ fragment is a bivalent fragment including two Fab fragments linked by a disulphide bridge at the hinge region. A Fd fragment consists of the VH and CH I domains. A Fv fragment consists of the VL and VH domains of a single arm of an antibody. A dAb consists of a VH domain. A single chain antibody (scFv) is an antibody in which VL and VH regions are paired to form a monovalent molecule via a synthetic linker that enable them to be made as a single protein chain. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites. In certain embodiments, the immunological agent comprises one or more CDR regions.

Considerations for antibody structure and function are known in the art, for example as described in Chiu et al (2019) Antibodies 8(4): 55, herein incorporated by reference.

Antibody parts or fragments that contain specific binding sites may be generated by a known method. Methods for producing antigen-binding fragments or portions of antibodies are known in the art, for example as described in “Antibody Engineering: Methods and Protocols” (2004) ed. by B.K.C. Lo Humana Press, herein incorporated by reference; and “Antibody Engineering: A Practical Approach” (1996) ed. by J. McCafferty, H.R. Hoogenboom and DJ. Chriswell Oxford University Press, herein incorporated by reference. For example, F(ab′)₂ fragments can be produced by pepsin digestion of the antibody molecule, and Fab fragments can be generated by reducing the disulfide bridges of the F(ab′)₂ fragments. Alternatively, Fab expression libraries may be constructed to allow rapid and easy identification of monoclonal Fab fragments with the desired specificity, as described for example in Huse, W. D. et al. (1989) Science 254:1275-1281, herein incorporated by reference.

Antibodies may be generated using known methods. For the production of antibodies, various hosts including goats, rabbits, rats, mice, humans, and others, may be immunized by injection with an appropriate antigen. Depending on the host species, various adjuvants may be used to increase an immunological response. Such adjuvants include Freund's adjuvant, mineral gels such as aluminium hydroxide, and surface-active substances such as lysolecithin, pluronic polyols, polyanions, peptides, oil emulsions, keyhole limpet hemocyanin, and dinitrophenol. Adjuvants are commercially available.

In certain embodiments, the antibody is a polyclonal antibody. A polyclonal antibody is a mixture of antibodies having different antigen specificities. Methods for producing and isolating polyclonal antibodies are known. In general, polyclonal antibodies are produced from B-lymphocytes. Typically, polyclonal antibodies are obtained directly from an immunized subject, such as an immunized animal.

In certain embodiments, the antibody is a monoclonal antibody. Monoclonal antibodies may be prepared using a technique that provides for the production of antibody molecules by continuous isolated cells in culture. These include, but are not limited to, the hybridoma technique, the human B-cell hybridoma technique, and the EBV-hybridoma technique. Methods for the preparation of monoclonal antibodies include for example Kohler et al. (1975) Nature 256:495-497, herein incorporated by reference: Kozbor et al. (1985) J. Immunol. Methods 81:31-42, herein incorporated by reference: Cote et al. (1983) Proc. Natl. Acad. ScL 80:2026-2030, herein incorporated by reference; and Cole et al. (1984) Mol. Cell Biol. 62:109-120, herein incorporated by reference.

In certain embodiments, the antibody and/or an antigen binding part thereof comprises an isolated antibody or an antigen binding part thereof. Methods for producing and isolating polyclonal and monoclonal antibodies are known.

In certain embodiments, the antibody as described herein has an isotype selected from the group consisting of IgG1, IgG2a, IgG2b, IgG3, IgM and IgA. Determination of the isotype of an antibody may be by a known method.

In certain embodiments, the antibody and/or an antigen binding part thereof is a rat antibody and/or an antigen binding part thereof, a mouse antibody and/or an antigen binding part thereof, a human antibody and/or an antigen binding part thereof, or a humanized antibody and/or an antigen binding part thereof.

Humanized antibodies, or antibodies adapted for non-rejection by other mammals, may be produced by a suitable method known in the art, including for example resurfacing or CDR grafting. In resurfacing technology, molecular modelling, statistical analysis and mutagenesis are combined to adjust the non-CDR surfaces of variable regions to resemble the surfaces of known antibodies of the target host. Strategies and methods for the resurfacing of antibodies, and other methods for reducing immunogenicity of antibodies within a different host are known, for example as described in U.S. Pat. No. 5,639,641. Humanized forms of the antibodies may also be made by CDR grafting, by substituting the complementarity determining regions of, for example, a mouse antibody, into a human framework domain.

Methods for humanizing antibodies are known. For example, the antibody may be generated as described in U.S. Pat. No. 6,180,370, herein incorporated by reference: WO 92/22653, herein incorporated by reference: Wright et al. (1992) Critical Rev. in Immunol. 12 (3,4): 125-168, herein incorporated by reference; and Gu et al. (1997) Thrombosis and Hematocyst 77 (4): 755-759), herein incorporated by reference.

Humanized antibodies typically have constant regions and variable regions other than the complementarity determining regions (CDRs) derived substantially or exclusively from a human antibody and CDRs derived substantially or exclusively from the non-human antibody of interest.

Techniques developed for the production of “chimeric antibodies”, for example the splicing of mouse antibody genes to human antibody genes to obtain a molecule with appropriate antigen specificity and biological activity, may be performed by a suitable method. For example, chimeric antibodies may be produced as described in Morrison, S. L. et al. (1984) Proc. Natl. Acad. ScL 81:6851-6855, herein incorporated by reference: Neuberger, M. S. et al. (1984) Nature 312:604-608, herein incorporated by reference; and Takeda, S. et al. (1985) Nature 314:452-454, herein incorporated by reference.

Immunoassays may be used for screening to identify antibodies and/or antigen binding parts or fragments thereof having the desired specificity. Protocols for competitive binding or immunoradiometric assays using either polyclonal or monoclonal antibodies are known.

Antibody molecules and antigen binding parts or fragments thereof may also be produced recombinantly by methods known in the art, for example by expression in prokaryotic or eukaryotic expression systems. For example, a method for the production of recombinant antibodies is as described in U.S. Pat. No. 4,816,567, herein incorporated by reference. Antigen binding parts or fragments may also be produced by phage display technologies, which are known.

In certain embodiments, the immunological agent comprises a monoclonal antibody, a polyclonal antibody, a multivalent antibody, a chimeric antibody, a multispecific antibody, a diabody, and fragments of an immunoglobulin molecule or combinations thereof that have the ability to bind to the antigenic region such as a Fab, Fab′, F(ab′)₂, Fv, a single-chain antibody (scFv) or a polypeptide that contains at least a portion of an immunoglobulin (or a variant of an immunoglobulin) that is sufficient to confer specific antigen binding, such as a molecule including one or more CDRs.

In certain embodiments, the immunological agent comprises a monoclonal antibody. Methods for exposing a subject, or cells therefrom, to a monoclonal antibody are known in the art. Use of therapeutic antibodies is known in the art. Antibodies (and antigen binding parts thereof) are described herein.

In certain embodiments, the method comprises exposing the subject, and/or cells involved in the myeloproliferative disorder cells, to an immunological agent as described herein.

In certain embodiments, the method comprises exposing the subject, and/or cells involved in the myeloproliferative disorder cells, to an immunological agent comprising an antibody, or an antigen binding part thereof, comprising:
an antibody heavy chain variable region comprising the complementary determining regions of a CDR1 of SEQ ID NO: 18, a CDR2 of SEQ ID NO:22 and a CDR3 of SEQ ID NO:26, and an antibody light chain variable region comprising the complementary determining regions of a CDR1 of SEQ ID NO:27, a CDR2 of SEQ ID NO:30 and a CDR3 of SEQ ID NO:31.

In certain embodiments, the method comprises exposing the subject, and/or cells involved in the myeloproliferative disorder cells, to an immunological agent comprising an antibody, or an antigen binding part thereof, comprising:
an antibody heavy chain variable region comprising the complementary determining regions of a CDR1 of SEQ ID NO:16 or 17, a CDR2 of SEQ ID NO: 19, 20 or 21 and a CDR3 of SEQ ID NO:23, 24 or 25, and an antibody light chain variable region comprising the complementary determining regions of a CDR1 of SEQ ID NO:27, a CDR2 of SEQ ID NO:28 or 29, and a CDR3 of SEQ ID NO:31.

In certain embodiments, the method comprises exposing the subject, and/or cells involved in the myeloproliferative disorder cells, to an immunological agent comprising an antibody, or an antigen binding part thereof, comprising an antibody heavy chain variable region comprising the complementary determining regions of SEQ ID NO: 5 and an antibody light chain variable region comprising complementary determining regions of SEQ ID NO: 7 (4D7).

In certain embodiments, the method comprises exposing the subject, and/or cells involved in the myeloproliferative disorder cells, to an immunological agent comprising an antibody, or an antigen binding part thereof, comprising an antibody heavy chain variable region comprising the complementary determining regions of SEQ ID NO: 9 and an antibody light chain variable region comprising the complementary determining regions of SEQ ID NO: 11 (2D2).

In certain embodiments, the method comprises exposing the subject, and/or cells involved in the myeloproliferative disorder cells, to an immunological agent comprising an antibody, or an antigen binding part thereof, comprising an antibody heavy chain variable region comprising the complementary determining regions of SEQ ID NO: 13 and an antibody light chain variable region comprising the complementary determining regions of SEQ ID NO: 15 (9H11).

Table 1 provides a description of the various sequence identifiers referenced in the present disclosure.

TABLE 1

| Sequence Identifier | Description |
|---|---|
| SEQ ID NO: 1 | Amino acid sequence of |
| | CALR neo-epitope |
| SEQ ID NO: 2 | Amino acid sequence of |
| | polypeptide used for |
| | immunisation |
| SEQ ID NO: 3 | 11 amino acid sequence |
| | of N-terminal peptide |
| SEQ ID NO: 4 | Nucleotide sequence |
| | VH region of 4D7 |
| SEQ ID NO: 5 | Amino acid sequence of |
| | VH region of 4D7 |
| SEQ ID NO: 6 | Nucleotide sequence |
| | VL region of 4D7 |
| SEQ ID NO: 7 | Amino acid sequence of |
| | VL region of 4D7 |
| SEQ ID NO: 8 | Nucleotide sequence |
| | VH region of 2D2 |
| SEQ ID NO: 9 | Amino acid sequence of |
| | VH region of 2D2 |
| SEQ ID NO: 10 | Nucleotide sequence |
| | VL region of 2D2 |
| SEQ ID NO: 11 | Amino acid sequence of |
| | VL region of 2D2 |
| SEQ ID NO: 12 | Nucleotide sequence |
| | VH region of 9H11 |
| SEQ ID NO: 13 | Amino acid sequence of |
| | VH region of 9H11 |
| SEQ ID NO: 14 | Nucleotide sequence |
| | VL region of 9H11 |
| SEQ ID NO: 15 | Amino acid sequence of |
| | VL region of 9H11 |
| SEQ ID NO: 16 | VH CDR1 4D7 |
| SEQ ID NO: 17 | VH CDR1 2D2/9H11 |
| SEQ ID NO: 18 | VH CDR1 Consensus |
| SEQ ID NO: 19 | VH CDR2 4D7 |
| SEQ ID NO: 20 | VH CDR2 2D2 |
| SEQ ID NO: 21 | VH CDR2 9H11 |
| SEQ ID NO: 22 | VH CDR2 Consensus |
| SEQ ID NO: 23 | VH CDR3 4D7 |
| SEQ ID NO: 24 | VH CDR3 2D2 |
| SEQ ID NO: 25 | VH CDR3 9H11 |
| SEQ ID NO: 26 | VH CDR3 Consensus |
| SEQ ID NO: 27 | VL CDR1 |
| | 4D7/2D2/9H11 |
| SEQ ID NO: 28 | VL CDR2 4D7 |
| SEQ ID NO: 29 | VL CDR2 2D2/9H11 |
| SEQ ID NO: 30 | VL CDR2 Consensus |
| SEQ ID NO: 31 | VL CDR3 |
| | 4D7/2D2/9H11 |
| SEQ ID NO: 32 | Primer |
| SEQ ID NO: 33 | Primer |

In certain embodiments, the immunological agent binds to an epitope in the CALR protein comprising SEQ ID NO:1: RRMMRTKMRM RRMRRTRRKM RRKMSPARPR TSCREACLQG WTEA. The location of SEQ ID NO: 1 in the frameshift mutant CALR protein is shown in FIG. 1A. Methods for determining the binding of an immunological agent to an epitope are known in the art.

Epitopes located in the amino acid sequence created by the ins6 and del52 frameshift mutations are also shown in FIG. 1A.

In certain embodiments, the immunological agent binds to an epitope in the N-terminus of SEQ ID NO:1. In certain embodiments, the immunological agent binds to an epitope in the C-terminus of SEQ ID NO:1.

In certain embodiments, the immunological agent binds to an epitope in SEQ ID NO: 3: RRKMSPARRTS

In certain embodiments, the exposing of the subject, or the exposing of cells involved in the myeloproliferative disorder in the subject, comprises administering the immunological agent to a subject.

Methods for administering an agent to a subject are known in the art. In certain embodiments, the agent is administered intravenously. In certain embodiments, the agent is administered via injection, such as by intravenous injection, by intravenous infusion, or subcutaneously. Other administration routes are also contemplated, such as administration orally, nasally, or by administration into the lungs. Methods for formulating antibodies and antigen binding parts thereof for administration are known in the art.

In certain embodiments, the exposing of the cells involved in the myeloproliferative disorder comprises exposing ex vivo cells to the immunological agent. Methods or exposing ex vivo cells to agents are known in the art, as are methods for reintroducing ex vivo cells into a subject for therapeutic purposes.

In certain embodiments, the immunological agent is coupled to a therapeutic or a diagnostic agent. In certain embodiments, the therapeutic agent comprises a toxin, a radioisotope, a chemotherapeutic agent, or a drug. Methods for coupling immunological agents to other agents are known in the art. Methods for utilising agents coupled to immunological agents for therapeutic purposes or diagnostic purposes are known in the art.

In certain embodiments, the immunological agent inhibits the binding of multimers of mutant CALR protein to a thrombopoietin receptor.

In certain embodiments, the immunological agent inhibits the binding of dimers of mutant CALR protein to a thrombopoietin receptor. Methods for assessing the ability of an agent to inhibit binding of multimers/dimers of a protein to a receptor are known in the art. In certain embodiments, the agent inhibits the binding of the protein dimers to the receptor. In certain embodiments, the agent inhibits the formation of dimers or is able to dissociate dimers.

Certain embodiments, the present disclosure provide a method of preventing and/or treating a myeloproliferative disorder in a subject by inhibiting binding of multimers/dimers of mutant CALR protein to the thrombopoietin receptor.

In certain embodiments, the present disclosure provides a method of preventing and/or treating a myeloproliferative disorder in a subject, the disorder associated with the presence of a frameshift mutation in the CALR gene of a cell involved in the myeloproliferative disorder, the method comprising inhibiting binding of dimers of a CALR protein arising from the frameshift mutation in the CALR gene to the thrombopoietin receptor in cells involved with the myeloproliferative disorder.

Methods for assessing binding of dimers of a protein to a receptor are known in the art.

In certain embodiments, the method of inhibition comprises inhibiting the formation of dimers. In certain embodiments, the method of inhibition comprises dissociation of dimers. In certain embodiments, the method of inhibition comprises inhibiting the formation of dimers and the dissociation of dimers.

In certain embodiments, the inhibiting of binding of dimers of a CALR protein comprises inhibiting the binding by use of an agent. Other methods for inhibiting the binding are contemplated.

In certain embodiments, the method comprises exposing the subject, and/or cells involved in the myeloproliferative disorder, to an effective amount of an agent that inhibits binding of the dimers of the CALR protein to the thrombopoietin receptor.

In certain embodiments, the agent comprises an immunological agent. Immunological agents are described herein. In certain embodiments, the immunological agent comprises an antibody or an antigen binding part thereof.

In certain embodiments, the myeloproliferative disorder comprises primary myelofibrosis, thrombocythemia, or secondary acute myeloid leukemia.

Methods for assessing the ability of an agent to inhibit binding of dimers of a protein to a receptor are known in the art. In certain embodiments, the agent inhibits the binding of the protein dimers to the receptor. In certain embodiments, the agent inhibits the formation of dimers or is able to dissociate dimers.

Certain embodiments of the present disclosure provide a method of inhibiting JAK-STAT signalling in a megakaryocyte cell and/or a progenitor cell thereof.

In certain embodiments, the method comprises inhibiting binding of dimers of a mutant CALR to the thrombopoietin receptor, as described herein.

In certain embodiments, the present disclosure provides a method of inhibiting JAK-STAT signalling in a megakaryocyte and/or a progenitor cell thereof, the megakaryocyte and/or the progenitor cell comprising a frameshift mutation in the CALR gene, the method comprising inhibiting binding of dimers of a CALR protein arising from the frameshift mutation in the CALR gene to the thrombopoietin receptor in the megakaryocyte and/or the progenitor cells.

In certain embodiments, the method comprises exposing myelofibroblast cells and/or progenitor cells to an agent that binds to an epitope in the CALR protein arising from the frameshift mutation in the CALR gene, as described herein

In certain embodiments, the present disclosure provides a method of inhibiting JAK-STAT signalling in a megakaryocyte cell and/or a progenitor cell thereof, the megakaryocyte and/or the progenitor cell comprising a frameshift mutation in the CALR gene, the method comprising exposing the myelofibroblast cell and/or the progenitor cell to an effective amount of an immunological agent that binds to an epitope in the CALR protein arising from the frameshift mutation in the CALR gene.

In certain embodiments, the progenitor cell is a megakaryocyte-erythroid progenitor cell.

In certain embodiments, the megakaryocyte cell and/or the progenitor cell are present in vivo in a subject. In this embodiment, the inhibition of JAK-STAT signalling may be used for therapeutic purposes. In certain embodiments, the method is performed in vivo in a subject suffering from, or susceptible to, a primary myelofibrosis, a thrombocythemia, or a secondary acute myeloid leukemia.

In certain embodiments, the megakaryocyte cell and/or the progenitor cell are present ex vivo. In this embodiment, the inhibition of JAK-STAT signalling may also be used for therapeutic purposes.

In certain embodiments, the megakaryocyte cell and/or the progenitor cells are present in vitro. In this embodiment, the inhibition of JAK-STAT signalling may be used, for example, for research purposes.

CALR mutations are described herein. Immunological agents are described herein. Methods for exposing cells to an agent (in vivo, ex vivo or in vitro) are described herein.

Methods for assessing JAK-STAT signalling in vitro and in vivo are known in the art, and examples are described herein.

In certain embodiments, the frameshift mutation in CALR is a +1 frameshift mutation. In certain embodiments, the frameshift mutation comprises a frameshift mutation in exon 9 of the CALR gene. In certain embodiments, the frameshift mutation comprises a CALR^{del52} or a CALR^{ins5} frameshift mutation.

Immunological agents are described herein. In certain embodiments, the immunological agent comprises an antibody and/or an antigen binding part thereof. In certain embodiments, the immunological agent comprises a monoclonal antibody.

In certain embodiments the immunological agent binds to SEQ ID NO: 1. In certain embodiments the immunological agent binds to an epitope located in SEQ ID NO: 1.

In certain embodiments, the immunological agent binds to an epitope in the N-terminus of SEQ ID NO: 1. In certain embodiments, the immunological agent binds to an epitope in the C-terminus of SEQ ID NO:1. In certain embodiments, the immunological agent binds to an epitope in SEQ ID NO:3.

In certain embodiments. the immunological agent is coupled to a therapeutic or diagnostic agent. In certain embodiments, the therapeutic or diagnostic agent is a toxin, a radioisotope, an imaging agent, a drug or a cytotoxic agent.

Certain embodiments of the present disclosure provide a method of inhibiting thrombopoietin-independent proliferation and/or differentiation of a megakaryocyte cell and/or a progenitor cell thereof.

In certain embodiments, the method comprises inhibiting binding of dimers of a mutant CALR to the thrombopoietin receptor, as described herein.

In certain embodiments, the present disclosure provides a method of inhibiting thrombopoietin-independent proliferation and/or differentiation of a megakaryocyte cell and/or a progenitor cell thereof, the megakaryocyte and/or the progenitor cell comprising a frameshift mutation in the CALR gene, the method comprising inhibiting binding of dimers of a CALR protein arising from the frameshift mutation in the CALR gene to the thrombopoietin receptor in the megakaryocyte and/or the progenitor cells.

In certain embodiments, the method comprises exposing myelofibroblast cells and/or the progenitor cells to an agent that binds to an epitope in the CALR protein arising from the frameshift mutation in the CALR gene, as described herein

In certain embodiments, the present disclosure provides a method of inhibiting thrombopoietin-independent proliferation and/or differentiation of a megakaryocyte cell and/or a progenitor cell thereof, the megakaryocyte and/or the progenitor cell comprising a frameshift mutation in the CALR gene, the method comprising exposing the megakaryocyte and/or the progenitor cell to an effective amount of an immunological agent that binds to an epitope in the CALR protein arising from the frameshift mutation in the CALR gene.

In certain embodiments, the megakaryocyte cell and/or the progenitor cell are present in vivo in a subject. In this embodiment, the inhibition of proliferation and/or differentiation may be used for therapeutic purposes. In certain embodiments, the method is performed in vivo in a subject suffering from, or susceptible to, a primary myelofibrosis, a thrombocythemia, or a secondary acute myeloid leukemia.

In certain embodiments, the megakaryocyte cell and/or the progenitor cell are ex vivo cells, for example for introduction into a subject.

In certain embodiments, the megakaryocyte cell and/or the progenitor cell are in vitro cells.

Methods for assessing the proliferation and/or differentiation of megakaryocytes cells and/or progenitors thereof, both in vivo and in vitro, are known on the art.

In certain embodiments, the megakaryocyte cell and/or the progenitor cell are present ex vivo, and the inhibition of proliferation and/or differentiation is used for therapeutic purposes.

In certain embodiments, the megakaryocyte cell and/or the progenitor cells are present in vitro. In this embodiment, the inhibition of proliferation and/or differentiation signalling may be used for example for research purposes.

CALR mutations are described herein. Agents are described herein. Methods for exposing cells to an agent (in vivo, ex vivo or in vitro) are described herein.

In certain embodiments, the frameshift mutation in CALR is a +1 frameshift mutation. In certain embodiments, the frameshift mutation comprises a frameshift mutation in exon 9 of the CALR gene. In certain embodiments, the frameshift mutation comprises a CALR^{del52} or a CALR^{ins5} frameshift mutation.

Immunological agents are described herein. In certain embodiments, the immunological agent comprises an antibody and/or an antigen binding part thereof. In certain embodiments, the immunological agent comprises a monoclonal antibody.

In certain embodiments the immunological agent binds to SEQ ID NO: 1. In certain embodiments the immunological agent binds to an epitope in SEQ ID NO: 1.

In certain embodiments, the immunological agent binds to an epitope in the N-terminus of SEQ ID NO: 1. In certain embodiments, the immunological agent binds to an epitope in the C-terminus of SEQ ID NO:1. In certain embodiments, the immunological agent binds to an epitope in SEQ ID NO:3.

In certain embodiments. the immunological agent is coupled to a therapeutic or diagnostic agent. In certain embodiments, the therapeutic or diagnostic agent is a toxin, a radioisotope, an imaging agent, a drug or a cytotoxic agent.

Certain embodiments of the present disclosure provide an immunological agent.

In certain embodiments, the immunological agent binds to an epitope in the CALR protein arising from a frameshift mutation, as described herein.

In certain embodiments, the present disclosure provides an immunological agent that binds to an epitope in the CALR protein arising from a frameshift mutation.

In certain embodiments, the immunological agent inhibits binding of dimers of a CALR protein arising from a frameshift mutation, as described herein.

In certain embodiments, the present disclosure provides an immunological agent that inhibits binding of dimers of a CALR protein arising from a frameshift mutation to the thrombopoietin receptor.

In certain embodiments, the frameshift mutation is a +1 frameshift mutation. In certain embodiments, the frameshift mutation comprises a frameshift mutation in exon 9 of the CALR gene. In certain embodiments, the frameshift mutation comprises a CALRde^{l52} or a CALR^{ins5} frameshift mutation.

Immunological agents are described herein.

In certain embodiments, the immunological agent comprises an antibody and/or an antigen binding part thereof. In certain embodiments, the immunological agent comprises a monoclonal antibody.

In certain embodiments, the immunological agent comprises a monoclonal antibody, a polyclonal antibody, a multivalent antibody, a chimeric antibody, a multispecific antibody, adiabody and fragments of an immunoglobulin molecule or combinations thereof that have the ability to bind to the antigenic region such as a Fab, Fab′, F(ab′)₂, Fv, a single-chain antibody (scFv) or a polypeptide that contains at least a portion of an immunoglobulin (or a variant of an immunoglobulin) that is sufficient to confer specific antigen binding, such as a molecule including one or more CDRs.

In certain embodiments, the immunological agent inhibits JAK-STAT signalling in megakaryocytes, and/or progenitor cells thereof.

In certain embodiments, the immunological agent inhibits thrombopoietin-independent proliferation and/or differentiation of megakaryocytes and/or progenitor cells.

In certain embodiments, the immunological agent binds to SEQ ID NO: 1.

In certain embodiments, the immunological agent binds to an epitope in the N-terminus of SEQ ID NO:1. In certain embodiments, the immunological agent binds to an epitope in the C-terminus of SEQ ID NO: 1.

In certain embodiments, the immunological agent binds to an epitope in SEQ ID NO: 3.

In certain embodiments, the immunological agent comprises an antibody or an antigen binding part thereof, as described herein.

For example, the immunological agent may comprise all or part of the 4D7, 2D2 or 9H11 antibodies described herein. For example, the CDR sequences of these antibodies are described herein.

In certain embodiments, the immunological agent comprises an antibody, or an antigen binding part thereof, comprising:
an antibody heavy chain variable region comprising the complementary determining regions of a CDR1 of SEQ ID NO: 18, a CDR2 of SEQ ID NO:22 and a CDR3 of SEQ ID NO:26, and
an antibody light chain variable region comprising the complementary determining regions of a CDR1 of SEQ ID NO:27, a CDR2 of SEQ ID NO:30 and a CDR3 of SEQ ID NO:31.

In certain embodiments, the immunological agent comprises an antibody, or an antigen binding part thereof, comprising:
an antibody heavy chain variable region comprising the complementary determining regions of a CDR1 of SEQ ID NO:16 or 17, a CDR2 of SEQ ID NO: 19, 20 or 21 and a CDR3 of SEQ ID NO:23, 24 or 25, and
an antibody light chain variable region comprising the complementary determining regions of a CDR1 of SEQ ID NO:27, a CDR2 of SEQ ID NO:28 or 29, and a CDR3 of SEQ ID NO:31.

In certain embodiments, the immunological agent comprises an antibody, or an antigen binding part thereof, comprising an antibody heavy chain variable region comprising the complementary determining regions of SEQ ID NO: 5 and an antibody light chain variable region comprising complementary determining regions of SEQ ID NO: 7 (that is, as provided for example in 4D7).

In certain embodiments, the immunological agent comprises an antibody, or an antigen binding part thereof, comprising an antibody heavy chain variable region comprising the complementary determining regions of SEQ ID NO: 9 and an antibody light chain variable region comprising the complementary determining regions of SEQ ID NO: 11 (that is, as provided for example in 2D2).

In certain embodiments, the immunological agent comprises an antibody, or an antigen binding part thereof, comprising an antibody heavy chain variable region comprising the complementary determining regions of SEQ ID NO: 13 and an antibody light chain variable region comprising the complementary determining regions of SEQ ID NO: 15 (that is, as provided for example in 9H11).

In certain embodiments. the immunological agent is coupled to a therapeutic or diagnostic agent. In certain embodiments, the therapeutic or diagnostic agent is a toxin, an imaging agent, a radioisotope, a drug or a cytotoxic agent.

Certain embodiments of the present disclosure provide use of an immunological agent, as described herein.

In certain embodiments, the present disclosure provides use of an immunological agent as described herein for one or more of preventing and/or treating a myeloproliferative disorder, inhibiting JAK-STAT signalling, inhibiting thrombopoietin-independent proliferation and/or differentiation, for diagnostic purposes for a disorder as described herein, for prognostic purposes for a disorder as described herein, or for research purposes.

Methods for utilising immunological agents for diagnostic or prognostic purposes are known in the art. Such methods include ELISA based methods or immunohistochemical methods. Other immunological detection methods are contemplated.

Certain embodiments of the present disclosure provide an isolated polypeptide, as described herein.

The term “isolated” refers to a species, such as an antibody, a nucleic acid, a polypeptide, or a cell that has been separated from its natural environment. For example, an isolated nucleic acid or polypeptide may be partially separated from other substances present in the natural environment, or may be in a substantially purified state, being substantially free of other substances with which it is associated in nature or in vivo. In certain embodiments, the species may be synthesized in vitro. Methods for isolating species are known in the art.

In certain embodiments, the present disclosure provides an isolated polypeptide consisting of 10 to 50 amino acids, the polypeptide comprising all or part of an amino acid sequence having at least 90% sequence identity with SEQ ID NO: 1.

In certain embodiments, the isolated polypeptide consists of an amino acid sequence of 40 amino acids or less, 30 amino acids or less, or 20 amino acids or less.

In certain embodiments, the isolated polypeptide consists of 10 to 40 amino acids, 10 to 30 amino acids, 10 to 20 amino acids, 20 to 40 amino acids, or 20 to 30 amino acids.

In certain embodiments, the polypeptide comprises the amino acid sequence of SEQ ID NO:2 and/or SEQ ID NO:3, and/or an amino acid sequence having at least 90% sequence identity therewith.

In certain embodiments, the isolated polypeptide is used to raise antibodies to a neo-epitope of a CALR protein mutant. Methods for using polypeptides as immunogens are known in the art.

Certain embodiments of the present disclosure provide use of a polypeptide as described herein as an immunogen.

Certain embodiments of the present disclosure provide a method of producing an antibody to a polypeptide as described herein. Methods for producing antibodies are as described herein.

Certain embodiments of the present disclosure provide an isolated antibody, or an antigen binding part thereof.

In certain embodiments, the isolated antibody (or antigen binding part thereof) binds to an epitope in the CALR protein arising from a frameshift mutation in the CALR gene.

In certain embodiments, the present disclosure provide an isolated antibody that binds to an epitope in the CALR protein arising from a frameshift mutation in the CALR gene.

In certain embodiments, the isolated antibody (or antigen binding part thereof) inhibits binding of a dimer of a mutant CALR protein to the thrombopoietin receptor.

The antibodies of the present disclosure have therapeutic uses, prognostic and/or diagnostic uses, and use as research reagents, as described herein.

Methods for producing antibodies (or an antigen binding part thereof) are known in the art, and are described herein.

In certain embodiments, the frameshift mutation in CALR is a +1 frameshift mutation. In certain embodiments, the frameshift mutation comprises a frameshift mutation in exon 9 of the CALR gene. In certain embodiments, the frameshift mutation comprises a CALR^{del52} or a CALR^{ins5} frameshift mutation.

In certain embodiments the antibody (or an antigen binding part thereof) binds to SEQ ID NO: 1.

In certain embodiments, the antibody (or an antigen binding part thereof) binds to an epitope in the N-terminus of SEQ ID NO:1. In certain embodiments, the antibody binds to an epitope in the C-terminus of SEQ ID NO: 1. In certain embodiments, the binds to an epitope in SEQ ID NO:3.

Examples of antibodies include antibodies 4D7, 2D2 or 9H11 as described herein. Further characterisation of these antibodies is described herein.

Certain embodiments of the present disclosure provide a hybridoma expressing an antibody as described herein. Methods for producing hybridomas are known in the art.

Certain embodiments of the present disclosure provide a method of producing an antibody, as described herein.

In certain embodiments, the present disclosure provides a method of producing an antibody to a frameshift mutant of the CALR protein, the method comprising raising an antibody to a polypeptide comprising an amino acid sequence from all or part of a region of the calreticulin protein arising from a frameshift mutation in the CALR gene.

In certain embodiments, the present disclosure provides a method of producing an antibody to a frameshift mutant of the CALR protein, the method comprising raising an antibody to a polypeptide comprising a neo-epitope arising from the frameshift mutation in the CALR gene.

In certain embodiments, the frameshift mutation in CALR is a +1 frameshift mutation. In certain embodiments, the frameshift mutation comprises a frameshift mutation in exon 9 of the CALR gene. In certain embodiments, the frameshift mutation comprises a CALR^{del52} or a CALR^{ins5} frameshift mutation.

Certain embodiments of the present disclosure provide an antibody (or antigen binding part thereof) comprising one of more CDR regions identified in one or more of antibodies 4D7, 2D2 and/or 9H11, as described herein.

The term “CDR” or “complementarity-determining region” refers to the amino acid sequence of the hypervariable region of a heavy chain and a light chain of an immunoglobulin (Kabat et al., Sequences of Proteins of Immunological Interest, 4th ed., U.S. Department of Health and Human Services, National Institutes of Health (1987)). Each of the heavy chain (CDRH1, CDRH2 and CDRH3) and the light chain (CDRL1, CDRL2 and CDRL3) includes three CDRs. The CDR provides major contact residue for binding of an antibody to an antigen or an epitope.

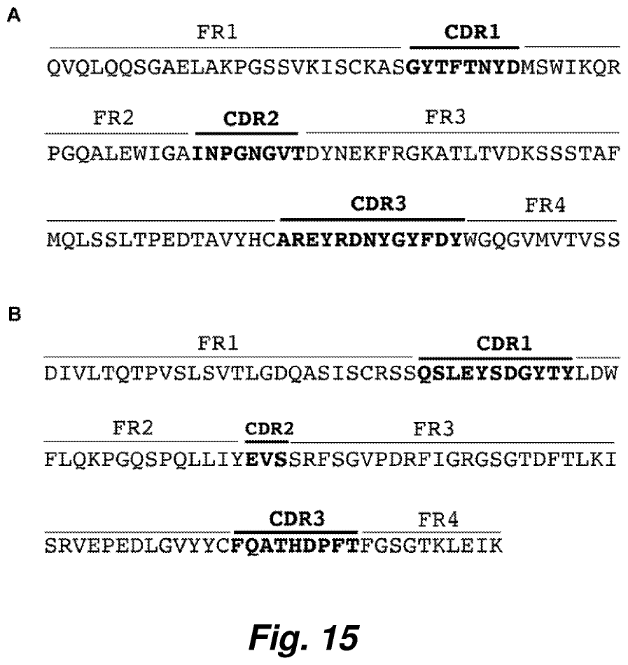

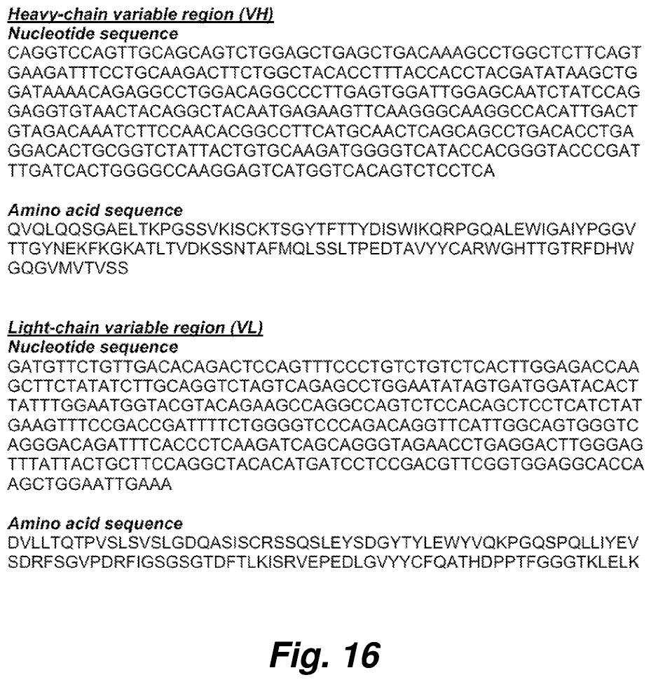

Based on the sequence of the 4D7, 2D2 and 9H antibodies identified herein, the following consensus sequences (see FIGS. 15 and 16) were determined:

V_{H} Region
(i) CDR1 Sequences
Consensus
(SEQ ID NO: 18)
GYTTT(T/N)YD
(ii) CDR2 Sequences
Consensus:
(SEQ ID NO: 22)
I(Y/N)PG(G/N)(G/V/E)(T/V)T
(iii) CDR3 Sequences
Consensus:
(SEQ ID NO: 26)
AR(W/E)(G/Y)(H/R)(T/D)(T/N)(G/Y)(G/T)(R/Y)FD(H/Y)
V_{L} Region
(i) CDR1 Sequences
Consensus:
(SEQ ID NO: 27)
QSLEYSDGYTY
(ii) CDR2 Sequences
Consensus:
(SEQ ID NO: 30)
EVS(D/S)RFSG
(iii) CDR3 Sequences
Consensus:
(SEQ ID NO: 31)
FQATHDPFT

In certain embodiments, the present disclosure provides an antibody or an antigen binding part thereof comprising:
an antibody heavy chain variable region comprising the complementary determining regions of a CDR1 of SEQ ID NO: 18, a CDR2 of SEQ ID NO:22 and a CDR3 of SEQ ID NO:26, and
an antibody light chain variable region comprising the complementary determining regions of a CDR1 of SEQ ID NO:27, a CDR2 of SEQ ID NO:30 and a CDR3 of SEQ ID NO:31;
or a functional variant having at least 90% sequence identity to any one or more of the aforementioned complementary determining regions.

In certain embodiments, the present disclosure provides an antibody or an antigen binding part thereof comprising:
an antibody heavy chain variable region comprising the complementary determining regions of a CDR1 of SEQ ID NO:16 or 17, a CDR2 of SEQ ID NO: 19, 20 or 21 and a CDR3 of SEQ ID NO:23, 24 or 25, and
an antibody light chain variable region comprising the complementary determining regions of a CDR1 of SEQ ID NO:27, a CDR2 of SEQ ID NO:28 or 29, and a CDR3 of SEQ ID NO:31,
or a functional variant having at least 90% sequence identity to any one or more of the aforementioned complementary determining regions.

In certain embodiments, the antibody and/or antigen binding part thereof comprises a functional variant of an antibody, or an antigen binding part thereof, as described herein with at least 90%, at least 91%, at least 92%, at least 93%, at least at least 94%, at least 95%, at least 96%, at least 97%, at least 98% or at least 99% sequence identity, greater than 90%, greater than 91%, greater than 92%, greater than 93%, greater than 94%, greater than 95%, greater than 96%, greater than 97%, greater than 98% or at greater than 99% sequence identity to the above-described CDRs. Methods for determining sequence identity are known in the art, for example the NCBI BLAST suite of programs.

In certain embodiments, the antibody and/or the antigen binding part thereof are humanised.

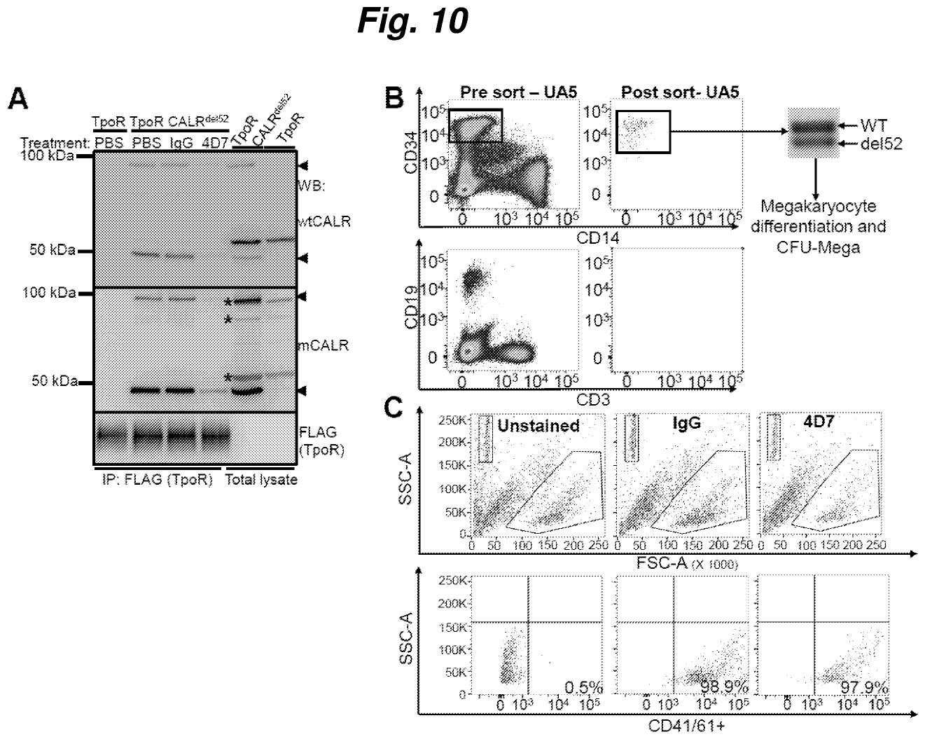

In certain embodiments, the present disclosure provides an antibody with the VH and VL complementarity determining regions of antibody 4D7 as described herein. The CDRs of 4D7 are shown in FIG. 10.

In certain embodiments, the present disclosure provides an antibody or an antigen binding part thereof comprising an antibody heavy chain variable region comprising the complementary determining regions of SEQ ID NO: 5 and an antibody light chain variable region comprising complementary determining regions of SEQ ID NO: 7 (4D7).

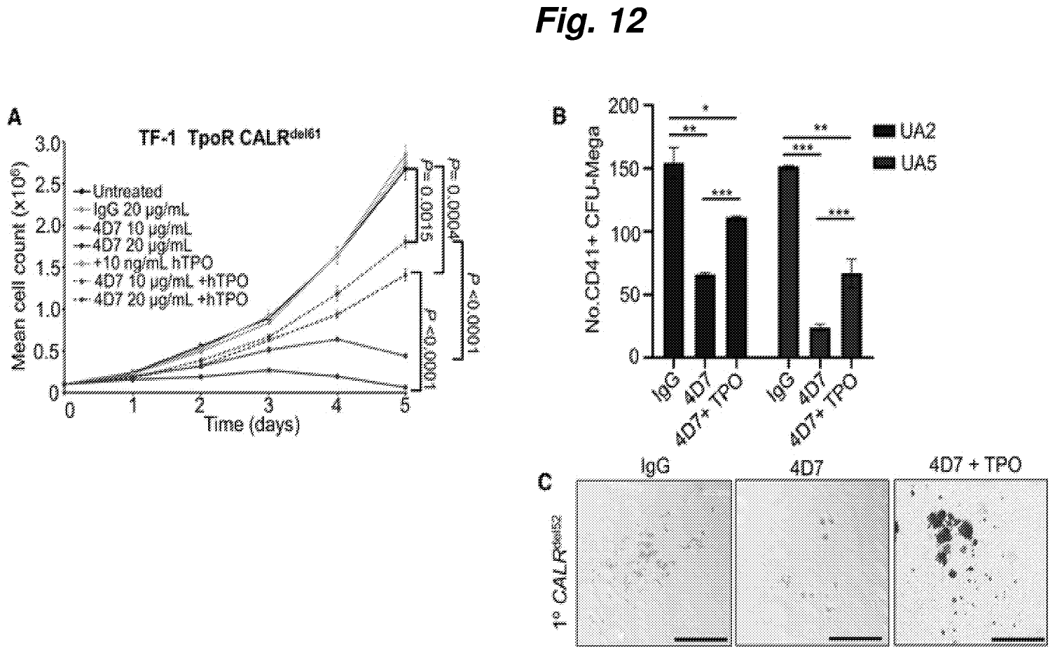

In certain embodiments, the present disclosure provides an antibody with the VH and VL complementarity determining regions of antibody 2D2 as described herein. The CDRs of 2D2 are shown in FIG. 12.

In certain embodiments, the present disclosure provides an antibody or an antigen binding part thereof comprising: an antibody heavy chain variable region comprising the complementary determining regions of SEQ ID NO: 9 and an antibody light chain variable region comprising the complementary determining regions of SEQ ID NO: 11 (2D2).

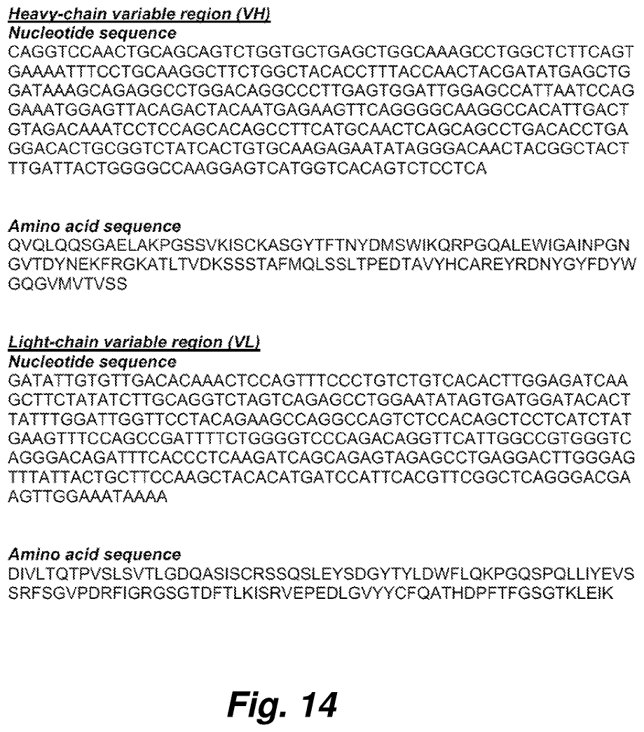

In certain embodiments, the present disclosure provides an antibody with the VH and VL complementarity determining regions of antibody 9H11 described herein. The CDRs of 9H11 are shown in FIG. 14.

In certain embodiments, the present disclosure provides an antibody or an antigen binding part thereof comprising an antibody heavy chain variable region comprising the complementary determining regions of SEQ ID NO: 13 and an antibody light chain variable region comprising the complementary determining regions of SEQ ID NO: 15 (9H11).

In certain embodiments, an antibody or an antigen binding part thereof are humanized. Methods for humanization of antibodies are known in the art and described herein.

Certain embodiments of the present disclosure provide a hybridoma expressing an antibody as described herein.

Certain embodiments of the present disclosure provide a chimeric antigen receptor.

In certain embodiments, the present disclosure provides a method of preventing and/or treating a myeloproliferative disorder using T cells engineered to express a chimeric receptor antigen as described herein. Methods for producing chimeric antigen receptors based on antibody sequences are known in the art. Methods for creating chimeric antigen expression cells, such as CAR-T cells, are known in the art.

In certain embodiments, the present disclosure provides method of preventing and/or treating a myeloproliferative disorder in a subject, the myeloproliferative disorder associated with the presence of a frameshift mutation in the CALR gene of a cell involved in the myeloproliferative disorder, the method comprising exposing the subject, and/or cells involved in the myeloproliferative disorder, to T cells expressing a chimeric antigen receptor as described herein.

In certain embodiments, the present disclosure provides a chimeric antigen receptor comprising an extracellular domain having a domain that binds to an epitope in a CALR protein arising from a frameshift mutation in the CALR gene.

In certain embodiments, the present disclosure provides a chimeric antigen receptor comprising an extracellular domain having a binding domain that inhibits binding of a dimer of a frameshift mutant CALR protein to the thrombopoietin receptor.

The term “chimeric antigen receptor (CAR)” refers to an artificially constructed hybrid protein (fusion protein) or polypeptide containing a target-binding domain (e.g. single-chain variable fragment (scFv)) linked to an effector cell-signalling or effector cell-activating domain (e.g. T-cell signalling or T-cell activating domain). In general, the chimeric antigen receptor has the ability of redirecting T-cell specificity and reactivity toward a selected target in a non-MHC restricted manner by taking advantage of the antigen-binding property of a monoclonal antibody. The non-MHC-restricted antigen recognition confers the ability to recognize an antigen on T-cells expressing CAR, thus bypassing the major mechanism of tumor escape. Moreover, when expressed in T-cells, the CAR advantageously does not dimerize with the endogenous T-cell receptor (TCR) alpha and beta chains.

Methods for constructing chimeric antigen receptors are known in the art, for example as described in Guedan et al. (2019) Molecular Therapy: Methods & Clinical development 12:145-157, herein incorporated by reference.

The chimeric antigen receptor of the present disclosure includes a transmembrane domain because it is expressed on the cell surface. The transmembrane domain may be a transmembrane domain of a protein selected from a group consisting of the T-cell receptor alpha, beta or zeta chain, CD28, CD3 epsilon, CD45, CD4, CD5, CD8, CD9, CD16, CD22, CD33, CD37, CD64, CD80, CD86, CD134, CD137 and CD154, although other transmembrane domains are contemplated.

The costimulatory domain of the chimeric antigen receptor of the present disclosure may be a functional signalling domain obtained from a protein selected from a group consisting of MHC class I molecule, TNF receptor protein, immunoglobulin-like protein, cytokine receptor, integrin, signalling lymphocytic activation molecule (SLAM), activating NK cell receptor, BTLA (B-and T-lymphocyte attenuator), Toll-like ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRFI), NKp44, NKp30, NKp46, CD19, CD4, CD8alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGBI, CD29, ITGB2, CD18, LFA-1, ITGB7, NKG2D, NKG2C, TNFR2, TRANCE/RANKL, DNAMI (CD226), SLAMF4 (CD244, 2B4), CD84, CD96 (Tactile), CEACAMI, CRTAM, Ly9 (CD229), CD160 (BY55), PSGLI, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand binding specifically to CD83, although other co-stimulatory domains are contemplated.

The CALR-binding domain of the chimeric antigen receptor of the present disclosure is typically linked to a transmembrane domain by a hinge domain, such as the IgG4 hinge, CD8 hinge or IgD hinge.

In certain embodiments, the present disclosure provides a chimeric antigen receptor comprising an extracellular domain having a binding domain that binds to an epitope (neo-epitope) in a CALR protein arising from a frameshift mutation in the CALR gene.

In certain embodiments, the present disclosure provides a chimeric antigen receptor comprising an extracellular domain having a binding domain that binds to a neo-epitope in a CALR protein arising from a frameshift mutation in the CALR gene.

In certain embodiments, the frameshift mutation is a +1 frameshift mutation. In certain embodiments, the frameshift mutation comprises a frameshift mutation in exon 9 of the CALR gene. In certain embodiments, the frameshift mutation comprises a CALRde^{l52} or a CALR^{ins5} frameshift mutation.

In certain embodiments, the present disclosure provides a chimeric antigen receptor comprising an extracellular domain comprising a binding domain that binds to SEQ ID NO:1.

In certain embodiments, the present disclosure provides a chimeric antigen receptor comprising an extracellular domain comprising a binding domain that binds to SEQ ID NO:1: a transmembrane domain; and an intracellular signalling domain that activates a cellular function. In certain embodiments, the binding domain binds to a N-terminal region or a C-terminal region of SEQ ID NO:1.

In certain embodiments, the present disclosure provides a chimeric antigen receptor comprising an extracellular domain comprising a binding domain from a heavy chain variable region and a light chain variable region of an antibody as described herein.

In certain embodiments, the present disclosure provides a chimeric antigen receptor comprising an extracellular domain comprising a binding domain comprising:
an antibody heavy chain variable region comprising the complementary determining regions of a CDR1 of SEQ ID NO: 18, a CDR2 of SEQ ID NO:22 and a CDR3 of SEQ ID NO:26, and
an antibody light chain variable region comprising the complementary determining regions of a CDR1 of SEQ ID NO:27, a CDR2 of SEQ ID NO:30 and a CDR3 of SEQ ID NO:31:
or a functional variant having at least 90% sequence identity to any one or more of the aforementioned complementary determining regions

In certain embodiments, the present disclosure provides a chimeric receptor antigen comprising an extracellular domain comprising a binding domain comprising:
an antibody heavy chain variable region comprising the complementary determining regions of a CDR1 of SEQ ID NO:16 or 17, a CDR2 of SEQ ID NO: 19, 20 or 21, and a CDR3 of SEQ ID NO:23, 24 or 25, and
an antibody light chain variable region comprising the complementary determining regions of a CDR1 of SEQ ID NO:27, a CDR2 of SEQ ID NO:28 or 29, and a CDR3 of SEQ ID NO:31.

In certain embodiments, the chimeric antigen receptor comprises a CDR as described above with at least 91%, 92%, 93%, 94%, 95%, 96%, 97%, 98% or 99% sequence identity to one or more of the above-described CDRs, embodiments, the chimeric antigen receptor comprises a CDR as described above with greater than 91%, greater than 92%, greater than 93%, greater than 94%, greater than 95%, greater than 96%, greater than 97%, greater than 98% or greater 99% sequence identity to one or more of the above-described CDRs.

In certain embodiments, the present disclosure provides a chimeric antigen receptor with the VH and VL complementarity determining regions of antibody 4D7 as described herein. The CDRs of 4D7 are shown in FIG. 10.

In certain embodiments, the present disclosure provides a chimeric antigen receptor comprising an antibody heavy chain variable region comprising the complementary determining regions of SEQ ID NO: 5 and an antibody light chain variable region comprising the complementary determining regions of SEQ ID NO: 7.

In certain embodiments, the present disclosure provides a chimeric antigen receptor comprising an antibody with the VH and VL complementarity determining regions of antibody 2D2 as described herein. The CDRs of 2D2 are shown in FIG. 12.

In certain embodiments, the present disclosure provides a chimeric antigen receptor comprising an antibody heavy chain variable region comprising the complementary determining regions of SEQ ID NO: 9, and an antibody light chain variable region comprising the complementary determining regions of SEQ ID NO: 11.

In certain embodiments, the present disclosure provides a chimeric receptor antigen with the VH and VL complementarity determining regions of antibody 9H11 described herein. The CDRs of 9H11 are shown in FIG. 14.

In certain embodiments, the present disclosure provides a chimeric antigen receptor comprising an antibody heavy chain variable region comprising the complementary determining regions of SEQ ID NO: 13 and an antibody light chain variable region comprising the complementary determining regions of SEQ ID NO: 15.

Certain embodiments of the present disclosure provide a cell expressing a chimeric antigen receptor as described herein. In certain embodiments, the cellar is used to prevent or treat a myeloproliferative disorder.

In certain embodiments, the cell is a T cell.

Certain embodiments of the present disclosure provide an isolated nucleic acid.

In certain embodiments, the present disclosure provide a nucleic acid molecule comprising a nucleotide sequence encoding a polypeptide as described herein, an antibody (or an antigen binding part thereof) as described herein, or a chimeric antigen receptor as described herein.

The term “nucleic acid” as used herein refers to an oligonucleotide or a polynucleotide and includes for example DNA, RNA, DNA/RNA, a variant or DNA and/or RNA (for example a variant of the sugar-phosphate backbone and/or a variant of one or more bases, such as methylation), and may be single stranded, double stranded, non-methylated, methylated or other forms thereof. In certain embodiments, the nucleic acid is a non-naturally occurring nucleic acid, a naturally occurring nucleic acid, a nucleic acid of genomic origin, a mitochondrial nucleic acid, a nucleic acid of cDNA origin (derived from a mRNA), a nucleic acid derived from a virus, a nucleic acid of synthetic origin, a single stranded DNA, a double stranded DNA, an analogue of DNA and/or RNA, and/or a derivative, fragment and/or combination of any of the aforementioned. Examples of derivatives also include nucleic acids that have a blocking group at the 5′ and/or 3′ ends for example to improve stability, and/or nucleic acids fused to other molecules. Other types of nucleic acids are contemplated. Methods for producing nucleic acids are known and include for example nucleic acids produced by recombinant DNA technology or nucleic acids produced by chemical synthesis.

The term “nucleic acid” as used herein also refers to a specified nucleic acid, or a nucleic acid comprising a nucleotide sequence which is the complement of the nucleic acid, a nucleic acid comprising a nucleotide sequence with greater than 70%, 75%, 80%, 85%, 90%, 95% or 99% sequence identity to the specified nucleic acid, or a nucleic acid comprising a nucleotide sequence with greater than 70%, 75%, 80%, 85%, 90%, 95, or 99% sequence identity to the complement of the specified nucleic acid. Other levels of sequence identity are contemplated.

Methods for using recombinant DNA technology to produce, manipulate or utilise nucleic acids are known in the art.

Certain embodiments of the present disclosure provide a vector comprising a nucleic acid as described herein.

The term “vector” refers to a composition of a material which contains an isolated nucleic acid and can be used to deliver the isolated nucleic acid into a cell or to a vector which is used for the expression of a specific nucleotide sequence.

Typically, an expression vector comprises an expression control sequence operably linked to a nucleotide sequence to be expressed for expression of a target gene in a host cell. The expression vector includes a cis-acting element sufficient for expression and other elements for expression can be provided by a host cell or an in vitro expression system. The expression vector includes a plasmid vector including a recombinant polynucleotide: a cosmid vector; and a viral vector such as a bacteriophage vector, an adenoviral vector, a lentiviral vector, a retroviral vector and an adeno-associated viral vector.

The recombinant vector system of the present disclosure may be constructed according to various methods known in the art. Specific methods are described in Sambrook et al., Molecular Cloning, A Laboratory Manual, Cold Spring Harbor Laboratory Press (2001), which is incorporated into the present disclosure by reference.

The vector of the present disclosure may be constructed as a vector for gene cloning, a vector for protein expression, or a vector for gene delivery. In addition, the vector of the present disclosure may be constructed by using a prokaryotic cell or a eukaryotic cell as a host cell.

For example, when the vector of the present disclosure is an expression vector and a eukaryotic cell is used as a host cell, a promoter derived from the genome of a mammalian cell (e.g., metallothionein promoter, B-actin promoter, human hemoglobin promoter and human muscle creatine promoter) or a promoter derived from a mammalian virus (e.g., adenovirus late promoter, vaccinia virus 7.5K promoter, SV40 promoter, cytomegalovirus promoter, tk promoter of HSV, mouse mammary tumor virus (MMTV) promoter, LTR promoter of HIV, Moloney virus promoter, Epstein-Barr virus (EBV) promoter and Rous sarcoma virus (RSV) promoter) may be used, and they generally have a polyadenylation sequence as a transcription termination sequence.

Certain embodiments of the present disclosure provide a cell comprising a polypeptide as described herein, an antibody or an antigen binding part thereof as described herein, a chimeric receptor antigen as described herein a nucleic acid as described herein, or a vector as described herein.

In certain embodiments, the cell is a prokaryotic cell.

In certain embodiments, the cell is a eukaryotic cell, such as a mammalian cell.

Examples of cells include a dendritic cell, a killer dendritic cell, a mast cell, a natural killer cell, a B lymphocyte, a T lymphocyte, a macrophage and precursor cells thereof. The T lymphocyte cell may be selected from a group consisting of an inflammatory T lymphocyte, a cytotoxic T lymphocyte, a regulatory T lymphocyte or a helper T lymphocyte.

In certain embodiments, the cell is a T-cell, a Natural Killer cell, or a Natural Killer T cell. Other types of cells are contemplated.

Certain embodiments of the present disclosure provide a method of killing a target cell expressing a mutant CALR protein arising from a frameshift mutation in the CALR gene.

In certain embodiments, the present disclosure provides a method of killing a target cell expressing a mutant CALR protein arising from a frameshift mutation in the CALR gene, the method comprising exposing the target cell to a cell expressing a chimeric antigen receptor as described herein.

In certain embodiments, the target cell comprises a megakaryocyte and/or a megakaryocyte-erythroid progenitor cell.

In certain embodiments, the target cell is present in vivo. In certain embodiments, the target cell is present in a subject. In certain embodiments, the target cell is present in a subject suffering from, or susceptible to, a myeloproliferative disorder.

In certain embodiments, the myeloproliferative disorder comprises primary myelofibrosis, thrombocythemia, or secondary acute myeloid leukemia.

Method for using cells expressing a chimeric antigen receptor for use in a subject for therapeutic uses are known in the art.

Certain embodiments of the present disclosure provide use of a chimeric antigen receptor, or cells expressing a chimeric receptor antigen, as described herein for preventing and/or treating a myeloproliferative disorder.

Certain embodiments of the present disclosure provide a method of preventing or treating a CALR mutant myeloproliferative disorder in a subject, the method exposing the subject to a cell expressing a chimeric receptor antigen as described herein.

Certain embodiments of the present disclosure provide a pharmaceutical composition.

In certain embodiments, the present disclosure provides a pharmaceutical composition comprising an antibody (or an antigen binding part thereof) as described herein, a polypeptide as described herein, a nucleic acid as described herein, a vector as described herein, or a cell as described herein.

The formulation of pharmaceutical compositions for specific purposes is known in the art. The pharmaceutical compositions will typically also include a pharmaceutically acceptable carrier and may include additional numerous various excipients, dosage forms, dispersing agents and the like that are suitable for use in connection with the administration of an agent as described herein and/or the formulation into medicaments or pharmaceutical compositions.

Formulations are known and described in, for example, Remington's Pharmaceutical Sciences, 17th ed., Mack Publishing Company, Easton, Pa., 1985, which is incorporated herein by reference in its entirety.

For the administration of an antibody and/or an antigen binding part thereof, these may be incorporated into a pharmaceutical composition, generally along with a pharmaceutically acceptable carrier, suitable for administration to a subject in vivo.

For the administration of chimeric antigen receptor, these may be provided in a pharmaceutical composition comprising a cell expressing the chimeric receptor antigen, along with a pharmaceutically acceptable carrier, or a nucleic acid comprising a nucleotide sequence expressing a chimeric receptor antigen.

Certain embodiments of the present disclosure provide a method for preventing and/or treating a subject for a CALR mutant myeloproliferative disorder by assessing the subject for the presence of a frameshift mutant in the CALR gene.

In certain embodiments, the present disclosure provides a method of preventing or treating a subject for a CALR mutant myeloproliferative disorder, the method comprising:
assessing the subject for the presence of a frameshift mutant in the CALR gene in cells involved in the myeloproliferative disorder; and
treating the subject identified to have the frameshift mutation in the CALR gene with an effective amount of an immunological agent that binds to an epitope in the CALR protein arising from the frameshift mutation in the CALR gene.

In certain embodiments, the CALR mutant myeloproliferative disorder is primary myelofibrosis, thrombocythemia, or secondary acute myeloid leukemia.

Immunological agents are described herein.

In certain embodiments, the immunological agent is an antibody and/or an antigen binding part thereof, as described herein.

In certain embodiments, the present disclosure provides a method of preventing or treating a subject for a CALR mutant myeloproliferative disorder, the method comprising:
assessing the subject for the presence of a frameshift mutant in the CALR gene in cells involved in the myeloproliferative disorder; and
treating the subject identified to have the frameshift mutation in the CALR gene with an effective amount of a cell expressing a chimeric receptor antigen as described herein.

Methods for treatment are as described herein.

Certain embodiments of the present disclosure provide a kit for performing a method as described herein.

Typically, kits will contain one or more reagents to assist with performing the method, and may also contain negative or positive controls, and instructions.

In certain embodiments, the present disclosure provides use of an antibody (and/or antigen binding part thereof) as described herein in a kit for diagnosis, prognosis or research purposes.

Such kits may be prepared so as to be suitable for various immunoassay or immunostaining applications. The immunoassay or immunostaining includes radioimmunoassay, radioimmunoprecipitation, immunoprecipitation, enzyme-linked immunosorbent assay (ELISA), capture ELISA, inhibition or competition assay, sandwich assay, flow cytometry, immunofluorescence staining and immunoaffinity purification, although not being limited thereto. Methods for the immunoassay or immunostaining are known in the art, for example as described in Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, N J, 1984; and Ed Harlow and David Lane, Using Antibodies: A Laboratory Manual, Cold Spring Harbor Laboratory Press, 1999.

Methods for producing chimeric antigen receptors based on determination of the structure of antibodies are known in the art, for example as described in “Chimeric Antigen Receptor T Cells, Development and Production” (2020) Editors: Kamilla Swiech, Kelen Cristina Ribeiro Malmegrim, Virginia Picanço-Castro. Methods on Molecular Biology 2086. Humana Press, which is herein incorporated by reference.

Standard techniques and equipment may be used for recombinant DNA technology, oligonucleotide synthesis, molecular biology, cell biology, immunological technology, and enzymatic reactions. The foregoing techniques and procedures may be generally performed according to methods known in the art and/or as commercially available, and are as described for example in Sambrook et al. Molecular Cloning: A Laboratory Manual (4th ed., Cold Spring Harbor Laboratory Press, Cold Spring Harbor, N.Y. (1989)) and Ausubel et al Current Protocols in Molecular Biology (2003) John Wiley & Sons, both of which are herein incorporated by reference.

The present disclosure is further described by the following examples. It is to be understood that the following description is for the purpose of describing particular embodiments only and is not intended to be limiting with respect to the above description.

### Example 1—Selective Targeting of Human Calreticulin Mutated Myelofibrosis Progenitor Cells with a Neoepitope-Directed Monoclonal Antibody

### Abstract

Calreticulin (CALR) is recurrently mutated in myelofibrosis via a frameshift that removes an endoplasmic reticulum retention signal, creating a neoepitope potentially targetable by immunotherapeutic approaches. We developed a specific rat monoclonal IgG2a antibody, 4D7, directed against the common sequence encoded by both insertion and deletion mutations. 4D7 selectively bound to cells co-expressing mutant CALR and thrombopoietin receptor (TpoR) and blocked JAK-STAT signalling, TPO-independent proliferation and megakaryocyte differentiation of mutant CALR myelofibrosis progenitors by disrupting the binding of CALR dimers to TpoR. Importantly, 4D7 inhibited proliferation of patient samples with both insertion and deletion CALR mutations but not JAK2 V617F (55% decreased CD41+ cells, P<0.001: 46% decreased megakaryocyte colonies, P<0.001) and prolonged survival in xenografted bone marrow models of mutant CALR-dependent myeloproliferation (log-rank hazard ratio 0.24, P=0.003). Together, our data demonstrate a novel therapeutic approach to target a problematic disease driven by a recurrent somatic mutation that would normally be considered undruggable.

### INTRODUCTION

Primary myelofibrosis (PMF) is the most severe Philadelphia-negative myeloproliferative neoplasm, and is characterised by marrow fibrosis and chronic inflammatory symptoms with a 5-year survival of less than 50% (Baade et al, 2019). Although rare, PMF affects both young and older adults and can evolve into acute leukemia in >15% of cases (Passamonti et al, 2012). Mutations within the gene encoding calreticulin (CALR) are the second most common genetic aberration associated with PMF, observed in 70% of non-JAK2^{V617F} cases (Klampfl et al, 2013: Nangalia et al, 2013). Importantly, myelofibrosis patients with CALR mutations do not effectively respond to JAK inhibitor therapy and no CALR-specific therapy has been developed (Ross et al, 2021).

Virtually all CALR mutations identified in PMF are small insertions or deletions clustered within exon 9. The two most common mutations identified include a 52 bp deletion (type 1) or a 5 bp insertion (type 2) (Klampfl et al., 2013: Nangalia et al., 2013). These frameshift mutations lead to a neoepitope peptide sequence which is thought to directly or indirectly activate the thrombopoietin receptor (TpoR) by a poorly defined mechanism that is dependent on glycan binding sites, N-terminal chaperone domain and the novel C-terminal tail of the mutant protein (Araki et al, 2016; Chachoua et al, 2016; Elf et al, 2016; Marty et al, 2016). All somatic CALR mutations seen in PMF result in a +1 frameshift, leading to a common altered peptide sequence with loss of the negative charged C-terminal calcium-binding domain, gain of a lysine/arginine rich segment followed by a stop codon and loss of the KDEL sequence that constitutes an endoplasmic reticulum retention signal (Klampfl et al., 2013: Nangalia et al., 2013). Recently, it has been demonstrated that without this localisation signal, mutant CALR protein is passively secreted from cells, and is detectable in cultured cell supernatants (Han et al, 2016; Liu et al, 2020: Masubuchi et al, 2020) but whether TpoR activation occurs before or after cell surface exposure and whether or not it is accessible to an extracellularly-acting therapeutic is not clear (How et al, 2019). Recent data suggests that multimerization of mutant CALR monomers is absolutely required for mutant CALR TPO-independent proliferation (Araki et al, 2019). There are subtle differences in prognosis and biochemistry between type 1 and type 2 CALR mutations (How et al., 2019), which are classified by the extent of elimination of negatively charged residues in the mutant protein compared to wild type. Ideally, a therapeutic would have activity against both type 1 and type 2 CALR mutations with minimal to no effect on normal hematopoeisis.

Here, we demonstrate a novel therapeutic strategy for PMF by developing a monoclonal antibody with specificity for the unstructured mutant CALR peptide that inhibits TpoR activation through a distinct mechanism. Our data establish that CALR mutants form disulfide-linked homodimers and that these bring together their constitutively associated TpoR to cause receptor activation. Treatment of mutant CALR cells with the 4D7 monoclonal antibody inhibits TpoR activation, constitutive STAT and ERK phosphorylation, TPO-independent megakaryocyte differentiation from patients with both type 1 and type 2 frameshift mutations, and prolong survival in xenograft models of mutant CALR-driven myeloproliferation. Together, our data demonstrates a novel approach to target frameshift mutations in cancer that were previously considered undruggable.

### Results and Discussion

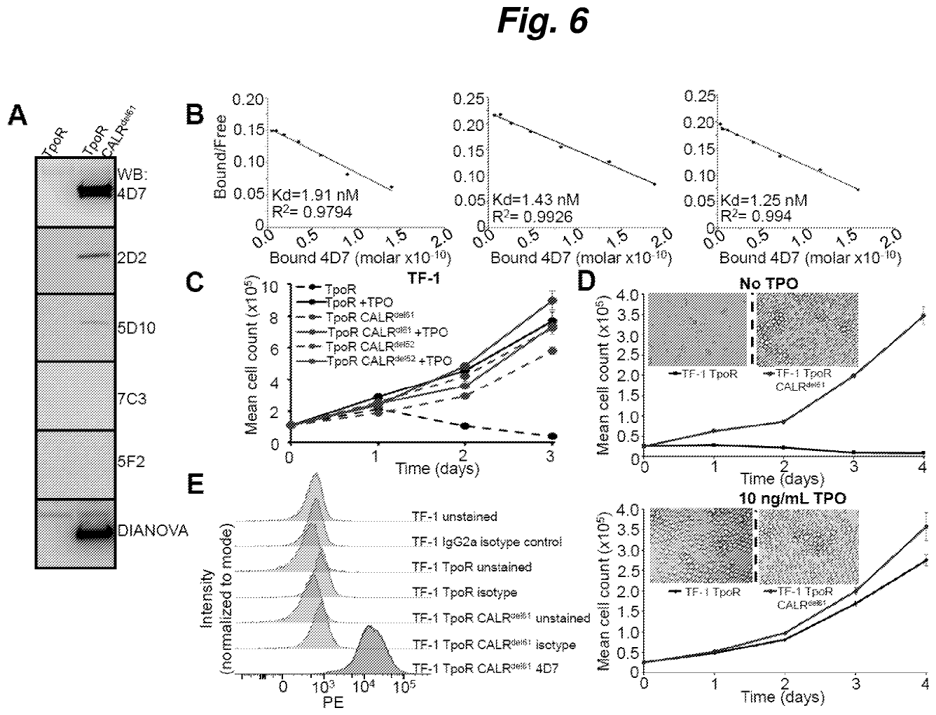

Both type 1 and type 2 CALR mutations in myelofibrosis result in a loss of the endoplasmic reticulum retention signal KDEL producing aberrant localization of mutant protein (How et al., 2019) and the acquisition of a 31 amino acid-long sequence towards the C-terminus (FIG. 1A). We reasoned that this neoepitope would be an ideal target for precision medicine approaches and set out to develop a panel of rat monoclonal antibodies to a chemically synthesized peptide corresponding to the C-terminal mutant CALR neoepitope sequence (FIG. 1A). Out of 3 hybridoma screens, several antibodies showed positive binding to the peptide and to the CALR mutant protein in solution with clone 4D7 showing the strongest reactivity (FIG. 6A). By using peptides covering different regions of the immunizing neopeptide we mapped the binding of 4D7 to the 11 amino acids of the N-terminus with no binding of 4D7 observed to a scrambled peptide or the C-terminal portion of the immunogen (FIG. 1B). Clone 4D7 mAb showed a dissociation constant (Kd) of 1.53 nM to the full-length peptide as measured by I¹²⁵-Scatchard analysis (FIG. 1C and FIG. 6B). To investigate the specific binding of 4D7 on CALR mutant-bearing cells we generated cytokine independent TF-1 TpoR CALR^{del61} and TF-1 TpoR CALR^{del52} (FIG. 6C). Using these cells in a horizontal co-culturing system we found no evidence of a paracrine effect by the mutant CALR protein (FIG. 6D). Specific binding of phycoerythrin (PE)-conjugated 4D7 to the cell surface of mutant CALR-expressing cells, but not wild type TF-1 TpoR expressing cells, was observed in comparison to unstained or isotype-PE control (FIG. 1D, FIG. 6E).

### Monoclonal Antibody 4D7 Blocks TPO-Independent Signaling

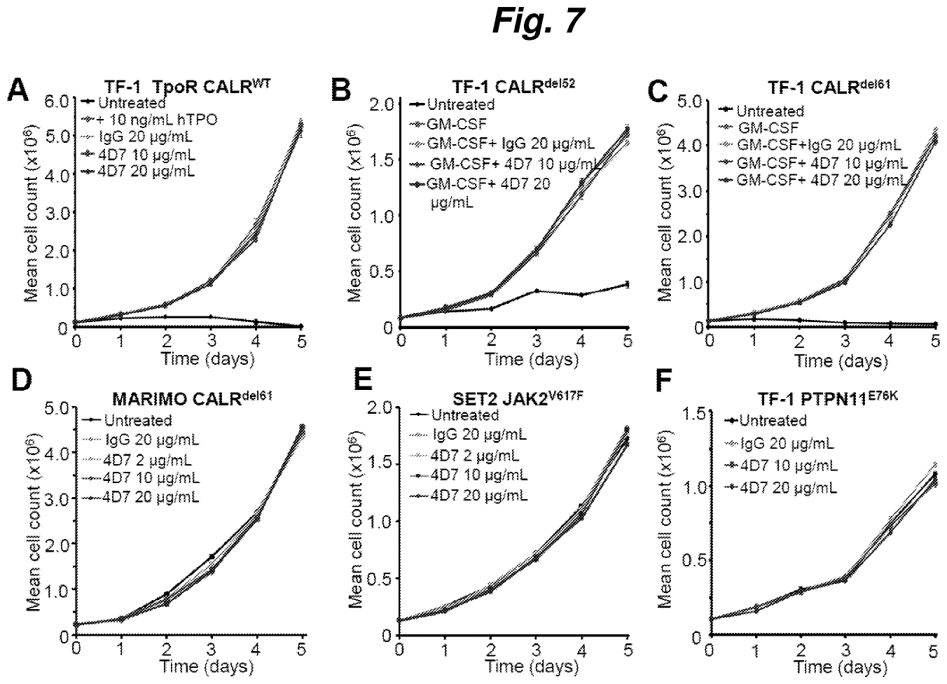

We then tested cytokine independent TF-1 TpoR CALR^{del61} and TF-1 TpoR CALR^{del52} cells for their ability to proliferate after treatment with increasing concentrations of 4D7 antibody or isotype control. Firstly, we noted no inhibition by 4D7 of TF-1 cells expressing TpoR alone (with endogenous wild type CALR) (FIG. 1E). In contrast, we observed blockade of TPO-independent cell growth over 5 days at 2, 10 and 20 μg/mL 4D7 (FIG. 1F and G) with evidence of concentration-dependent inhibition observed after 48 hours. No inhibition was observed with IgG isotype control antibody at 20 μg/mL. No inhibition by 4D7 was observed in TF-1 cells with overexpression of wild type CALR (FIG. 7A). Additionally, TF-1 cells harboring either the CALR^{del52} or CALR^{del61} mutations, but lacking TpoR were unaffected when treated with 4D7 (FIG. 7B and C). Interestingly, MARIMO cells also did not show inhibition with 4D7, consistent with CALR no longer acting as a driver mutation in this NRAS mutant cell line (Han et al, 2018) (FIG. 7D). In addition, no inhibition was observed in factor-independent JAK2 V617F SET2 cells (FIG. 7E) or factor-independent TF-1 expressing the PTPN11 E76K mutation (FIG. 7F) indicating a mutation-specific effect.

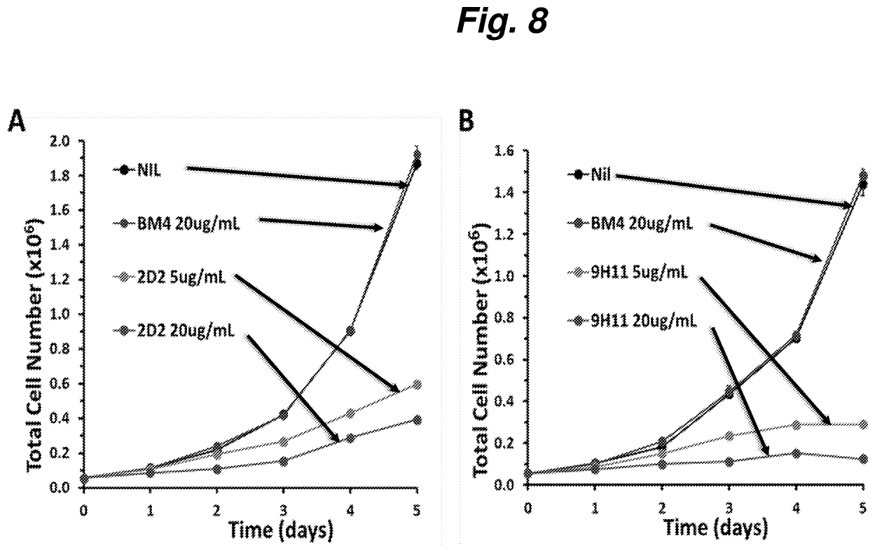

FIG. 8 shows proliferation curves of factor-independent TF-1 TpoR CALR^{del61} cells cultured with 2D2 or 9H11. A. Proliferation curves of factor-independent TF-1 TpoR CALR^{del61} cells cultured with 5 or 20 μg/mL of 2D2 or 20 μg/mL of control IgG antibody (n=3 biological replicates). B. Proliferation curves of factor-independent TF-1 TpoR CALR^{del61} cells cultured with 5 or 20 μg/mL of 9H11 or 20 μg/mL of control IgG antibody (n=3 biological replicates).

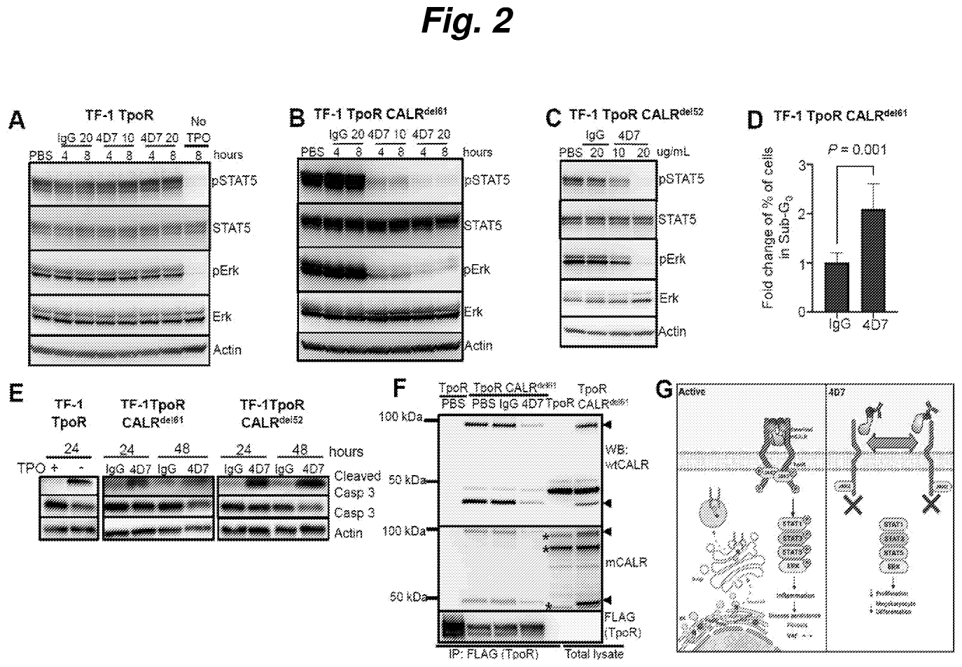

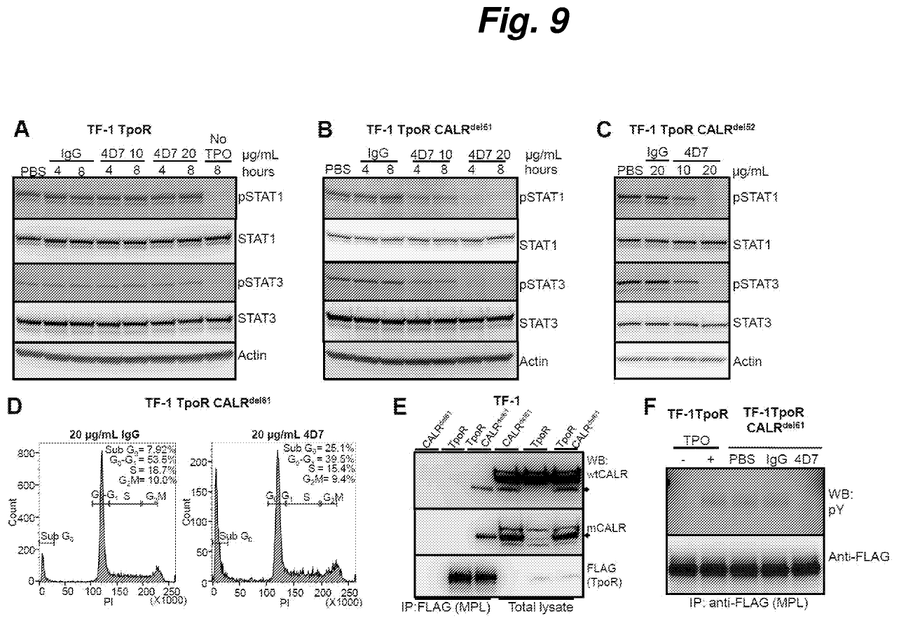

To understand the mechanism of action of 4D7, we performed signalling experiments in TF-1 cells. Firstly, we observed no inhibition of signalling in cytokine dependent TF-1 cells expressing TpoR alone, which showed the expected down-regulation of phospho-STAT1,3,5 and phospho-ERK after TPO withdrawal (FIG. 2A and FIG. 9A). In contrast, we observed blockade of constitutive factor-independent phospho-STAT1,3,5 and phospho-ERK after incubation with 10 or 20 μg/mL 4D7, but not with IgG control in both CALR^{del61} and CALR^{del52} cells (FIG. 2B and C, FIG. 9B and C). A significant increase in the sub-Go fraction was observed by 4D7 compared to IgG control (P=0.001, n=3 independent experiments) (FIG. 2D) in the absence of a major effect on G2-M on cell cycle analysis (FIG. 9D) consistent with induction of an apoptotic response. This was confirmed by caspase 3 cleavage that occurred within 24 hours, and peaked at 48 hours after 4D7 treatment (FIG. 2E).

### 4D7 Disrupts Binding of Mutant CALR to TpoR

To investigate the mechanism of action of 4D7 we examined how it affects mutant CALR interactions with TpoR. Analysis of TF1 TpoR CALR^{del61} cells revealed that CALR mutants constitutively existed in complex with the TpoR (FIG. 7E) consistent with previous findings (Araki et al., 2019). Interestingly, in these cells experiments under non reducing conditions revealed that mutated CALR was present in two forms, disulfide-linked dimers and monomers. These monomers and dimers were recognized by anti-mutant CALR (Dianova) antibody as well as an anti-wild type CALR antibody (Cell Signaling) directed to a central sequence of the CALR protein (arrowheads, FIG. 2F). No dimers of CALR bound to TpoR were observed under non-reducing (FIG. 2F) or reducing (FIG. 9E) conditions. Importantly, pre-treatment with 4D7 diminished the presence of CALR disulfide-linked dimers as well as of CALR monomers bound to TpoR (FIG. 2F) supporting the notion that 4D7 interferes with the multimerisation of mutant CALR/TpoR complexes at the cell surface either by preventing the disulfide linkage of CALR mutant proteins, by disrupting the constitutive association of CALR mutants to TpoR, or both (FIG. 2G). Similar results were obtained with TF-1 TpoR CALR^{del52} (FIG. 10A). Regardless of the exact molecular mechanism it is clear that 4D7 prevents TpoR activation in the presence of CALR mutant protein as demonstrated by its inhibition of TpoR tyrosine phosphorylation (FIG. 9F) and downstream signaling (FIG. 2B and C).

### Monoclonal Antibody 4D7 Antibody Inhibits TPO-Independent Megakaryocyte Formation

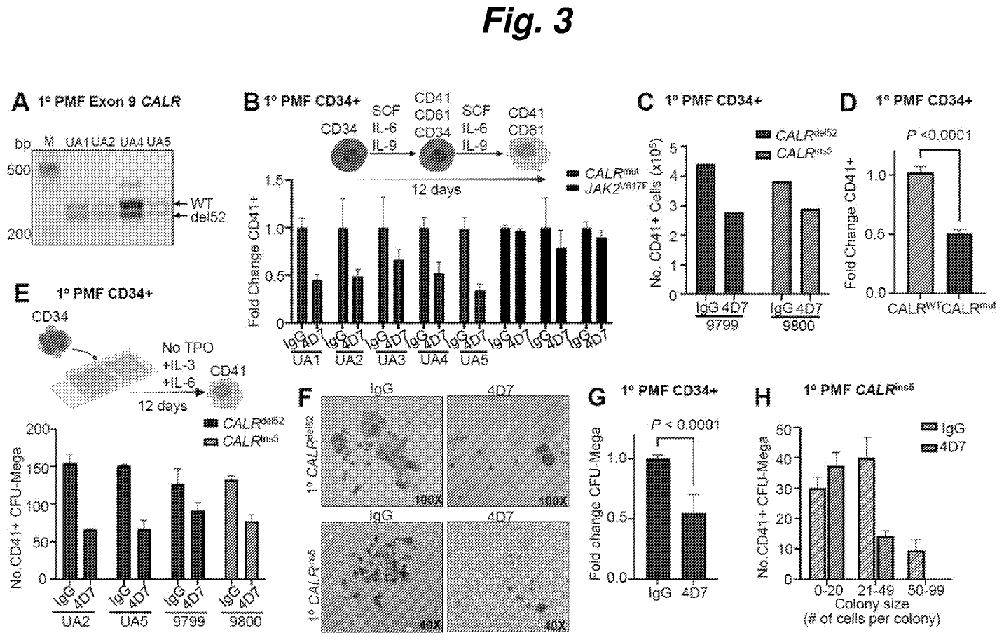

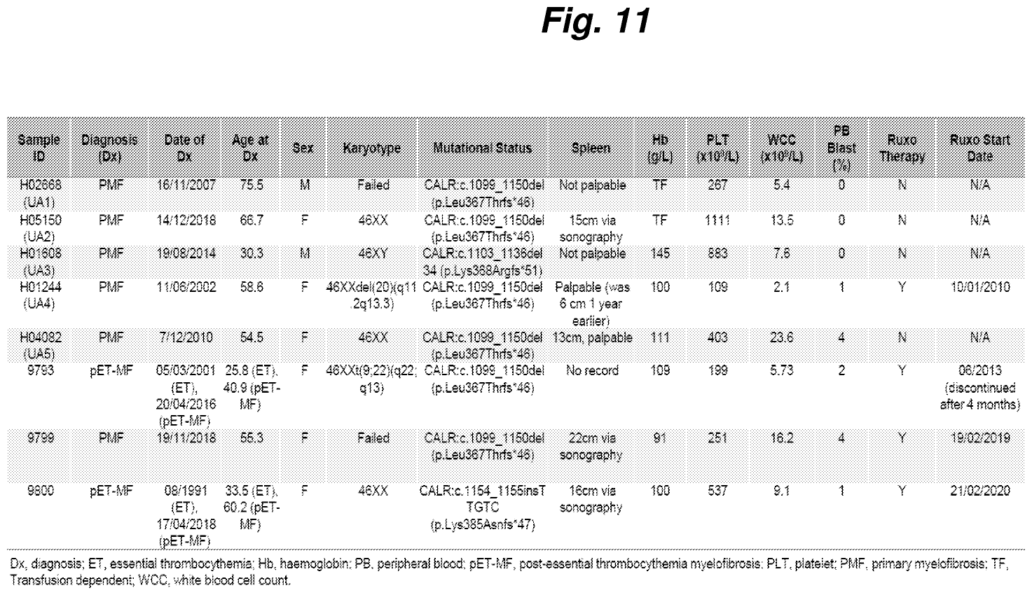

PMF is characterized by abnormal proliferation and morphology of clonal megakaryocytes. To examine whether 4D7 suppressed CALR mutant megakaryocytes, we tested its activity on purified primary CD34+ cells obtained from patients with CALR mutant myelofibrosis using two orthogonal assays: (i) TPO-independent megakaryocyte differentiation in liquid culture, and (ii) TPO-independent megakaryocyte colony formation on a collagen-based medium. Five out of 8 patients had the most common, type 1 (52 base pair deletion) CALR mutation which was heterozygous in the bulk of the CD34+ fraction (FIG. 3A). The sorting scheme for myelofibrosis stem cells is shown in FIG. 10B and clinical details for all patients are listed in FIG. 11. Four out of 5 mutant CALR MF patient samples that displayed robust TPO-independent growth of CD41+CD61+megakaryocyte progenitors showed inhibition by 4D7 of at least 50% (FIG. 3B, FIG. 10C). Interestingly, the patient sample with a weaker inhibition harboured a 34 base pair deletion. No significant inhibition was observed when JAK2 V617F was present in 3 out of 3 myelofibrosis samples (black bars: FIG. 3B). Additional samples in 2 independent patients obtained from the University of Graz, Austria, also showed evidence of inhibition compared to IgG control, including a type 2 CALR patient (5 base pair insertion) (FIG. 3C). Overall, a mean decrease of 55% was observed across CALR mutant samples after 4D7 treatment (P<0.0001. Student's t-test. n=6 independent samples) (FIG. 3D). Similarly. we saw a dramatic reduction in the absolute numbers of primary TPO-independent megakaryocyte colonies cultured on collagen (colony-forming unit-mega) treated with 4D7 in multiple patient samples (decrease of 46%. P<0.0001. Student's t-test, n=4 independent patient samples) (FIG. 3E-G). Residual colonies were still positive for CALR mutation. Strikingly, the size of 4D7 treated megakaryocyte colonies was substantially smaller as enumerated in a patient with a type 2 mutation (FIG. 3H).

### Monoclonal Antibody 4D7 Inhibits the Growth of Ruxolitinib Persistent Cells

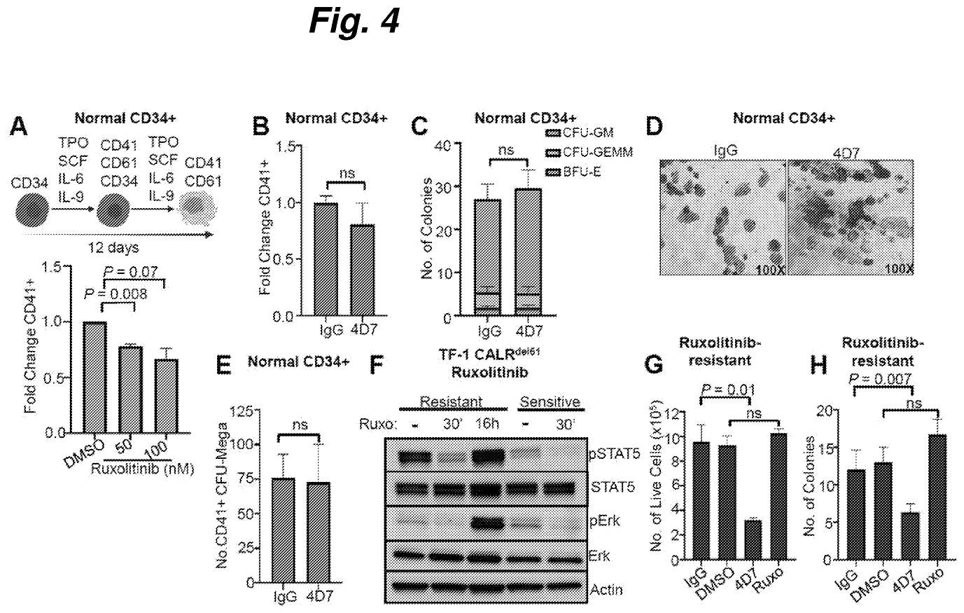

Ongoing follow-up of COMFORT-1 and COMFORT-2 studies (Harrison et al. 2012; Verstovsek et al. 2012) suggest patients with CALR mutations are less responsive to the JAK inhibitor ruxolitinib than patients with JAK2 mutations, but cytopenia limits the use of higher treatment doses (Ross et al., 2021). We therefore tested whether an immunotherapeutic approach would have efficacy in patients with documented ruxolitinib “persistence/resistance”. We first showed that ruxolitinib had inhibitory effects on megakaryocyte formation from healthy cord blood with >30% inhibition after 12 days at 50 or 100 nM (P=0.008 and 0.07 respectively) (FIG. 4A). In contrast, 4D7 had no effect on normal hematopoiesis. At the highest concentration, 4D7 did not inhibit physiological megakaryopoiesis in liquid culture (FIG. 4B), hematopoietic granulocyte-macrophage colony formation and erythroid colony formation (FIG. 4C), nor collagen-plated megakaryocyte colony numbers (CFU-Mega) (FIG. 4D and E) compared to IgG control.

To generate ruxolitinib-resistant cells, we cultured CALR mutant TPO-independent TF-1 cells with increasing concentrations of ruxolitinib over 4 weeks, up to 100 nM. Ruxolitinib-resistant cells showed enhanced phospho-STAT5 and phospho-ERK phosphorylation in the presence of ruxolitinib but still showed some inhibition after withdrawal and re-stimulation (FIG. 4F). Importantly, 4D7 had strong inhibitory activity on cells that were resistant to ruxolitinib, in both liquid culture at 96 hours (FIG. 4G) and in colony formation (FIG. 4H). Together, these results suggest that an immunotherapeutic approach with 4D7 may have clinical utility in CALR mutant patients who develop resistance/persistence of myelofibrosis during ruxolitinib treatment.

### The 4D7 Monoclonal Antibody Blocks CALR-Dependent Myeloid Cell Proliferation and Prolongs Survival in Xenograft Models

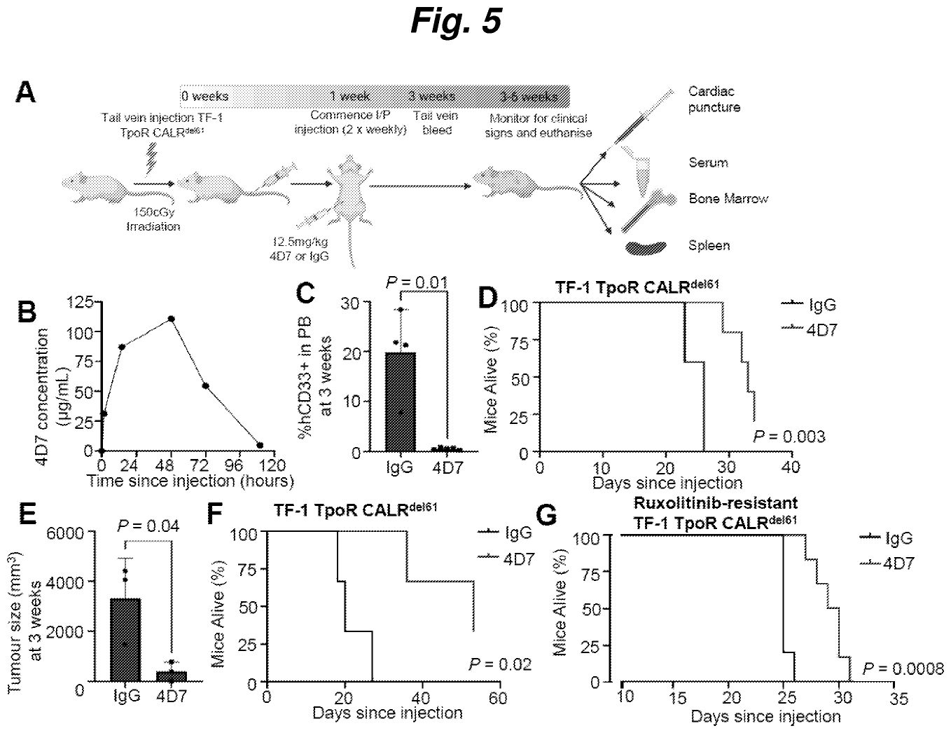

Myeloproliferative neoplasms are fundamentally disorders of unregulated proliferation. To test whether 4D7 could block mutant CALR-dependent proliferation in vivo, we developed two distinct xenograft models. The first was a bone marrow engraftment model, which measures mutant CALR-dependent proliferation in the bone marrow microenvironment. The second was a chloroma model created by subcutaneous injection of TPO-independent TF-1 TpoR CALR^{del61} cells into the flanks of NSG mice, which mimics extramedullary hematopoesis. In both models, fibrosis is not an evaluable disease outcome. In the bone marrow engraftment model (FIG. 5A), 4D7 treatment (12.5 mg/kg twice weekly via intraperitoneal injection starting day 7) showed an excellent pharmacokinetic profile, achieving a serum concentration of more than 100 μg/mL 48 hours post injection (FIG. 5B), lowered peripheral blood engraftment of human CD33+myeloid cells at 3 weeks (0.04 vs 19.8% CD33 4D7 vs IgG, P=0.01) (FIG. 5C), and significantly prolonged survival (log-rank hazard ratio 0.24, P=0.003) (FIG. 5D). In the chloroma model, 4D7 treatment significantly slowed tumour growth at 21 days post-engraftment (353 vs 3317 mm³ mean tumour volume, 4D7 vs IgG, P=0.04) (FIG. 5E) and prolonged survival (hazard ratio 0.19, P=0.02) (FIG. 5F). Strikingly, mutant CALR cells induced to be resistant to 100 nM ruxolitinib also showed a survival advantage after treatment with 4D7 (12.5 mg/kg twice weekly beginning at day 7) in the bone marrow engraftment model (hazard ratio 0.26, P=0.0008) (FIG. 5G). Together, these results suggest an immunotherapeutic approach with antibody 4D7 may have clinical utility in CALR-driven myeloproliferative neoplasms as well as in CALR mutant patients that develop resistance/persistence to ruxolitinib.

PMF is an insidious and poorly understood disorder that encompasses features of both cancer and chronic inflammation. It is a clonal neoplasm driven by a handful of somatic mutations that activate cell signalling, presumably residing in the long-term stem cell compartment (Nangalia et al., 2013: Reinisch et al, 2016; Wernig et al, 2006), but it also has a constellation of cytokine-mediated symptoms that are disproportionately severe. After the discovery of activating JAK2 mutations in 50-60% of patients with PMF (Baxter et al, 2005: Kralovics et al, 2005), clinicians were hopeful that myelofibrosis would respond to tyrosine kinase inhibitor therapy in a similar fashion to chronic myeloid leukemia. However, maturing data from the COMFORT studies and other trials have shown ruxolitinib also does not cause widespread regression of fibrosis nor eradicate disease clones in a mutation-specific manner (Cervantes et al, 2013: Harrison et al, 2016: Verstovsek et al., 2012). Exceptional cases of complete hematologic or molecular remission have been reported (Masarova et al, 2016), but are uncommon. Typically, there is a gradual loss of response over time, with only 27% of patients remaining on first-line ruxolitinib treatment after 5 years in the COMFORT studies, underscoring a need for alternative approaches.

Somatic mutations in exon 9 of the endoplasmic chaperone protein CALR are found in 70-80% of patients with JAK2-negative MF, accounting for ˜30% of MF cases overall, and also present at in a similar proportion of essential thrombocythemia (Klampfl et al., 2013: Nangalia et al., 2013). In a subsequent analysis of patients from the COMFORT-2 study the response rate to ruxolitinib was 20% (confidence interval 5.7-43.7%) in patients with a CALR mutation versus 34% (24.8-44.1%) in CALR-negative patients, most of whom had a JAK2 V617F mutation (Guglielmelli et al, 2016), suggesting that CALR-mutant myelofibrosis may be less responsive to JAK inhibitor therapy. Although more than 50 CALR mutations have been described, the majority can be classified as type 1-like or type 2-like based on predicted protein structures (Eder-Azanza et al, 2014). Our data suggest that both type 1 and type 2 CALR mutations could be amenable to an immunotherapeutic approach.

Here we show for the first time, that primary cells from patients with CALR mutations can be targeted with a mutation-selective immunotherapy. Our data confirm the observations from other studies using human cells, that mutant CALR protein is secreted and accessible on the cell surface and can drive growth factor independence, provided TpoR is co-expressed. Data from independent laboratories have shown that mutant CALR protein requires TpoR for signalling and factor-independent cell growth, and that the normal lectin domain of CALR is essential to bind glycosylated sites on TpoR (Elf et al. 2018: Elf et al., 2016; Pecquet et al, 2019). The relative contribution of autocrine vs paracrine vs endosomal signalling of mutant CALR protein has not been fully examined, with most studies to date performed in murine IL-3 dependent Ba/F3 cells. In our human TF-1 model of mutant CALR-induced proliferation, no evidence of a paracrine effect could be demonstrated in semi-porous transwells (FIG. 6D). Remarkably, despite a demonstrated lack of autocrine/paracrine signalling, mutant CALR protein appears to be exquisitely sensitive to 4D7 binding in a dose-dependent manner with concomitant inhibition of cell proliferation. Our results indicate that the CALR-TpoR complex can be disrupted via a monoclonal antibody, and specifically we identified evidence for disulfide linked mutant CALR bound constitutively to TpoR that was disrupted by 4D7, thus blocking TpoR phosphorylation. Future studies should determine the 3D structure of 4D7 bound to CALR dimers and help test whether TPO analogs can overcome the effects that we describe (Basso-Valentina et al, 2021).

Recently it was reported that mutant CALR protein is present at high levels in the plasma of myelofibrosis patients (compared to normal individuals) (Pecquet et al, 2018: Sollazzo et al, 2016). The cell surface expression of mutant CALR is an ideal target for immunological therapies. It is present on the cell surface (Elf et al., 2018: Elf et al., 2016) and contains sequences not normally present in healthy mammalian cells nor conserved across mammalian species. CALR mutations appears to be an early event in MPN ontogeny (perhaps only preceded by TET2 in some cases). This results in it being present in all of the cells of the clone, unlike JAK2^{V617F} which can be either an initiating or a secondary lesion, and found in only around half of patients with AML arising from an antecedent JAK2^{V617F} MPN (Ross et al., 2021).

The development of a therapeutic antibody directed against mutant CALR protein has enormous clinical potential. The antibody that we have developed against the common neoepitope C-terminus of CALR can be used in both insertion and deletion CALR mutation positive patients and appears to have minimal or no effects on normal cells ex vivo. The safety and tolerability of this antibody therapy could lead to its application in the earlier stages of PMF to prevent clinical progression and the acquisition of clonal heterogeneity. In parallel, an anti-mutant CALR antibody such as 4D7 could also be combined with other treatments with the aim of augmenting their effect, as has been successfully applied in B-cell lymphoma using anti-CD20 antibodies (Czuczman et al, 1999). Future studies should examine a similar approach in patients with essential thrombocy themia with CALR mutations and secondary acute myeloid leukemia that has transformed from CALR mutant myeloproliferative disorders.

Myelofibrosis can be associated with immune defects which are compounded by the effects of JAK inhibitors like ruxolitinib. There is therefore an argument in favour of immunotherapeutics that can be used in the less advanced stages of myeloproliferative neoplasms. Although immunodeficiency may limit the potential of antibody therapeutics that require intact complement-mediated cytotoxicity this can be overcome by the addition of toxin conjugates or the use of chimeric antigen receptor T cells. Importantly, our data suggest that antibody-mediated inhibition of cell signalling may also contribute to suppression of CALR mutant cell proliferation, independent of any immune-mediated effects. Physiologically, the SIRP alpha protein is normally present as a ligand for the “don't eat me” CD47 signal protecting cells from phagocytosis (Liu et al., 2020: Majeti, 2011: Majeti et al, 2009) but inhibition of macrophage phagocytosis does appear to be involved in the disease progression of MPN (Daitoku et al, 2016). It will be intriguing to test anti-CALR monoclonal antibody efficacy in the setting of macrophage activation or CAR T cell therapies.

### Materials and Methods

### Cell Lines

TF-1 cells were cultured in RPMI with 10% (v/v) fetal calf serum (FCS) supplemented with 2 ng/ml of GM-CSF and 25 mM HEPES. HEK293T cells were cultured in DMEM with 10% FCS. TF-1 TpoR cells were cultured in RPMI with 10% FCS supplemented with 10 ng/ml of human TPO (Peprotech). MARIMO and SET2 cells were cultured in RPMI with 10% FCS.

### CALR Mutation Cloning

Human CALR (del61) with a 61 base pair deletion in CALR exon 9 was PCR amplified from MARIMO cDNA with oligonucleotide primers 5′-TAT AGA ATT CGC CAC CAT GCT GCT ATC CGT GCC G-3′ and 5′-TAT AGA ATT CAG GCC TCA GTC CAG CCC T-3′. The resultant PCR product was cloned into pCX4-IRES eGFP (Neubauer et al, 2016) following digestion with EcoRI. Sequencing verified integrity and orientation of the cDNA. CALR^{del61} protein was expressed in TF-1 TpoR. This cell line was designated as TF-1 TpoR CALR^{del61} and was shown to obtain cytokine independence.

### Rat Monoclonal Antibody Production

Rats were immunised with a CALR mutant peptide “MMRTKMRMRRMRRT RRKMRRKMSPARPRTS” coupled to KLH, this peptide sequence is unique to CALR mutant. Serum from the immunised rats was screened by enzyme linked immunoassay, to verify a strong titre to the peptide immunogen, prior to performing the hybridoma fusion. Immunised rat spleen cells were harvested and a spleen single cell suspension was combined with Sp2/O myeloma cell line. The cell mixture was treated with polyethylene glycol plus DMEM prior to been plated in 96 well flat bottom plates. The expanding hybridomas were screened initially by ELISA and the positive clones were further verified by western blot, for reactivity to the expressed CALR mutant protein before subcloning the hybridomas for antibody production.

### Lentiviral Expression Constructs

Human CALR and PTPN11 vectors were produced using a codon-optimised coding sequence encoding either CALR^{WT}, CALR^{del52} or PTPN11^{E76K}-mutations purchased as gBlocks from Integrated DNA Technologies (Iowa, USA). Mutations were cloned into pLVX-eF1α-IRES-ZsGreen1 lentiviral expression construct (Takara Bio Inc, Shiga, Japan). For virus production, HEK293T cells were seeded at 3×10⁶ cells per 75 cm² flask 40 hours prior to transfection. Each flask was transfected using Lipofectamine 2000 with 13.5 μg of CALR WT or mutant pLVX vectors together with 13 μg envelope plasmids, VsVg and PAX2. Viral supernatants were collected 48 hours after transfection. To establish cell lines expressing various CALR constructs, viral supernatants were applied to RetroNectin-coated (Takara) tissue culture plates and spun at 2000×g for 2 hours at 37° C. Cell lines were applied to virus and spun at 200×g rpm and cultured in the presence of virus for 24 hours. ZsGreen-positive cells were sorted using the FACS Melody (BD Biosciences).

### Antibody ¹²⁵Iodine-Labelling and Scatchard Analysis

4D7 was radio-iodinated with ¹²⁵I (Perkin-Elmer) using Pierce Pre-Coated Iodination tubes (Thermo Scientific) with an estimated specific activity of 7733cpm/ng. For saturation binding assays, plates were coated overnight at 4° C. with full-length neoepitope CALR peptide (2 μg/mL) or control scrambled peptide (2 μg/mL). Wells were washed 3 times with phosphate buffered saline+0.05% Tween20, blocked with 5% bovine serum albumin for 1 hour followed by further washing. ¹²⁵I-4D7 was added at concentrations ranging from 2 pM to 2.5 nM in triplicate and incubated at room temperature for 2 hours. After washing, 1M HCl was added for 30 minutes to elute bound antibody. 200 μL aliquots from each well were counted on a PerkinElmer 2470 Wizard2 Automatic Gamma Counter. Dissociation constants were calculated using the EBDA and LIGAND programs.

### Cell Proliferation to Assess 4D7 Effect on Various Cell Lines.

In brief, cells were seeded at 5×10⁴/mL in standard growth media, with any necessary cytokines, and the addition of 20 μg/mL IgG or 2, 10 or 20 μg/mL 4D7. Cells were seeded in triplicate and each well was counted in triplicate every 24 hours for 5 days using trypan blue exclusion.

### Primary Myelofibrosis Stem Cell Experiments

Umbilical cord blood (UCB) was collected with written consent from full-term deliveries at the Women's Health Unit, Lyell McEwin Hospital (Adelaide, South Australia) or the Department of Obstetrics and Gynecology, Medical University of Graz (Austria) with institutional review board approval (IRB approval: 31-322 ex 18/19; HREC/20/WCHN/65). Samples were processed using a Ficoll-Paque (GE Healthcare) density gradient to isolate mononuclear cells (800×g, room temperature, 30 minutes, deceleration off), followed by red cell lysis (ammonium-chloride-potassium lysing buffer) to remove remaining red blood cells. CD34 enrichment was performed by magnetic cell separation using CD34 Microbead kit (Miltenyi Biotech). Alternatively, purified CD34+ cells from cord-blood were purchased from Lonza. CD34+ cells with a purity above 90% were either cryopreserved or directly cultured in serum-free stem cell retention media (StemSpan SFEMII, Stemcell Technologies, Vancouver, BC, Canada) supplemented with human recombinant SCF, TPO, FLT3, IL-6 (all 20 ng/mL), UM729 (1.75 uM) and StemReginin 1 (SR1, 300 nM). All cytokines and SRI were purchased from PeproTech (Rocky Hill, NJ, USA) whereas UM729 was purchased from Stemcell Technologies.

### PMF Patient Samples

Patient samples were collected with informed consent from the South Australian Cancer Research Biobank (SACRB: Adelaide, Australia). Primary CD34+ stem/progenitor cells from peripheral blood samples from patients with JAK2^{V617F} or CALR mutant myelofibrosis were purified by flow cytometry using CD34-APC antibody, anti-human CD45RA Brilliant Violet 605, anti-CD90 FITC, anti-human CD123-PE, anti-human CD38 PE-Cy7, or with CD34-APC antibody, anti-human CD3 FITC, anti-human CD19-PE (clone H1B19) and anti-human CD14 PE-Cy7.

### Mutational Screening of Patient Samples

DNA was extracted from primary PMF samples using QuickExtract™ (Epicentre Biotechnologies) following the manufacturers protocol. PCR reactions were set up in a final concentration of 1 X PCR buffer, 1.5 mM MgCl₂, 0.2 mM dNTPs, 0.2 uM forward and reverse primers, (CALR-F: 5′-TAA CAA AGG TGA GGC CTG GT-3′, CALR-R: 5′ GCC TCT CTA CAG CTC GTC CTT-3′), 0.5 units Taq Polymerase (Promega) and 50 ng genomic DNA in a final volume of 25 uL. Reactions were undertaken in a T100 Thermocycler (BioRad). Samples were activated/denatured at 95° C. for 10 minutes, then were cycled at 95° C. for 30 seconds, 58° C. for 30 seconds and 72° C. for 1 minute for 40 cycles, followed by a 10 minute incubation at 72° C. Samples were visualised by electrophoresis on a 2% agarose gel containing 1 X Gel Red (Biotium) and imaged on the BioRad GelDoc XR+ (BioRad).

### Hematopoietic Colony Formation

For megakaryocyte differentiation assays, CD34+ cells derived from cord blood or from patient peripheral blood mononuclear fractions were plated in Megacult (MegaCult™-C Collagen and Medium with Lipids) with added IL-3 and IL-6 (10 ng/ml) in the presence or absence of 50 ng/mL TPO. Cells were plated at an initial density of 1800 cells/well and cultured for 12 days at 37° C. and 5% CO₂. Primary PMF CD34+ cells were cultured in StemCell Pro supplemented with human recombinant SCF (25 ng/mL), IL-6 and IL-9 (10 ng/ml) to allow differentiation into megakaryocytes. CD34+cord blood cells were cultured in 50 ng/mL SCF, 50 ng/ml TPO, 20 ng/ml IL-6 and 20 ng/ml IL-9. Both IgG and 4D7 were added at 20 μg/mL. All cytokines are from Preprotech. Culture media was replenished every 3 days and cells were assessed for megakaryocyte differentiation on day 12 using anti-human CD41/61 PE-Cy7 (Clone A2A9/6) and CountBright beads (Thermo Fisher) to enumerate cell numbers. CD34+obtained from UCB were plated in MethoCult containing cytokines (H4434). Cells were seeded at approximately 100 cells per well and were cultured in the presence of 20 μg/mL 4D7 or control IgG antibody and scored for colony and blast forming units at 12-14 days.

### Flow Cytometry Assessment of 4D7 Binding to the Cell Surface

TF-1 cells expressing TpoR and mutant CALR were incubated for 30 minutes 4D7 conjugated to phycoerthryin (PE) (Abcam, #AB102918) or IgG2a PE isotype control (ThermoFisher #12-4321-42) at the concentrations specified after pre-incubation with mouse serum. Cells were washed 1 × before analysis on a CytoFLEX (Beckman Coulter).

### Analysis of Cell Cycle

Cell cycle analysis was carried out by flow cytometry. Briefly, TF-1 TpoR and TF-1 TpoR CALR^{del61} cells were seeded at a concentration of ˜2×10⁵ per well in triplicate under standard culture conditions, with the addition of 20 μg/mL IgG or 4D7. Cells were treated for 48 hours and were fixed by 70% (v/v) ethanol at 4° C. overnight. Fixed cells were rinsed twice with PBS and stained with 50 μg/mL propridium iodide (PI: ThermoFisher)+100 μg/mL RNAse A (QIAGEN). Cells were incubated for 30 minutes at RT in the dark and analysed on the BD FACSCantoII (BD Biosciences).

### Co-Culture of CALR Secreting Cells.

In brief, TF-1 TpoR and TF-1 TpoR CALR^{del61} were seeded at the same density in opposing wells of a UniWells™ Horizontal co-culture plate (FujiFilm, #2501-02FW) separated by a 0.75 μm filter. Cells were seeded in the presence or absence of 10 ng/ml TPO in triplicate and counted every day for 4 days by trypan blue exclusion. Representative images of wells were taken on day 4.

### Protein Lysate Preparation and Western Blot Analysis

For immunoblotting of total lysates, cells were lysed in NP40 buffer containing 150 mM NaCl, 50 mM Tris pH 7.6, 1% NP-40, supplemented with protease inhibitors (Complete, Roche) and phosphatase inhibitor cocktails, and boiled for 5 minutes after addition of sample buffer (60 mM Tris pH 6.8, 5% glycerol, 1% SDS, 2% β-mercaptoethanol, 0.02% bromophenol blue) before SDS gel electrophoresis followed by Western blotting (Tvorogov et al, 2010). Primary antibodies against pERK (#9101), pSTAT5 (#9359), ERK (#4695), Actin (#4970), Caspase 3 (#14220), wildtype CALR (#12238) were purchased from Cell Signalling. Primary antibodies against pSTATI (#612233), pSTAT3 (#612357), STATI (#610186), STAT3 (#610190), STAT5 (#610192) and Anti-phosphotyrosine 4G10 (#610012) were purchased from BD Biosciences. Mutant CALR monoclonal antibody CAL2 was purchased from Dianova (Hamburg, Germany). Immunopreciptation of TpoR was performed in standard NP40 lysis buffer with additional 50 mM iodoacetamide to avoid any de novo disulfide bond formation post lysis. To reduce IgG background during immunoprecipitation anti-FLAG conjugated to magnetic beads (Sigma, #M8823) were used. For this same reason, anti-FLAG-HRP conjugated antibodies (Sigma, #A8592) were used in western blotting.

### Ruxolitinib-Resistant Cells

Ruxolitinib-resistant TF-1 TpoR CALR^{del61} cells were established by exposing cells of untreated TF-1 TpoR CALR^{del61} cells to increasing concentrations of ruxolitinib (Selleckchem) over a 4 week period. Cells were initially treated with 10 nM and increased to 100 nM.

### Xenograft Models of Disease

All procedures were approved by the South Australian Health and Medical Research Animal Ethics Committee (Protocol SAM21-018). Bone marrow engraftment model: 4-6 week old NSG mice were x-ray irradiated with 150 cGy and 5×10⁵ cells (TF-1-TpoR CALR^{del61}) or 5×10⁴ cells (100 nM ruxolitinib resistant TF-1 TpoR CALR^{del61}) were intravenously injected into mice. Seven days after injection, mice were administered 12.5 mg/kg IgG or 4D7 by intraperitoneal injection twice weekly. Tail vein bleeds were taken at 3 weeks and analysed via flow cytometry to determine human leukaemia content in the peripheral blood. Leukemic cells were PI-, mCD45.1, hCD33+, zsGreen+. Mice were monitored daily and euthanized once clinical symptoms were observed. At the time of euthanasia, cardiac puncture was performed to obtain both peripheral blood and serum for analysis. Additionally, bone marrow from both femurs, spine and spleens were analysed for leukemic content as detailed above. Chloroma model: 4-6 week old NSG mice were anaesthetised by isoflurane inhalation and shaved on the left back/flank and 1×10⁷ cells were injected into the mice. Cells were pre-treated with 20 μg/mL IgG or 4D7 for 1 hour prior to injection. Seven days after injection, mice were administered 12.5 mg/kg IgG or 4D7 by intraperitoneal injection twice weekly. Tumour progression was assessed through caliper measurements every 2 days. Mice were euthanised once tumours began to impede movement, show signs of ulceration or if any measurement of the tumour exceeded 30 mm (length, width or depth).

### REFERENCES

Araki M, Yang Y, Imai M, Mizukami Y, Kihara Y, Sunami Y, Masubuchi N, Edahiro Y, Hironaka Y, Osaga S et al (2019) Homomultimerization of mutant calreticulin is a prerequisite for MPL binding and activation. Leukemia 33:122-131
Araki M, Yang Y, Masubuchi N, Hironaka Y, Takei H, Morishita S, Mizukami Y, Kan S, Shirane S, Edahiro Y et al (2016) Activation of the thrombopoietin receptor by mutant calreticulin in CALR-mutant myeloproliferative neoplasms. Blood 127:1307-1316
Baade P D, Ross D M, Anderson L A, Forsyth C, Fritschi L (2019) Changing incidence of myeloproliferative neoplasms in Australia, 2003-2014. Am J Hematol 94: E107-E109
Basso-Valentina F, Levy G, Varghese L N, Oufadem M, Neven B, Boussard C, Balayn N, Marty C, Vainchenker W, Plo I et al (2021) CALR mutant protein rescues the response of MPL p.R464G variant associated with CAMT to eltrombopag. Blood 138:480-485
Baxter E J, Scott L M, Campbell P J, East C, Fourouclas N, Swanton S, Vassiliou G S, Bench A J, Boyd E M, Curtin N et al (2005) Acquired mutation of the tyrosine kinase JAK2 in human myeloproliferative disorders. Lancet 365:1054-1061
Cervantes F, Vannucchi A M, Kiladjian J J, Al-Ali H K, Sirulnik A, Stalbovskaya V, McQuitty M, Hunter D S, Levy R S, Passamonti F et al (2013) Three-year efficacy, safety, and survival findings from COMFORT-I I, a phase 3 study comparing ruxolitinib with best available therapy for myelofibrosis. Blood 122:4047-4053
Chachoua I, Pecquet C, El-Khoury M, Nivarthi H, Albu R I, Marty C, Gryshkova V, Defour J P, Vertenoeil G, Ngo A et al (2016) Thrombopoietin receptor activation by myeloproliferative neoplasm associated calreticulin mutants. Blood 127:1325-1335
Czuczman M S, Grillo-López A J, White C A, Saleh M, Gordon L, LoBuglio A F, Jonas C, Klippenstein D, Dallaire B, Varns C (1999) Treatment of patients with low-grade B-cell lymphoma with the combination of chimeric anti-CD20 monoclonal antibody and CHOP chemotherapy. J Clin Oncol 17:268-276
Daitoku S, Takenaka K, Yamauchi T, Yurino A, Jinnouchi F, Nunomura T, Eto T, Kamimura T, Higuchi M, Harada N et al (2016) Calreticulin mutation does not contribute to disease progression in essential thrombocythemia by inhibiting phagocytosis. Exp Hematol 44:817-825 e813
Eder-Azanza L, Navarro D, Aranaz P, Novo F J, Cross N C, Vizmanos J L (2014) Bioinformatic analyses of CALR mutations in myeloproliferative neoplasms support a role in signaling. Leukemia 28:2106-2109
Elf S, Abdelfattah N S, Baral A J, Beeson D, Rivera J F, Ko A, Florescu N, Birrane G, Chen E, Mullally A (2018) Defining the requirements for the pathogenic interaction between mutant calreticulin and MPL in MPN. Blood 131:782-786
Elf S, Abdelfattah N S, Chen E, Perales-Paton J, Rosen E A, Ko A, Peisker F, Florescu N, Giannini S, Wolach O et al (2016) Mutant Calreticulin Requires Both Its Mutant C-terminus and the Thrombopoietin Receptor for Oncogenic Transformation. Cancer Discov 6:368-381
Guglielmelli P, Rotunno G, Bogani C, Mannarelli C, Giunti L, Provenzano A, Giglio S, Squires M, Stalbovskaya V, Gopalakrishna P et al (2016) Ruxolitinib is an effective treatment for CALR-positive patients with myelofibrosis. Br J Haematol 173:938-940
Han L, Czech J, Maurer A, Brummendorf T H, Chatain N, Koschmieder S (2018) Mutant NRAS Q61K is responsible for MAPK pathway activation in the MARIMO cell line and renders these cells independent of the CALR-MPL-JAK2-STAT5 pathway. Leukemia 32:2087-2090
Han L, Schubert C, Köhler J, Schemionek M, Isfort S, Brümmendorf T H, Koschmieder S, Chatain N (2016) Calreticulin-mutant proteins induce megakaryocytic signaling to transform hematopoietic cells and undergo accelerated degradation and Golgi-mediated secretion. Journal of Hematology & Oncology 9:45
Harrison C, Kiladjian J J, Al-Ali H K, Gisslinger H, Waltzman R, Stalbovskaya V, McQuitty M, Hunter D S, Levy R, Knoops L et al (2012) JAK inhibition with ruxolitinib versus best available therapy for myelofibrosis. N Engl J Med 366:787-798
Harrison C N, Vannucchi A M, Kiladjian J J, Al-Ali H K, Gisslinger H, Knoops L, Cervantes F, Jones M M, Sun K, McQuitty M et al (2016) Long-term findings from COMFORT-I I, a phase 3 study of ruxolitinib vs best available therapy for myelofibrosis. Leukemia 30:1701-1707
How J, Hobbs G S, Mullally A (2019) Mutant calreticulin in myeloproliferative neoplasms. Blood 134:2242-2248
Klampfl T, Gisslinger H, Harutyunyan A S, Nivarthi H, Rumi E, Milosevic J D, Them N C, Berg T, Gisslinger B, Pietra D et al (2013) Somatic mutations of calreticulin in myeloproliferative neoplasms. N Engl J Med 369:2379-2390
Kralovics R, Passamonti F, Buser A S, Teo S S, Tiedt R, Passweg J R, Tichelli A, Cazzola M, Skoda R C (2005) A gain-of-function mutation of JAK2 in myeloproliferative disorders. N Engl J Med 352:1779-1790
Liu P, Zhao L, Loos F, Marty C, Xie W, Martins I, Lachkar S, Qu B, Waeckel-Énée E, Plo I et al (2020) Immunosuppression by Mutated Calreticulin Released from Malignant Cells. Mol Cell 77:748-760.e749
Majeti R (2011) Monoclonal antibody therapy directed against human acute myeloid leukemia stem cells. Oncogene 30:1009-1019
Majeti R, Chao M P, Alizadeh A A, Pang W W, Jaiswal S, Gibbs K D, Jr., van Rooijen N, Weissman I L (2009) CD47 is an adverse prognostic factor and therapeutic antibody target on human acute myeloid leukemia stem cells. Cell 138:286-299
Marty C, Pecquet C, Nivarthi H, El-Khoury M, Chachoua I, Tulliez M, Villeval J L, Raslova H, Kralovics R, Constantinescu S N et al (2016) Calreticulin mutants in mice induce an MPL-dependent thrombocytosis with frequent progression to myelofibrosis. Blood 127:1317-1324
Masarova L, Wang W, Newberry K J, Kantarjian H, Verstovsek S (2016) Complete remission in a patient with JAK2- and IDH2-positive myelofibrosis. Blood 128:877-880
Masubuchi N, Araki M, Yang Y, Hayashi E, Imai M, Edahiro Y, Hironaka Y, Mizukami Y, Kihara Y, Takei H et al (2020) Mutant calreticulin interacts with MPL in the secretion pathway for activation on the cell surface. Leukemia 34:499-509
Nangalia J, Massie C E, Baxter E J, Nice F L, Gundem G, Wedge D C, Avezov E, Li J, Kollmann K, Kent D G et al (2013) Somatic CALR mutations in myeloproliferative neoplasms with nonmutated JAK2. N Engl J Med 369:2391-2405
Neubauer H A, Pham D H, Zebol J R, Moretti P A, Peterson A L, Leclercq™, Chan H, Powell J A, Pitman M R, Samuel M S et al (2016) An oncogenic role for sphingosine kinase 2. Oncotarget 7:64886-64899
Passamonti F, Maffioli M, Merli M, Ferrario A, Caramazza D (2012) Clinical predictors of outcome in MPN. Hematol Oncol Clin North Am 26:1101-1116
Pecquet C, Balligand T, Chachoua I, Roy A, Vertenoeil G, Colau D, Fertig E, Marty C, Nivarthi H, Defour J P et al (2018) Secreted Mutant Calreticulins As Rogue Cytokines Trigger Thrombopoietin Receptor Activation Specifically in CALR Mutated Cells: Perspectives for MPN Therapy. Blood 132
Pecquet C, Chachoua I, Roy A, Balligand T, Vertenoeil G, Leroy E, Albu R I, Defour J P, Nivarthi H, Hug E et al (2019) Calreticulin mutants as oncogenic rogue chaperones for TpoR and traffic-defective pathogenic TpoR mutants. Blood 133:2669-2681
Reinisch A, Thomas D, Corces M R, Zhang X, Gratzinger D, Hong W J, Schallmoser K, Strunk D, Majeti R (2016) A humanized bone marrow ossicle xenotransplantation model enables improved engraftment of healthy and leukemic human hematopoietic cells. Nat Med 22:812-821
Rivera J F, Baral A J, Nadat F, Boyd G, Smyth R, Patel H, Burman E L, Alameer G, Boxall S A, Jackson B R et al (2021) Zinc-dependent multimerization of mutant calreticulin is required for MPL binding and MPN pathogenesis. Blood Adv 5:1922-1932
Ross D M, Babon J J, Tvorogov D, Thomas D (2021) Persistence of myelofibrosis treated with ruxolitinib: biology and clinical implications. Haematologica
Sollazzo D, Forte D, Polverelli N, Perricone M, Romano M, Luatti S, Vianelli N, Cavo M, Palandri F, Catani L (2016) Circulating Calreticulin Is Increased in Myelofibrosis: Correlation with Interleukin-6 Plasma Levels, Bone Marrow Fibrosis, and Splenomegaly. Mediators Inflamm 2016:5860657
Tvorogov D, Anisimov A, Zheng W, Leppänen V M, Tammela T, Laurinavicius S, Holnthoner W, Heloterä H, Holopainen T, Jeltsch M et al (2010) Effective suppression of vascular network formation by combination of antibodies blocking VEGFR ligand binding and receptor dimerization. Cancer Cell 18:630-640
Verstovsek S, Mesa R A, Gotlib J, Levy R S, Gupta V, DiPersio J F, Catalano J V, Deininger M, Miller C, Silver R T et al (2012) A double-blind, placebo-controlled trial of ruxolitinib for myelofibrosis. N Engl J Med 366:799-807
Wernig G, Mercher T, Okabe R, Levine R L, Lee B H, Gilliland D G (2006) Expression of Jak2V617F causes a polycythemia vera-like disease with associated myelofibrosis in a murine bone marrow transplant model. Blood 107:4274-4281

### Example 2—Additional High Affinity Anti-CALR Antibodies

We also tested the rat monoclonal antibodies developed after immunization with the 30 amino acid peptide corresponding to the C-terminal mutant CALR neoepitope sequence with an extra cysteine residue added as a linker (FIG. 1A). We identified two further antibodies, 2D2 and 9H11, which showed positive binding to peptide by ELISA and has superior activity of detecting mutant but not wild type CALR protein by Western blotting of cell extracts from TF-1 cells transduced with and without mutant CALR (data not shown).

FIG. 8 shows rat anti-human mutCALR 2D2, 9H11 antibody inhibit proliferation of cytokine-independent TF1 TpoR/mutCALR cells.

### Example 3—Sequencing of Antibodies

Sequencing of the heavy-chain variable regions (V H), and the light-chain variable regions (V L) of the monoclonal antibodies 4D7, 2D2 and 9H11. A summary of the methodology used to sequence the V H and V L regions is provided in FIG. 8.

Analysis of the V H and V L gene sequences was performed using the IMGT/V-Quest program, (The International Immunogenetics Information System; http://www.imgt.org/IMGT_vquest/vquest). The DNA and amino acid sequences of the V H and V L regions for 4D7 are shown in Figure and the annotated V H and V L amino acid sequences showing framework regions (FRs), and complementary determining regions (CDRs) in FIG. 10.

4D7 Heavy-Chain Variable Region (VH)
4D7 VH Nucleotide Sequence
(SEQ ID NO: 4)
CAGGTCCAAC TGCAGCAGTC TGGTGCTGAG CTGGCAAAGC CTGGCTCTTC AGTGAAAATT
TCCTGCAAGG CTTCTGGCTA CACCTTTACC AACTACGATA TGAGCTGGAT AAAGCAGAGG
CCTGGACAGG CCCTTGAGTG GATTGGAGCC ATTAATCCAG GAAATGGAGT TACAGACTAC
AATGAGAAGT TCAGGGGCAA GGCCACATTG ACTGTAGACA AATCCTCCAG CACAGCCTTC
ATGCAACTCA GCAGCCTGAC ACCTGAGGAC ACTGCGGTCT ATCACTGTGC AAGAGAATAT
AGGGACAACT ACGGCTACTT TGATTACTGG GGCCAAGGAG TCATGGTCAC AGTCTCCTCA
4D7 VH Amino Acid Sequence (SEQ ID NO: 5):
Gln Val Gln Leu Gln Gln Ser Gly Ala Glu Leu Ala Lys Pro Gly Ser
Ser Val Lys Ile Ser Cys Lys Ala Ser Gly Tyr Thr Phe Thr Asn Tyr
Asp Met Ser Trp Ile Lys Gln Arg Pro Gly Gln Ala Leu Glu Trp Ile
Gly Ala Ile Asn Pro Gly Asn Gly Val Thr Asp Tyr Asn Glu Lys Phe
Arg Gly Lys Ala Thr Leu Thr Val Asp Lys Ser Ser Ser Thr Ala Phe
Met Gln Leu Ser Ser Leu Thr Pro Glu Asp Thr Ala Val Tyr His Cys
Ala Arg Glu Tyr Arg Asp Asn Tyr Gly Tyr Phe Asp Tyr Trp Gly Gln
Gly Val Met Val Thr Val Ser Ser
4D7 Light-Chain Variable Region (VL)
4D7 VL Nucleotide Sequence (SEQ ID NO: 6):
GATATTGTGT TGACACAAAC TCCAGTTTCC CTGTCTGTCA CACTTGGAGA TCAAGCTTCT
ATATCTTGCA GGTCTAGTCA GAGCCTGGAA TATAGTGATG GATACACTTA TTTGGATTGG
TTCCTACAGA AGCCAGGCCA GTCTCCACAG CTCCTCATCT ATGAAGTTTC CAGCCGATTT
TCTGGGGTCC CAGACAGGTT CATTGGCCGT GGGTCAGGGA CAGATTTCAC CCTCAAGATC
AGCAGAGTAG AGCCTGAGGA CTTGGGAGTT TATTACTGCT TCCAAGCTAC ACATGATCCA
TTCACGTTCG GCTCAGGGAC GAAGTTGGAA ATAAAA
4D7 VL Amino Acid Sequence (SEQ ID NO: 7:
Asp Ile Val Leu Thr Gln Thr Pro Val Ser Leu Ser Val Thr Leu Gly
Asp Gln Ala Ser Ile Ser Cys Arg Ser Ser Gln Ser Leu Glu Tyr Ser
Asp Gly Tyr Thr Tyr Leu Asp Trp Phe Leu Gln Lys Pro Gly Gln Ser
Pro Gln Leu Leu Ile Tyr Glu Val Ser Ser Arg Phe Ser Gly Val Pro
Asp Arg Phe Ile Gly Arg Gly Ser Gly Thr Asp Phe Thr Leu Lys Ile
Ser Arg Val Glu Pro Glu Asp Leu Gly Val Tyr Tyr Cys Phe Gln Ala
Thr His Asp Pro Phe Thr Phe Gly Ser Gly Thr Lys Leu Glu Ile Lys

The results for 2D2 are shown in FIGS. 11 and 12.

2D2 Heavy-Chain Variable Region (VH)
2D2 VH Nucleotide Sequence
(SEQ ID NO: 8)
CAGGTCCAGT TGCAGCAGTC TGGAGCTGAG CTGACAAAGC CTGGCTCTTC AGTGAAGATT
TCCTGCAAGA CTTCTGGCTA CACCTTTACC ACCTACGATA TAAGCTGGAT AAAACAGAGG
CCTGGACAGG CCCTTGAGTG GATTGGAGCA ATCTATCCAG GAGGTGTAAC TACAGGCTAC
AATGAGAAGT TCAAGGGCAA GGCCACATTG ACTGTAGACA AATCTTCCAA CACGGCCTTC
ATGCAACTCA GCAGCCTGAC ACCTGAGGAC ACTGCGGTCT ATTACTGTGC AAGATGGGGT
CATACCACGG GTACCCGATT TGATCACTGG GGCCAAGGAG TCATGGTCAC AGTCTCCTCA
4D7 VH Amino Acid Sequence (SEQ ID NO: 9:
Gln Val Gln Leu Gln Gln ser Gly Ala glu Leu Thr Lys Pro Gly Ser
Ser Val Lys Ile Ser Cys Lys Thr Ser Gly Tyr Thr Phe Thr Thr Tyr
Asp Ile Ser Trp Ile Lys Gln Arg Pro Gly Gln Ala Leu Glu Trp Ile
Gly Ala Ile Tyr Pro Gly Gly Val Thr Thr Gly Tyr Asn Glu Lys Phe
Lys Gly Lys Ala Thr Leu Thr Val Asp Lys Ser Ser Asn Thr Ala Phe
Met Gln Leu Ser Ser Leu Thr Pro Glu Asp Thr Ala Val Tyr Tyr Cys
Ala Arg Trp Gly His Thr Thr Gly Thr Arg Phe Asp His Trp Gly Gln
Gly Val Met Val Thr Val Ser Ser
2D2 Light-Chain Variable Region (VL)
4D7 VL Nucleotide Sequence (SEQ ID NO: 10):
GATGTTCTGT TGACACAGAC TCCAGTTTCC CTGTCTGTCT CACTTGGAGA CCAAGCTTCT
ATATCTTGCA GGTCTAGTCA GAGCCTGGAA TATAGTGATG GATACACTTA TTTGGAATGG
TACGTACAGA AGCCAGGCCA GTCTCCACAG CTCCTCATCT ATGAAGTTTC CGACCGATTT
TCTGGGGTCC CAGACAGGTT CATTGGCAGT GGGTCAGGGA CAGATTTCAC CCTCAAGATC
AGCAGGGTAG AACCTGAGGA CTTGGGAGTT TATTACTGCT TCCAGGCTAC ACATGATCCT
CCGACGTTCG GTGGAGGCAC CAAGCTGGAA TTGAAA
2D2 VL Amino Acid Sequence (SEQ ID NO: 11):
Asp Val Leu Leu Thr Gln Thr Pro Val Ser Leu Ser Val thr Leu Gly
Asp Gln Ala Ser Ile Ser Cys Arg Ser Ser Gln Ser Leu Glu Tyr Ser
Asp Gly Tyr Thr Tyr Leu Glu Trp Tyr Val Gln Lys Pro Gly Gln Ser
Pro Gln Leu Leu Ile Tyr Glu Val Ser Asp Arg Phe Ser Gly Val Pro
Asp Arg Phe Ile Gly Ser Gly Ser Gly Thr Asp Phe Thr Leu Lys Ile
Ser Arg Val Glu Pro Glu Asp Leu Gly Val Tyr Tyr Cys Phe Gln Ala
Thr His Asp Pro Pro Thr Phe Gly Gly Gly Thr Lys Leu Glu Leu Lys

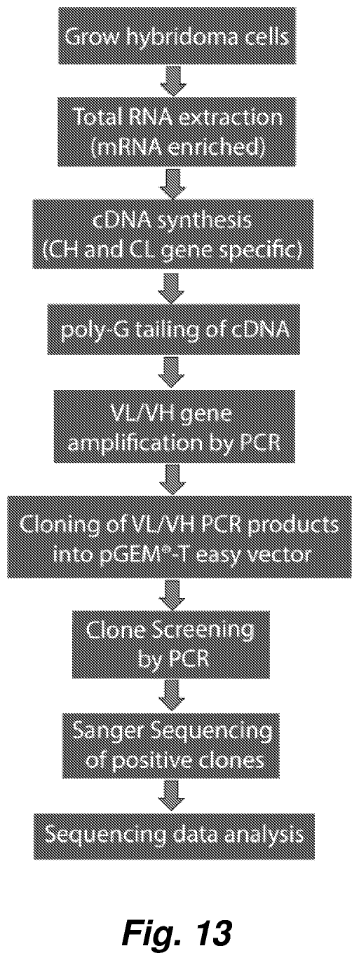

The results for 9H11 are shown in FIGS. 13 and 14.

9H11 Heavy-Chain Variable Region (VH)
9H11 VH Nucleotide Sequence
(SEQ ID NO: 12)
CAGGTCCAGT TGCAGCAGTC TGGAGCTGAG CTGACAAAGC CTGGCTCCTC AGTCAAGATT
TCCTGCAAGG CTTCTGGCTA CACCTTTACC ACCTACGATA TAAGCTGGAT AAAGCAGAGG
CCTGGACAGG CCCTTGAGTG GATTGGAGCA ATCTATCCAG GAGGTGAGAC AACAGGCTAC
AATGAGAAGT TCAAGGACAA GGCCACATTG ACTGTAGACA AATCTTCCAA CACGGCCTTC
ATGCAACTCA GCAGCCTGAC GCCTGAGGAC ACCGCGGTCT ATTACTGTGC AAGATGGGGT
CATACCACGG GTACCCGATT TGATCACTGG GGCCAAGGAG TCATGGTCAC AGTCTCCTCA
9H11 VH Amino Acid Sequence (SEQ ID NO: 13):
Gln Val Gln Leu Gln Gln Ser Gly Ala Glu Leu Thr Lys Pro Gly Ser
Ser Val Lys Ile Ser Cys Lys Ala Ser Gly Tyr Thr Phe Thr Thr Tyr
Asp Ile Ser Trp Ile Lys Gln Arg Pro Gly Gln Ala Leu Glu Trp Ile
Gly Ala Ile Tyr Pro Gly Gly Glu Thr Thr Gly Tyr Asn Glu Lys Phe
Lys Asp Lys Ala Thr Leu Thr Val Asp Lys Ser Ser Asn Thr Ala Phe
Met Gln Leu Ser Ser Leu Thr Pro Glu Asp Thr Ala Val Tyr Tyr Cys
Ala Arg Trp Gly His Thr Thr Gly Thr Arg Phe Asp His Trp Gly Gln
Gly Val Met Val Thr Val Ser Ser
9H11 Light-Chain Variable Region (VL)
9H11 VL Nucleotide Sequence (SEQ ID NO: 14):
GATATTGTGT TGACACAGAC TCCAGTTTCC CTGTCTGTCT CACTTGGAGA CCAAGCTTCT
ATATCTTGCA GGTCTAGTCA GAGCCTGGAA TATAGTGATG GATATACTTA TTTGGAATGG
TACGTACAGA AGCCAGGCCA GTCTCCACAG CTCCTCATCT GGGAAGTCTC CGACCGATTT
TCTGGGGTCC CAGATAGGTT CATTGGCAGT GGGTCAGGGA CAGACTTTAC CCTCAAGATC
AGCAGGGTAG AGCCTGAGGA CTTGGGAGTT TATTACTGCT TCCAGGCTAC ACATGATCCT
CCGACGTTCG GTGGAGGCAC CAAGCTGGAA TTGAAA
9H11 VL Amino Acid Sequence (SEQ ID NO: 15):
Asp Ile Val Leu Thr Gln Thr Pro Val Ser Leu Ser Val Ser Leu Gly
Asp Gln Ala Ser Ile Ser Cys Arg Ser Ser Gln Ser Leu Glu Tyr Ser
Asp Gly Tyr Thr Tyr Leu Glu Trp Tyr Val Gln Lys Pro Gly Gln Ser
Pro Gln Leu Leu Ile Trp Glu Val Ser Asp Arg Phe Ser Gly Val Pro
Asp Arg Phe Ile Gly Ser Gly Ser Gly Thr Asp Phe Thr Leu Lys Ile
Ser Arg Val Glu Pro Glu Asp Leu Gly Val Tyr Tyr Cys Phe Gln Ala
Thr His Asp Pro Pro Thr Phe Gly Gly Gly Thr Lys Leu Glu Leu Lys

The CDR1, CDR2 an CDR3 regions for each of the V H and V L variable regions for the antibodies 4D7, 2D2 and 9H11, and a consensus sequence for each of the CDRs is shown in FIGS. 15 and 16.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A method of preventing and/or treating a myeloproliferative disorder in a subject, the myeloproliferative disorder associated with the presence of a frameshift mutation in the calreticulin (CALR) gene of a cell involved in the myeloproliferative disorder, the method comprising exposing the subject, and/or cells involved in the myeloproliferative disorder, to an effective amount of an immunological agent that binds to an epitope in the CALR protein arising from the frameshift mutation in the CALR gene.

2. The method according to claim 1, wherein the frameshift mutation; (i) is a +1 frameshift mutation; or (ii) comprises a frameshift mutation in exon 9 of the CALR gene; or (iii) comprises a CALR^{del52} frameshift mutation or a CALR^{ins5} frameshift mutation.

3.-4. (canceled)

5. The method according to claim 1, wherein the disorder comprises primary myelofibrosis, ruxolitinib-resistant primary myelofibrosis, thrombocythemia, or secondary acute myeloid leukemia.

6. (canceled)

7. The method according to claim 1, wherein the immunological agent comprises an antibody and/or an antigen binding part thereof, or a monoclonal antibody.

8. (canceled)

9. The method according to claim 1, wherein the immunological agent binds to: (i) SEQ ID NO: 1; or (ii) an epitope in the N-terminus of SEQ ID NO: 1; or (iii) an epitope in the C-terminus of SEQ ID NO:1.

10.-11. (canceled)

12. The method according to claim 1, wherein the immunological agent inhibits the binding of dimers of the mutant CALR protein to thrombopoietin receptor.

13. The method according to claim 1, wherein the immunological agent comprises an antibody, or an antigen binding part thereof, comprising:
(i) an antibody heavy chain variable region comprising complementarity determining regions comprising CDR1 of SEQ ID NO: 18, a CDR2 of SEQ ID NO: 22 and a CDR3 of SEQ ID NO:26, and
an antibody light chain variable region comprising complementarity determining regions comprising CDR1 of SEQ ID NO:27, a CDR2 of SEQ ID NO:30 and a CDR3 of SEQ ID NO:31; or
(ii) an antibody heavy chain variable region comprising complementarity determining regions comprising a CDR1 of SEQ ID NO:16 or 17, a CDR2 of SEQ ID NO: 19, 20 or 21 and a CDR3 of SEQ ID NO:23, 24 or 25, and
an antibody light chain variable region comprising complementarity determining regions comprising a CDR1 of SEQ ID NO:27, a CDR2 of SEQ ID NO:28 or 29, and a CDR3 of SEQ ID NO: 31; or
(iii) an antibody heavy chain variable region comprising complementarity determining regions comprising SEQ ID NO: 5 and an antibody light chain variable region comprising complementarity determining regions comprising SEQ ID NO: 7 (4D7); or
(iv) an antibody heavy chain variable region comprising complementarity determining regions comprising SEQ ID NO: 9 and an antibody light chain variable region comprising the complementarity determining regions comprising SEQ ID NO: 11 (2D2); or
(v) an antibody heavy chain variable region comprising complementarity determining regions comprising SEQ ID NO: 13 and an antibody light chain variable region comprising complementarity determining regions comprising SEQ ID NO: 15 (9H11).

14.-17. (canceled)

18. The method according to claim 1, wherein the immunological agent is coupled to a therapeutic agent.

19. (canceled)

20. An immunological agent, or an antibody or an antigen binding part thereof, that binds to an epitope in a mutant CALR protein, wherein the mutant CALR protein arises from a frameshift mutation in the CALR gene.

21. An immunological agent, or an antibody or an antigen binding part thereof, according to claim 20, that inhibits the binding of dimers of the mutant CALR protein to thrombopoietin receptor.

22. The immunological agent, or an antibody or an antigen binding part thereof, according to claim 20, wherein the frameshift mutation: (i) is a +1 frameshift mutation; or (ii) comprises a frameshift mutation in exon 9 of the CALR gene; or (iii) comprises a CALR^{del52} or a CALR^{ins5} frameshift mutation.

23.-25. (canceled)

26. The immunological agent, or an antibody or an antigen binding part thereof, according to claim 20, wherein the antibody comprises a monoclonal antibody.

27. The immunological agent, or an antibody or an antigen binding part thereof, according to claim 20, that inhibits: (i) JAK-STAT signalling in a megakaryocyte cell and/or a progenitor cell thereof; or (ii) thrombopoietin-independent proliferation and/or differentiation of megakaryocytes and/or progenitor cells.

28. (canceled)

29. The immunological agent, or an antibody or an antigen binding part thereof, according to claim 20, wherein the antibody, or an antigen binding part thereof, comprises:
(i) an antibody heavy chain variable region comprising complementarity determining regions comprising a CDR1 of SEQ ID NO: 18, a CDR2 of SEQ ID NO: 22 and a CDR3 of SEQ ID NO:26, and
an antibody light chain variable region comprising complementarity determining regions comprising a CDR1 of SEQ ID NO:27, a CDR2 of SEQ ID NO:30 and a CDR3 of SEQ ID NO:31; or
(ii) an antibody heavy chain variable region comprising complementarity determining regions comprising a CDR1 of SEQ ID NO:16 or 17, a CDR2 of SEQ ID NO: 19, 20 or 21 and a CDR3 of SEQ ID NO:23, 24 or 25, and
an antibody light chain variable region comprising complementarity determining regions comprising a CDR1 of SEQ ID NO:27, a CDR2 of SEQ ID NO:28 or 29, and a CDR3 of SEQ ID NO: 31; or
(iii) an antibody heavy chain variable region comprising complementarity determining regions comprising SEQ ID NO: 5 and an antibody light chain variable region comprising complementarity determining regions comprising SEQ ID NO: 7 (4D7); or
(iv) an antibody heavy chain variable region comprising complementarity determining regions comprising SEQ ID NO: 9 and an antibody light chain variable region comprising the complementarity determining regions comprising SEQ ID NO: 11 (2D2); or
(v) an antibody heavy chain variable region comprising complementarity determining regions comprising SEQ ID NO: 13 and an antibody light chain variable region comprising complementarity determining regions comprising SEQ ID NO: 15 (9H11).

30.-46. (canceled)

47. The immunological agent, or an antibody or an antigen binding part thereof, according to claim 20, wherein the antibody or the antigen binding part thereof are humanised.

48. (canceled)

49. A chimeric antigen receptor comprising an extracellular domain having a binding domain that binds to an epitope in a CALR protein arising from a frameshift mutation in the CALR gene.

50. The chimeric antigen receptor according to claim 49, wherein the frameshift mutation; (i) is a +1 frameshift mutation; or (ii) comprises a frameshift mutation in exon 9 of the CALR gene; or (iii) comprises a CALR^{del52} or a CALR^{ins5} frameshift mutation.

51.-53. (canceled)

54. The chimeric antigen receptor according to claim 49, wherein the binding domain comprises:
(i) complementarity determining regions comprising a CDR1 of SEQ ID NO: 18, a CDR2 of SEQ ID NO:22 and a CDR3 of SEQ ID NO:26, or a functional variant having at least 90% sequence identity to any one or more of the aforementioned complementarity determining regions; or
(ii) complementarity determining regions comprising a CDR1 of SEQ ID NO:27, a CDR2 of SEQ ID NO: 30 and a CDR3 of SEQ ID NO:31, or a functional variant having at least 90% sequence identity to any one or more of the aforementioned complementarity determining regions; or
(iii) complementarity determining regions comprising a CDR1 of SEQ ID NO:16 or 17, a CDR2 of SEQ ID NO: 19, 20 or 21, and a CDR3 of SEQ ID NO:23, 24 or 25; or
(iv) complementarity determining regions comprising a CDR1 of SEQ ID NO:27, a CDR2 of SEQ ID NO:28 or 29, and a CDR3 of SEQ ID NO:31.

55.-56. (canceled)

57. A method of preventing and/or treating a myeloproliferative disorder in a subject, the myeloproliferative disorder associated with the presence of a frameshift mutation in the CALR gene of a cell involved in the myeloproliferative disorder, the method comprising exposing the subject, and/or cells involved in the myeloproliferative disorder, to T cells expressing the chimeric antigen receptor according to claim 49.

58.-62. (canceled)

63. A method of preventing and/or treating a myeloproliferative disorder in a subject, the disorder associated with the presence of a frameshift mutation in the CALR gene of a cell involved in the myeloproliferative disorder, the method comprising exposing the subject, and/or cells involved in the myeloproliferative disorder, to an effective amount of an immunological agent, or an antibody or an antigen binding part thereof, according to claim 20.


SEQUENCE LISTING (33 sequences)

SEQ ID NO: 1 - protein, 44 aa, Rattus rattus
RRMMRTKMRMRRMRRTRRKMRRKMSPARPRTSCREACLQGWTEA

SEQ ID NO: 2 - protein, 30 aa, Rattus rattus
MMRTKMRMRRMRRTRRKMRRKMSPARPRTS

SEQ ID NO: 3 - protein, 11 aa, Rattus rattus
RKMSPARPRTS

SEQ ID NO: 4 - DNA, 360 bp, Rattus rattus
  source 1..360: mol_type = other DNA
caggtccaactgcagcagtctggtgctgagctggcaaagcctggctcttcagtgaaaatttcctgcaaggcttctggctacacctttaccaactacgatatgagctggataaagcagaggcctggacaggcccttgagtggattggagccattaatccaggaaatggagttacagactacaatgagaagttcaggggcaaggccacattgactgtagacaaatcctccagcacagccttcatgcaactcagcagcctgacacctgaggacactgcggtctatcactgtgcaagagaatatagggacaactacggctactttgattactggggccaaggagtcatggtcacagtctcctca

SEQ ID NO: 5 - protein, 120 aa, Rattus rattus
QVQLQQSGAELAKPGSSVKISCKASGYTFTNYDMSWIKQRPGQALEWIGAINPGNGVTDYNEKFRGKATLTVDKSSSTAFMQLSSLTPEDTAVYHCAREYRDNYGYFDYWGQGVMVTVSS

SEQ ID NO: 6 - DNA, 336 bp, Rattus rattus
  source 1..336: mol_type = other DNA
gatattgtgttgacacaaactccagtttccctgtctgtcacacttggagatcaagcttctatatcttgcaggtctagtcagagcctggaatatagtgatggatacacttatttggattggttcctacagaagccaggccagtctccacagctcctcatctatgaagtttccagccgattttctggggtcccagacaggttcattggccgtgggtcagggacagatttcaccctcaagatcagcagagtagagcctgaggacttgggagtttattactgcttccaagctacacatgatccattcacgttcggctcagggacgaagttggaaataaaa

SEQ ID NO: 7 - protein, 112 aa, Rattus rattus
DIVLTQTPVSLSVTLGDQASISCRSSQSLEYSDGYTYLDWFLQKPGQSPQLLIYEVSSRFSGVPDRFIGRGSGTDFTLKISRVEPEDLGVYYCFQATHDPFTFGSGTKLEIK

SEQ ID NO: 8 - DNA, 360 bp, Rattus rattus
  source 1..360: mol_type = other DNA
caggtccagttgcagcagtctggagctgagctgacaaagcctggctcttcagtgaagatttcctgcaagacttctggctacacctttaccacctacgatataagctggataaaacagaggcctggacaggcccttgagtggattggagcaatctatccaggaggtgtaactacaggctacaatgagaagttcaagggcaaggccacattgactgtagacaaatcttccaacacggccttcatgcaactcagcagcctgacacctgaggacactgcggtctattactgtgcaagatggggtcataccacgggtacccgatttgatcactggggccaaggagtcatggtcacagtctcctca

SEQ ID NO: 9 - protein, 120 aa, Rattus rattus
QVQLQQSGAELTKPGSSVKISCKTSGYTFTTYDISWIKQRPGQALEWIGAIYPGGVTTGYNEKFKGKATLTVDKSSNTAFMQLSSLTPEDTAVYYCARWGHTTGTRFDHWGQGVMVTVSS

SEQ ID NO: 10 - DNA, 336 bp, Rattus rattus
  source 1..336: mol_type = other DNA
gatgttctgttgacacagactccagtttccctgtctgtctcacttggagaccaagcttctatatcttgcaggtctagtcagagcctggaatatagtgatggatacacttatttggaatggtacgtacagaagccaggccagtctccacagctcctcatctatgaagtttccgaccgattttctggggtcccagacaggttcattggcagtgggtcagggacagatttcaccctcaagatcagcagggtagaacctgaggacttgggagtttattactgcttccaggctacacatgatcctccgacgttcggtggaggcaccaagctggaattgaaa

SEQ ID NO: 11 - protein, 112 aa, Rattus rattus
DVLLTQTPVSLSVSLGDQASISCRSSQSLEYSDGYTYLEWYVQKPGQSPQLLIYEVSDRFSGVPDRFIGSGSGTDFTLKISRVEPEDLGVYYCFQATHDPPTFGGGTKLELK

SEQ ID NO: 12 - DNA, 360 bp, Rattus rattus
  source 1..360: mol_type = other DNA
caggtccagttgcagcagtctggagctgagctgacaaagcctggctcctcagtcaagatttcctgcaaggcttctggctacacctttaccacctacgatataagctggataaagcagaggcctggacaggcccttgagtggattggagcaatctatccaggaggtgagacaacaggctacaatgagaagttcaaggacaaggccacattgactgtagacaaatcttccaacacggccttcatgcaactcagcagcctgacgcctgaggacaccgcggtctattactgtgcaagatggggtcataccacgggtacccgatttgatcactggggccaaggagtcatggtcacagtctcctca

SEQ ID NO: 13 - protein, 120 aa, Rattus rattus
QVQLQQSGAELTKPGSSVKISCKASGYTFTTYDISWIKQRPGQALEWIGAIYPGGETTGYNEKFKDKATLTVDKSSNTAFMQLSSLTPEDTAVYYCARWGHTTGTRFDHWGQGVMVTVSS

SEQ ID NO: 14 - DNA, 336 bp, Rattus rattus
  source 1..336: mol_type = other DNA
gatattgtgttgacacagactccagtttccctgtctgtctcacttggagaccaagcttctatatcttgcaggtctagtcagagcctggaatatagtgatggatatacttatttggaatggtacgtacagaagccaggccagtctccacagctcctcatctgggaagtctccgaccgattttctggggtcccagataggttcattggcagtgggtcagggacagactttaccctcaagatcagcagggtagagcctgaggacttgggagtttattactgcttccaggctacacatgatcctccgacgttcggtggaggcaccaagctggaattgaaa

SEQ ID NO: 15 - protein, 112 aa, Rattus rattus
DIVLTQTPVSLSVSLGDQASISCRSSQSLEYSDGYTYLEWYVQKPGQSPQLLIWEVSDRFSGVPDRFIGSGSGTDFTLKISRVEPEDLGVYYCFQATHDPPTFGGGTKLELK

SEQ ID NO: 16 - protein, 8 aa, Rattus rattus
GYTFTNYD

SEQ ID NO: 17 - protein, 8 aa, Rattus rattus
GYTFTTYD

SEQ ID NO: 18 - protein, 8 aa, Rattus rattus
  VARIANT 6: note = X = T or N
GYTTTXYD

SEQ ID NO: 19 - protein, 8 aa, Rattus rattus
INPGNGVT

SEQ ID NO: 20 - protein, 8 aa, Rattus rattus
IYPGGVTT

SEQ ID NO: 21 - protein, 8 aa, Rattus rattus
IYPGGETT

SEQ ID NO: 22 - protein, 8 aa, Rattus rattus
  VARIANT 2: note = X = Y or N
  VARIANT 5: note = X = G or N
  VARIANT 6: note = X = G, V or E
  VARIANT 7: note = X = T or V
IXPGXXXT

SEQ ID NO: 23 - protein, 13 aa, Rattus rattus
AREYRDNYGYFDY

SEQ ID NO: 24 - protein, 13 aa, Rattus rattus
ARWGHTTGTRFDH

SEQ ID NO: 25 - protein, 13 aa, Rattus rattus
ARWGHTTGTRFDK

SEQ ID NO: 26 - protein, 13 aa, Rattus rattus
  VARIANT 3: note = X = W or E
  VARIANT 4: note = X = G or Y
  VARIANT 5: note = X = H or R
  VARIANT 6: note = X = T or D
  VARIANT 7: note = X = T or N
  VARIANT 8: note = X = G or Y
  VARIANT 9: note = X = T or G
  VARIANT 10: note = X = R or Y
  VARIANT 13: note = X = H or Y
ARXXXXXXXXFDX

SEQ ID NO: 27 - protein, 11 aa, Rattus rattus
QSLEYSDGYTY

SEQ ID NO: 28 - protein, 8 aa, Rattus rattus
EVSSRFSG

SEQ ID NO: 29 - protein, 8 aa, Rattus rattus
EVSDRFSG

SEQ ID NO: 30 - protein, 8 aa, Rattus rattus
  VARIANT 4: note = X = D or S
EVSXRFSG

SEQ ID NO: 31 - protein, 9 aa, Rattus rattus
FQATHDPFT

SEQ ID NO: 32 - DNA, 34 bp, synthetic construct
  source 1..34: mol_type = other DNA
tatagaattcgccaccatgctgctatccgtgccg

SEQ ID NO: 33 - DNA, 28 bp, synthetic construct
  source 1..28: mol_type = other DNA
tatagaattcaggcctcagtccagccct